(19) United States
(12) Patent Application Publication
(10) Pub. No.: US 2024/0293351 A1
(43) Pub. Date: Sep. 5, 2024

(54) METHODS OF ADMINISTERING SYNTHETIC TRITERPENOIDS

(71) Applicant: REATA PHARMACEUTICALS HOLDINGS, LLC, Plano, TX (US)

(72) Inventors: Scott Aaron REISMAN, Fort Worth, TX (US); Sarabjit GAHIR, Plano, TX (US)

(73) Assignee: REATA PHARMACEUTICALS HOLDINGS, LLC, Plano, TX (US)

(21) Appl. No.: 18/256,545

(22) PCT Filed: Dec. 10, 2021
(86) PCT No.: PCT/US2021/072851

§ 371 Date: Jun. 8, 2023

Related U.S. Application Data
(60) Provisional application No. 63/124,677, filed on Dec. 11, 2020.

(51) Int. Cl. A61K 31/277; A61K 9/00

(52) U.S. Cl. CPC A61K 31/277; A61K 9/0053

(57) ABSTRACT

Provided are methods of administering synthetic tri terpenoids, such as bardoxolone methyl or omaveloxolone, to a patient in need thereof while avoiding adverse drug interactions with cytochrome P450 3A4 (CYP3A4) modulators. Such treatment methods comprise avoiding, contraindicating, or discontinuing concomitant use or co-administration of a cytochrome P450 3A4 modulator.

### REFERENCE TO RELATED APPLICATIONS

The present application claims the priority benefit of U.S. provisional application No. 63/124,677, filed Dec. 11, 2020, the entire contents of which is incorporated herein by reference.

### BACKGROUND

### 1. Field

The present invention relates generally to the field of medicine. More particularly, it concerns methods of administering synthetic triterpenoids while avoiding drug-drug interactions with CYP3A4 modulators.

### 2. Description of Related Art

Bardoxolone methyl (RTA 402), omaveloxolone (RTA 408), and related triterpenoid analogs are among the most potent known activators of nuclear factor erythroid-derived 2-related factor 2 (Nrf2) and are also inhibitors of nuclear factor kappa-light-chain enhancer of activated B-cells (NF-κB), thus inducing an anti-inflammatory and anti-oxidative phenotype. Nrf2 signaling promotes anti-oxidative mechanisms (Muthusamy et al., 2012) and Nrf2 activation can increase mitochondrial respiration (Holmostrom et al., 2013; Ludtmann et al., 2014). Because of this mechanism of action, bardoxolone methyl, omaveloxolone, and their analogs are hypothesized to have potential therapeutic relevance in a variety of disease settings involving oxidative stress and inflammation.

Synthetic triterpenoids are extensively metabolized by hepatic enzymes to multiple oxidative metabolites. Taking bardoxolone methyl as an example, three metabolites have been identified in human plasma, none of which contribute to the pharmacological activity of bardoxolone methyl. The cytochrome P450 enzyme system (CYP450) is responsible for the biotransformation of drugs from active substances to inactive metabolites that can be excreted from the body. In addition, the metabolism of certain drugs by CYP450 can alter their PK profile. One important subtype of CYP450 is CYP3A4. There is a significant, unmet need for understanding whether modulators of CYP3A4 affect plasma concentrations of synthetic triterpenoids. Since such drug-drug interactions may increase or decrease the effects of one or both drugs, it is important to understand these effects in order to minimize side effects and maximize pharmacological effects. The present disclosure fulfills these and other needs, as evident in reference to the following disclosure.

### SUMMARY

In one aspect, provided herein are methods of treating a patient in need thereof with a compound of the formula and/or selecting a patient in need thereof for treatment with a compound of the formula:

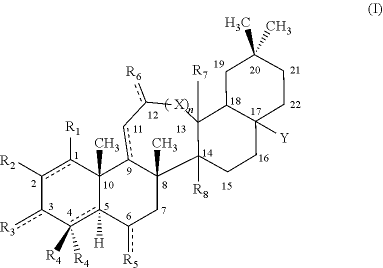

wherein:
the bond between atoms 1 and 2 is a single bond, a double bond, or an epoxidized double bond;
the bond between atoms 4 and 5 is a single bond or a double bond;
the bond between atoms 9 and 11 is a single bond or a double bond;
n is 0 or 1;
X is —O— or —NH—;
R₁ is hydrogen, hydroxy, alkyl_{(C≤8)}, or substituted alkyl_{(C≤8)};
R₂ is —CN, halo, hydrogen, hydroxy, or —CF₃; or
   heteroaryl_{(C≤8)} or substituted heteroaryl_{(C≤8)}; or
   —C(O)R_{b}, wherein R_{b} is —OH, alkoxy_{(C1-4)}, —NH₂, alkylamino_{(C1-4)}, —NH—S(O)₂-alkyl_{(C1-4)}, or —NHOH;
R₃ hydroxy or oxo; or
   alkoxy_{(C≤8)}, substituted alkoxy_{(C≤8)}, acyloxy_{(C≤8)}, or substituted acyloxy_{(C≤8)};
R₄ and R₄′ are each independently hydrogen, amino, cyano, or halo; or
   alkyl_{(C≤8)}, cycloalkyl_{(C≤8)}, heteroaryl_{(C≤8)} acyl_{(C≤8)}, amido_{(C≤8)}, alkylamino_{(C≤8)}, dialkylamino_{(C≤8)}, or a substituted version of any of these groups; or
   —OR_{c}, wherein:
      R_{c} is hydrogen or alkyl_{(C≤8)}, alkenyl_{(C≤8)}, alkynyl_{(C≤8)}, aryl_{(C≤12)}, aralkyl_{(C≤12)}, heteroaryl_{(C≤12)}, heterocycloalkyl_{(C≤8)}, acyl_{(C≤8)}, or a substituted version of any of these groups;
   (CH₂)ₚ—(OCH₂)_{q}—R₉, wherein:
      R₉ is hydroxy or acyl_{(C≤8)}, alkoxy_{(C≤8)}, acyloxy_{(C≤8)}, alkylsilyloxy_{(C≤8)}, or a substituted version of any of these groups;
      p is 0, 1, 2, 3, or 4; and
      q is 0, 1, 2, or 3;
   (CH₂)ₛNR₁₀(R₁₀′), wherein:
      s is 0, 1, 2, 3, or 4;
      R₁₀ is hydrogen, alkyl_{(C≤8)}, alkoxy_{(C≤8)}, substituted alkoxy_{(C≤8)}, acyl_{(C≤8)} substituted acyl_{(C≤8)}, —C(O)-alkoxy_{(C≤8)}, substituted —C(O)-alkoxy_{(C≤8)}, acyloxy_{(C≤8)}, substituted acyloxy_{(C≤8)}, alkylsilyloxy_{(C≤8)} or substituted alkylsilyloxy_{(C≤8)}; and
      R₁₀′ is hydrogen, alkyl_{(C≤8)}, or substituted alkyl_{(C≤8)}; or
   —(CH₂)ₜ—C(O)—R₁₁, wherein:
      R₁₁ is amino, hydroxy, or mercapto; or alkoxy_{(C≤8)} alkylthio_{(C≤8)}, alkylamino_{(C≤8)}, dialkyl-amino_{(C≤8)}, or a substituted version of any of these groups; and
      t is 0, 1, 2, 3, or 4; or
R₄ and R₄′ are taken together and are alkylidene_{(C≤8)};
R₅ is hydrogen, hydroxy, or oxo;
R₆ is halo, hydrogen, hydroxy, oxo, or ═N(OH); or alkoxy_{(C≤8)}, substituted alkoxy_{(C≤8)}, acyloxy_{(C≤8)}, or substituted acyloxy_{(C≤8)};
R₇ and R₈ are each independently hydrogen, hydroxy, or methyl or as defined below when either of these groups is taken together with group R_{c};
Y is:
   amino, halo, cyano, hydrogen, hydroxy, mercapto, —CF₃, or —NCO;
   alkyl_{(C≤8)}, cycloalkyl_{(C≤8)}, alkenyl_{(C≤8)}, alkynyl_{(C≤8)}, aryl_{(C≤12)}, aralkyl_{(C≤12)}, heteroaryl_{(C≤8)}, heterocycloalkyl_{(C≤12)}, alkoxy_{(C≤8)}, cycloalkoxy_{(C≤8)}, aryloxy_{(C≤12)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, heteroaralkylamino_{(C≤8)}, alkylthio_{(C≤8)}, acylthio_{(C≤8)}, alkylsulfonylamino_{(C≤8)}, arylsulfonylamino_{(C≤12)}, heteroarylsulfonylamino_{(C≤12)}, cycloalkylsulfonylaminocs), -heteroarenediyl_{(C≤8)}-cycloalkyl_{(C≤8)}, or substituted versions of any of these groups;
   alkanediyl_{(C≤8)}—R_{d}, -alkenediyl_{(C≤8)}—R_{d}, or a substituted version of any of these groups, wherein R_{d} is:
      heteroaryl_{(C≤8)}, heterocycloalkyl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, heteroarylamino_{(C≤8)}, heteroaralkylamino_{(C≤8)}, alkylsulfonylamino_{(C≤8)}, cyclo-alkylsulfonylamino_{(C≤8)}, amido_{(C≤8)}, —NHC(O)-cycloalkyl_{(C≤8)}, —OC(O)NH-alkyl_{(C≤8)}, or a substituted version of any of these groups;
   (CH₂)ᵤC(O)Rₑ, wherein u is 0-6 and Rₑ is:
      hydrogen, hydroxy, halo, amino, azido, —NHOH, or mercapto; or
      alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, heterocycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino(ccs), cycloalkylamino_{(C≤8)}, dialkylamino_{(C≤8)}, arylamino_{(C≤8)}, heteroarylamino_{(C≤8)}, alkylsulfonylamino(ccs), cycloalkylsulfonylamino_{(C≤8)}, amido_{(C≤8)}, —NH-alkoxy_{(C≤8)}, —NH-heterocycloalkyl_{(C≤8)}, —NH-amido_{(C≤8)}, —O-alkanediyl_{(C≤8)}-heterocycloalkyl_{(C≤8)} or a substituted version of any of these groups; and
   —NHC(O)R_{f}, wherein R_{f} is:
      hydrogen, hydroxy, or amino; or
      alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, alkenyloxy_{(C≤8)}, cycloalkoxy_{(C≤8)}, heterocycloalkoxy_{(C≤8)}, aryloxy_{(C≤8)}, aralkoxy_{(C≤8)}, heteroaryloxy_{(C≤8)}, acyloxy_{(C≤8)}, alkylamino(ccs), cycloalkylamino_{(C≤8)}, dialkylamino_{(C≤8)}, arylamino_{(C≤8)}, or a substituted version of any of these groups; or
Y is taken together with either R₇ or R₈ and is —(CH₂)ᵥC(O)R_{g}—, wherein
   v is 0-6; and
   R_{g} is —O— or —NR₁₂—; wherein:
      R₇ is hydrogen, alkyl_{(C≤8)} substituted alkyl_{(C≤8)}, acyl_{(C≤8)}, or substituted acyl_{(C≤8)}; or
         or a pharmaceutically acceptable salt thereof. Such methods are described in the sections below, including for example the claims section, which is incorporated herein by reference.

In some embodiments, the methods comprising (i) determining or having determined whether a patient is currently being administered a CYP3A4 modulator; and (ii) selecting or having selected the patient for treatment with the compound if the patient is not currently being administered a CYP3A4 modulator. In some embodiments, the methods further comprise (iii) administering or having administered a therapeutically effective amount of the compound to the selected patient.

In some embodiments, the methods comprise administering a therapeutically effective amount of the compound to the patient, wherein the patient has discontinued concomitant use of a CYP3A4 modulator. In some embodiments, the methods comprise administering a therapeutically effective amount of the compound to the patient, wherein the patient has not been prescribed a CYP3A4 modulator. In some embodiments, the methods comprise administering a therapeutically effective amount of the compound to the patient, wherein the patient is not currently taking a CYP3A4 modulator. In some embodiments, the methods comprise administering a therapeutically effective amount of the compound to the patient, wherein the patient has not taken a CYP3A4 modulator within one week of starting administration of the compound.

In some embodiments, the methods comprise administering a therapeutically effective amount of the compound to the patient and avoiding, contraindicating, or discontinuing concomitant use or co-administration of a cytochrome P450 3A4 (CYP3A4) modulator to the patient. The administration of a CYP3A4 modulator may be avoided during administration of the compound. Administration of the CYP3A4 modulator may be discontinued prior to starting administration of the compound.

In some embodiment, when the patient is also in need of therapy with a CYP3A4 modulator, the methods comprise administering to the patient a therapeutically effective amount of the compound while avoiding co-administration of a CYP3A4 modulator to the patient, and any one or more of the following:
(a) advising the patient that a CYP3A4 modulator should be avoided or discontinued,
(b) advising the patient that co-administration of the compound with drugs that are moderate to strong modulators of CYP3A4 can alter the therapeutic effect or adverse reaction profile of the compound,
(c) advising the patient that co-administration of the compound with a CYP3A4 modulator can alter the therapeutic effect or adverse reaction profile of the compound,
(d) advising the patient that use of the compound in patients being treated with a CYP3A4 modulator is contraindicated, or
(e) advising the patient that co-administration of the compound and a CYP3A4 modulator resulted in a more than 5-fold increase in exposure to the compound.
   In some embodiments, the methods may further comprise discontinuing administration of a CYP3A4 modulator. In some embodiments, the methods may comprise (a) first discontinuing administration of a CYP3A4 modulator to the patient and (b) second administering a therapeutically effective amount of the compound to the patient.

In one embodiment, provided herein is a compound of formula (I) for use in the treatment of a patient in need of thereof, wherein the treatment comprises avoiding, contraindicating, or discontinuing concomitant use or co-administration of a cytochrome P450 3A4 modulator.

In one embodiment, provided herein is use of a compound of formula (I) in the preparation of a medicament for treatment of a patient in need of thereof, wherein the treatment comprises avoiding, contraindicating, or discontinuing concomitant use or co-administration of a cytochrome P450 3A4 modulator.

In some embodiments, the compound is administered locally. In some embodiments, the compound is administered systemically. In some embodiments, the compound is administered orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, orally, parenterally, rectally, subconjunctivally, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof. For example, in some variations, the compound is administered intravenously, intra-arterially or orally. For example, in some variations, the compound is administered orally.

In some embodiments, the patient is a mammal such as primate. In some variations, the primate is a human. In other variations, the patient is a cow, horse, dog, cat, pig, mouse, rat or guinea pig.

Any embodiment discussed herein with respect to one aspect of the invention applies to other aspects of the invention as well, unless specifically noted.

Further aspects and embodiments of this invention are elaborated in greater detail, for example, in the claims section, which is incorporated herein by reference.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Plots of Mean (±Standard Deviation) Plasma Bardoxolone Methyl Concentrations by Treatment on Linear Scale and Semi-Logarithmic Scale—Pharmacokinetic Concentration Population. Treatment A (Period 1) was a single dose of bardoxolone methyl. Treatment B (Period 2) was a single dose of bardoxolone methyl and daily doses of itraconazole. The lower limit of quantitation for bardoxolone methyl was 0.0500 ng/mL. If the actual sampling time (measured from dosing) was outside of the collection window for nominal time points, the corresponding concentration was excluded from concentration versus time descriptive summaries and plots but was still used in the calculation of pharmacokinetic parameters. SD=standard deviation.

FIG. 2. Study schematic. CRU=Clinical Research Unit; QD=once daily.

FIG. 3. Arithmetic Mean (+SD) Plasma Concentrations of Omaveloxolone.

FIGS. 4A-C. X-ray Powder Diffraction (XRPD) Spectra of Forms A and B of RTA 402. FIG. 4A shows unmicronized Form A; FIG. 4B shows micronized Form A; FIG. 4C shows Form B.

FIG. 5. PXRD (1.5-55.5° 2θ) pattern of sample PP415-P1, which corresponds to the amorphous form of RTA 408 (Class 1).

FIG. 6. DSC thermogram of the sample PP415-P1, which corresponds to the amorphous form of RTA 408 (Class 1).

FIG. 7. PXRD patterns (2-32° 2θ) of class 2 (sample PP415-P19: top), class 3 (sample PP415-P6: 2^{nd} from top), class 4 (sample PP415-P13: 2^{nd} from bottom), and class 5 (sample PP415-P14: bottom) RTA 408 are distinctly different. The patterns have been scaled and offset in the y-direction for the purpose of comparison.

FIG. 8. DSC thermogram of the desolvated acetonitrile solvate form (Class 4) of RTA 408 (sample PP415-P37).

FIG. 9. PXRD patterns (2-30° 2θ) of RTA 408 Form A.

FIG. 10. DSC thermogram (25-280° C.) of RTA 408 Form A.

FIG. 11. PXRD patterns (2-30° 2θ) of RTA 408 Form B.

FIG. 12. DSC thermogram (25-280° C.) of RTA 408 Form B.

### DETAILED DESCRIPTION

Provided herein are methods of avoiding adverse drug interactions between strong modulators of CYP3A4 and synthetic triterpenoids, such as, for example, bardoxolone and omaveloxolone.

### I. Synthetic Triterpenoid Compounds

Triterpenoids, biosynthesized in plants by the cyclization of squalene, are used for medicinal purposes in many Asian countries; and some, such as ursolic and oleanolic acid, are known to be anti-inflammatory and anti-carcinogenic (Huang et al., 1994; Nishino et al., 1988). However, the biological activity of these naturally occurring molecules is relatively weak, and therefore the synthesis of new analogs to enhance their potency was undertaken (Honda et al., 1997; Honda et al., 1998). An ongoing effort for the improvement of anti-inflammatory and antiproliferative activity of oleanolic and ursolic acid analogs led to the discovery of 2-cyano-3,12-dioxooleane-1,9(11)-dien-28-oic acid (CDDO) and related compounds (Honda et al., 1997, 1998, 1999, 2000a, 2000b, 2002; Suh et al., 1998; 1999; 2003; Place et al., 2003; Liby et al., 2005). Several potent derivatives of oleanolic acid were identified, including methyl-2-cyano-3,12-dioxooleana-1,9-dien-28-oic acid (CDDO-Me; RTA 402; BARD; bardoxolone methyl). These compounds activate the Keap1/Nrf2/ARE signaling pathway resulting in the production of several anti-inflammatory and antioxidant proteins, such as heme oxygenase-1 (HO-1).

Accordingly, in pathologies involving oxidative stress alone or oxidative stress exacerbated by inflammation, treatment may comprise administering to a subject or patient a therapeutically effective amount of a compound, such as those described above or throughout this specification. Treatment may be administered preventively in advance of a predictable state of oxidative stress (e.g., organ transplantation or the administration of therapy to a cancer patient), or it may be administered therapeutically in settings involving established oxidative stress and inflammation.

Non-limiting examples of synthetic triterpenoids that may be used in accordance with the methods of this invention include those disclosed in PCT Publn. Nos. WO 1999/065478; WO 2004/064723; WO 2008/136838; WO 2009/129545; WO 2009/129546; WO 2009/129548; WO 2009/146216; WO 2012/125488; WO 2013/163344; WO 2014/040056; WO 2014/040060; WO 2014/040073; WO 2015/027206; WO 2017/053868; WO 2018/089539; Int'l. Appln. No. PCT/US2020/042788; and U.S. Prov. Appln. No. 63/198,310, each of which is incorporated by reference herein in its entirety. Additional non-limiting examples of synthetic triterpenoids that may be used in accordance with the methods of this invention are shown here:

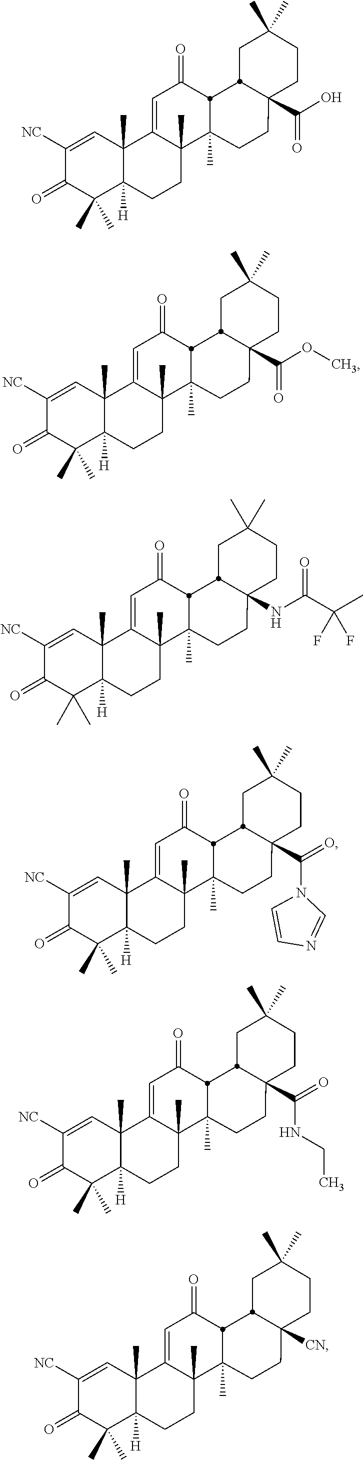

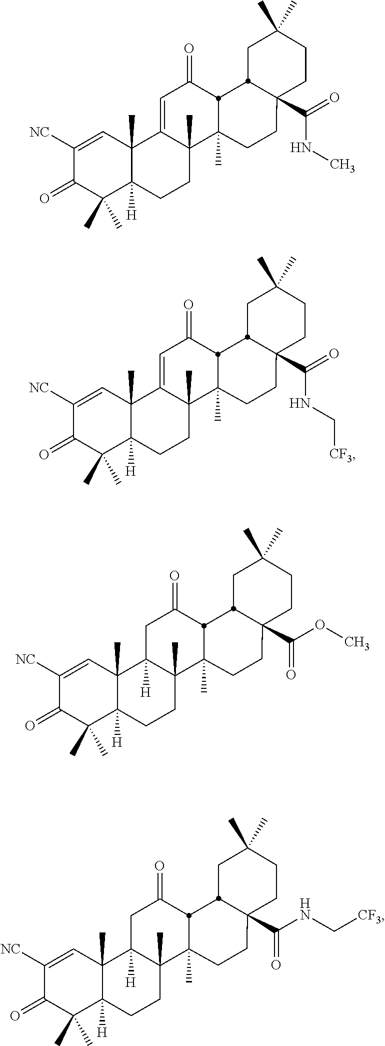

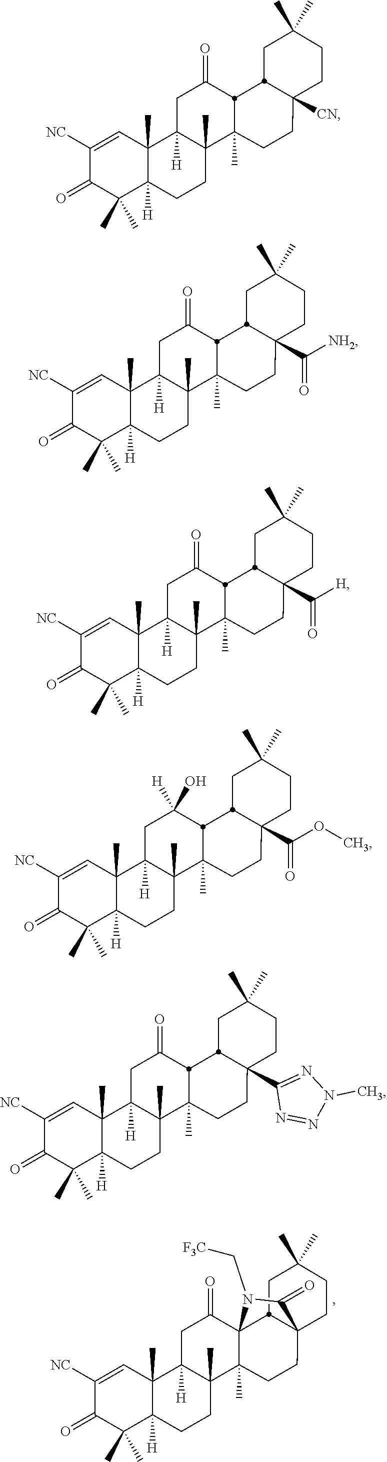

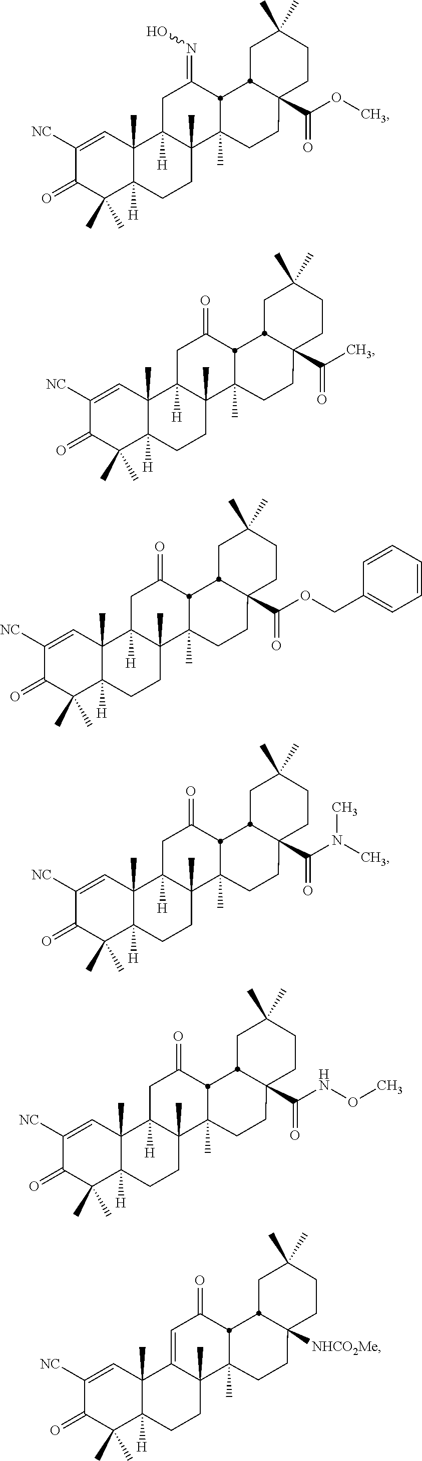

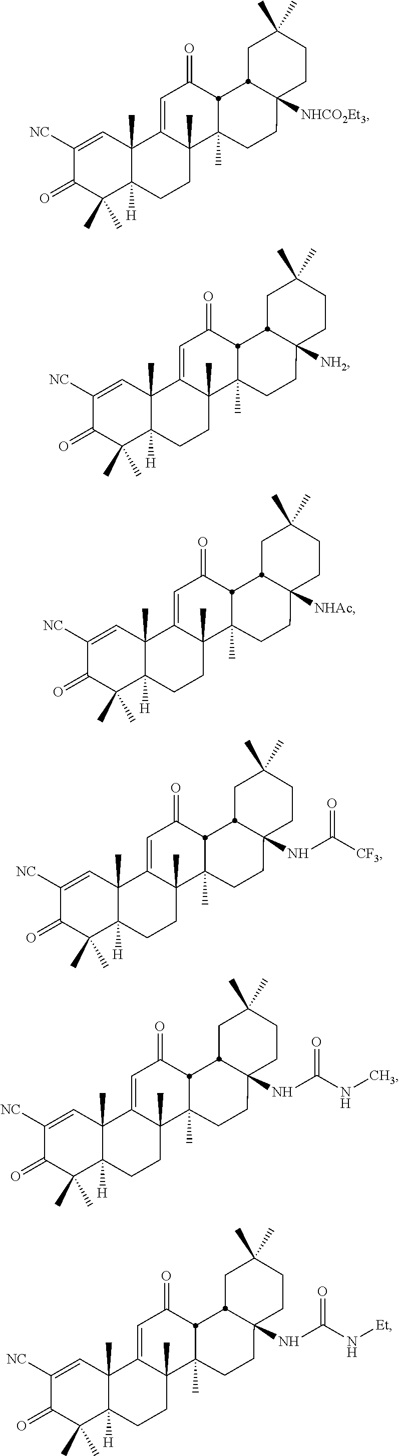

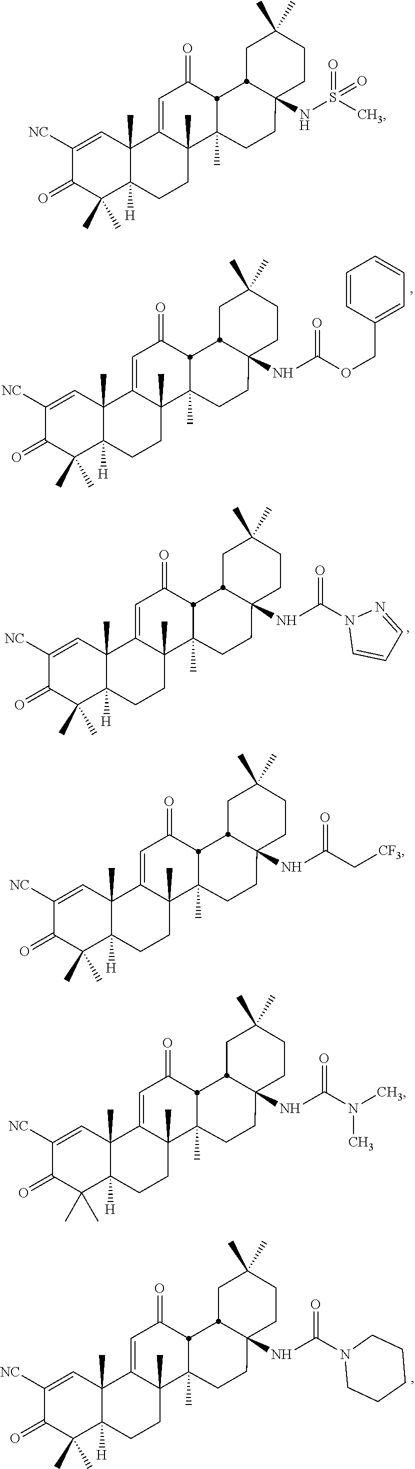

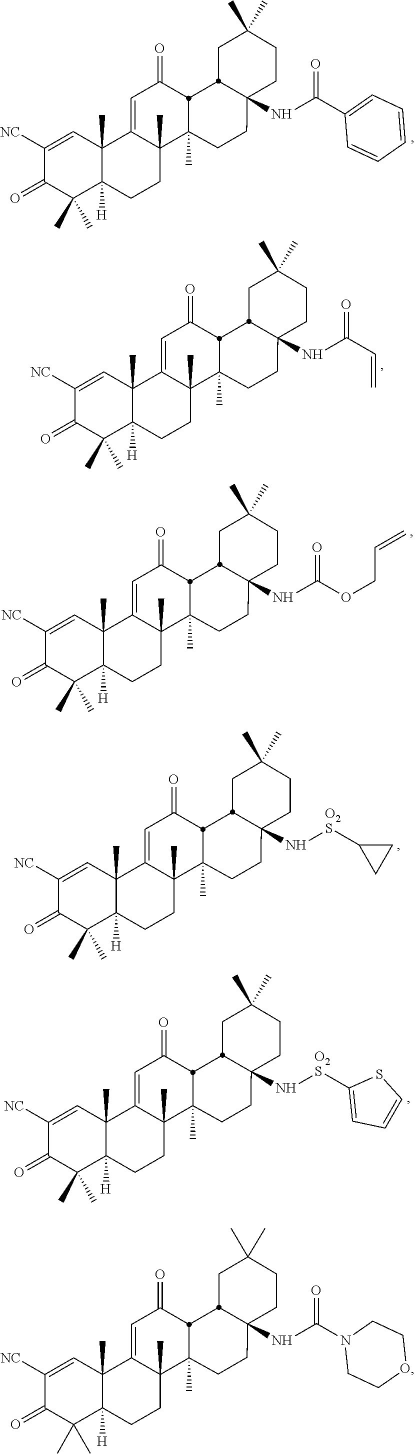

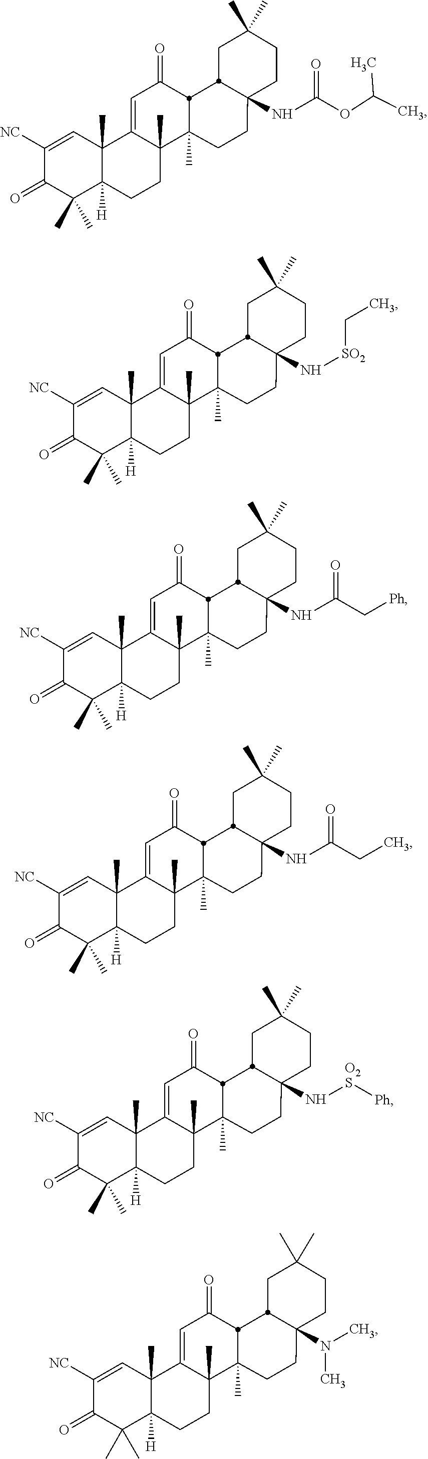

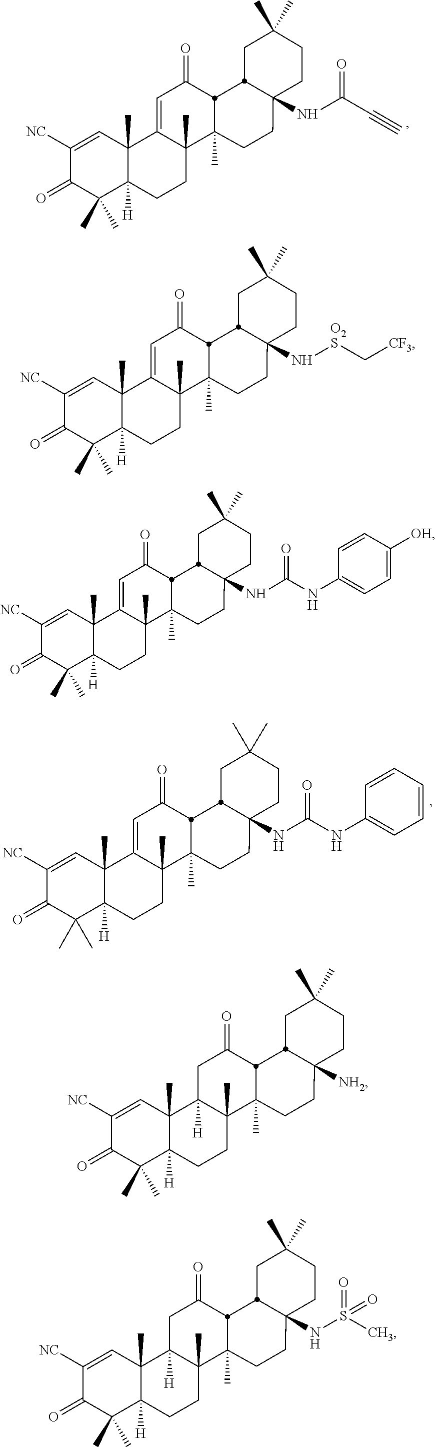

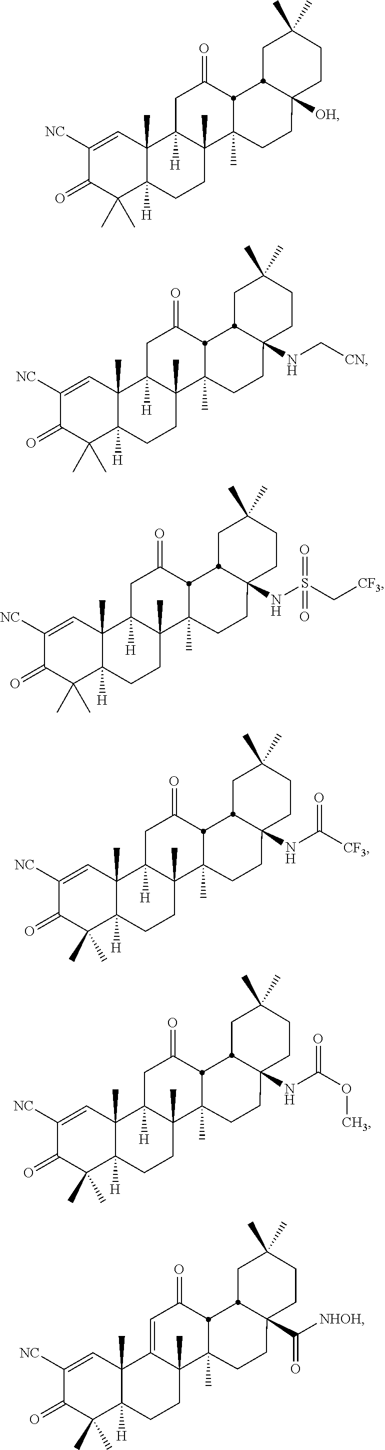

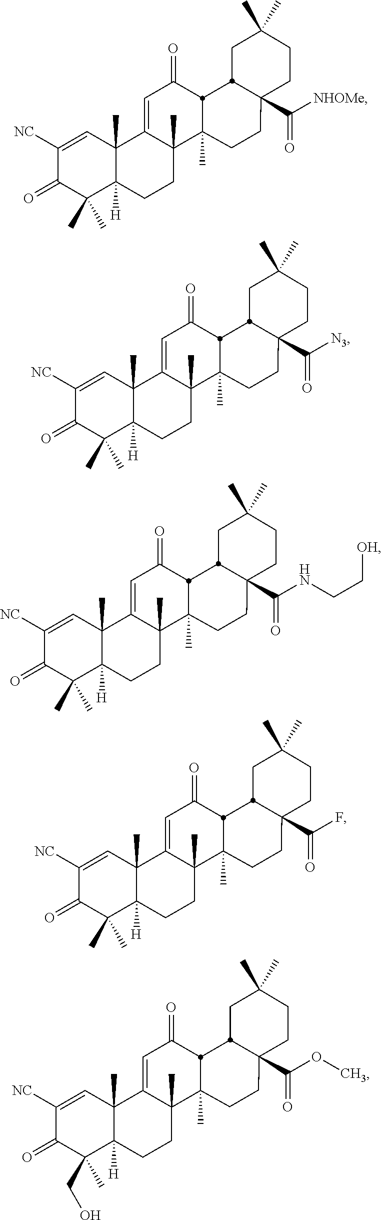

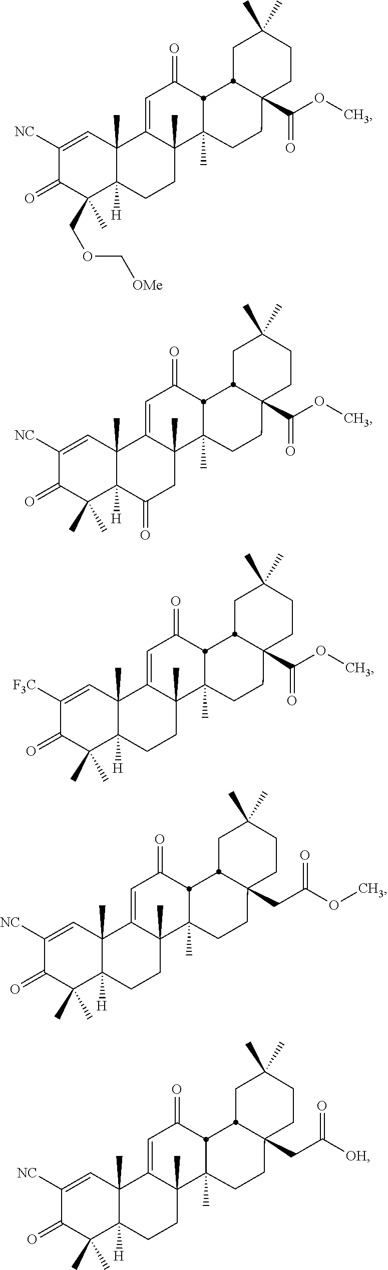

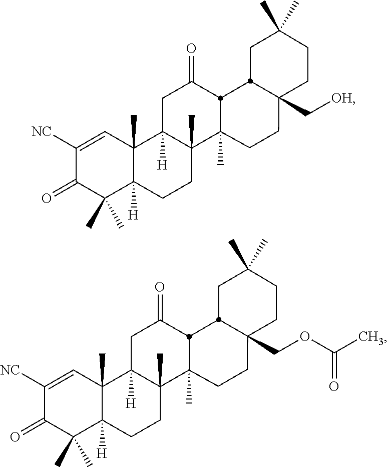

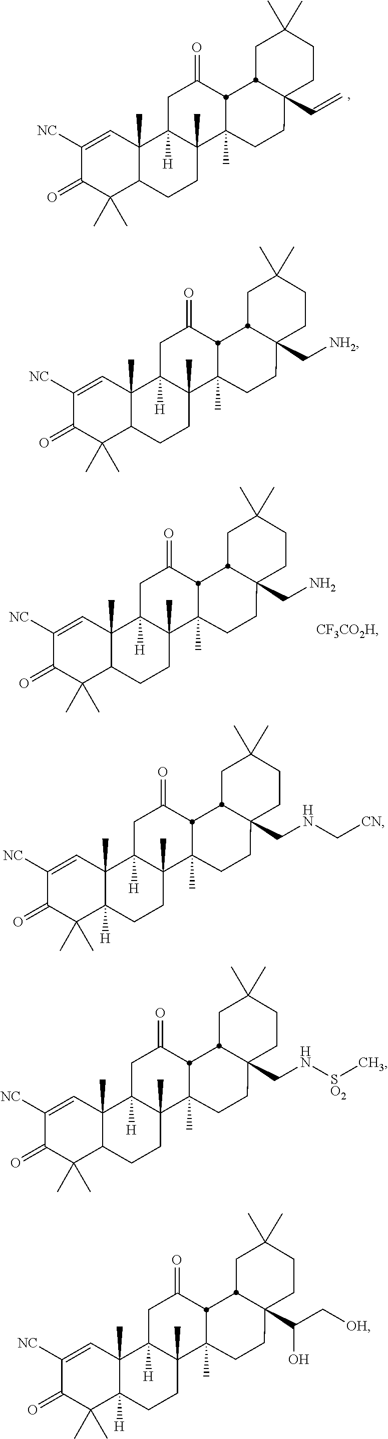

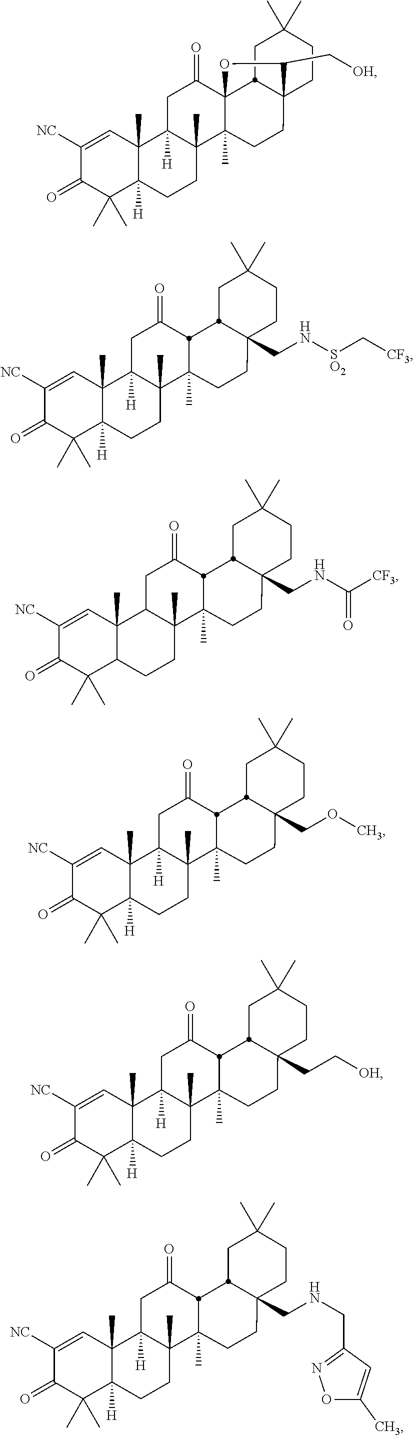

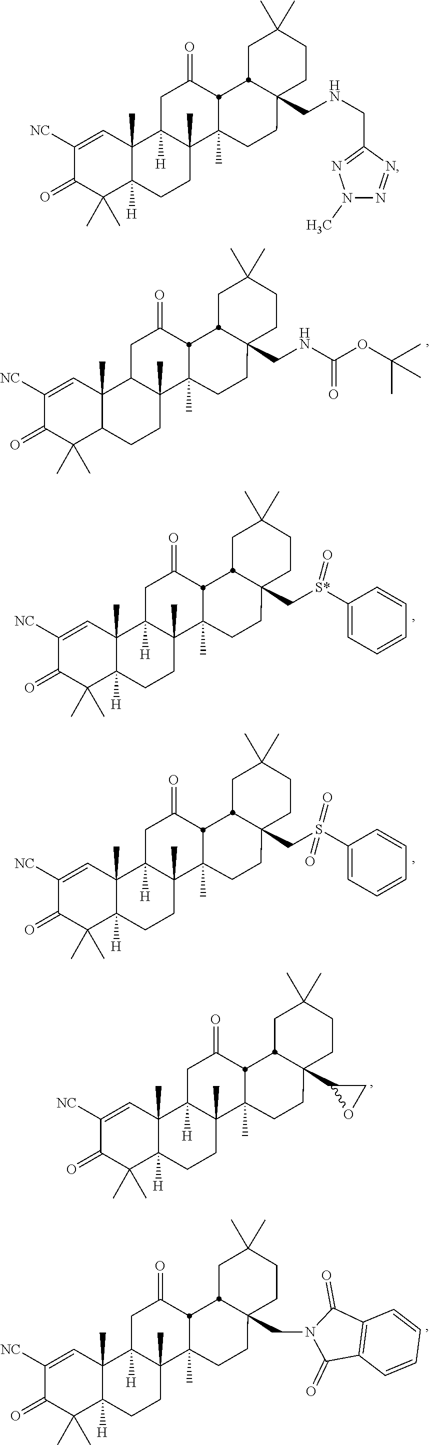

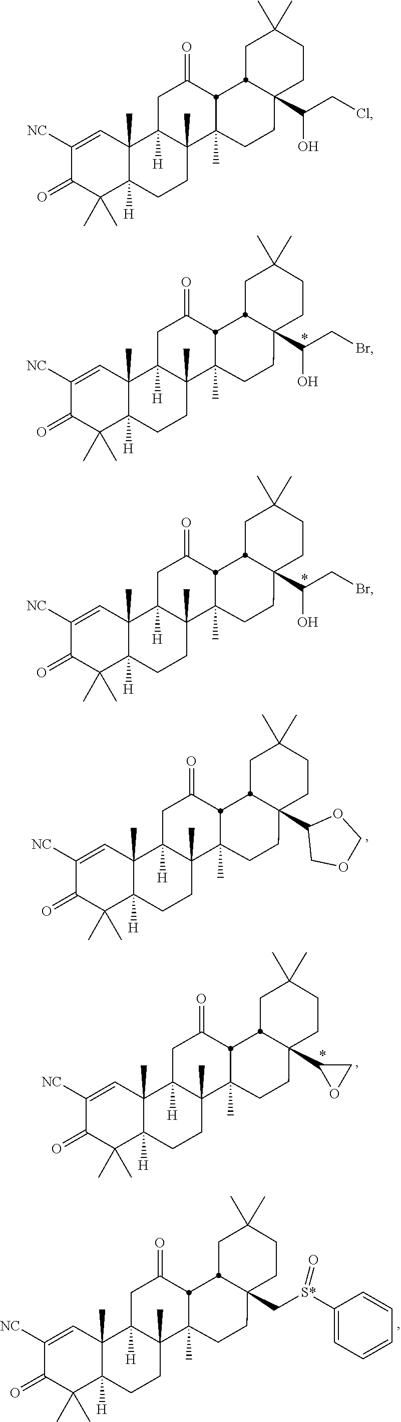

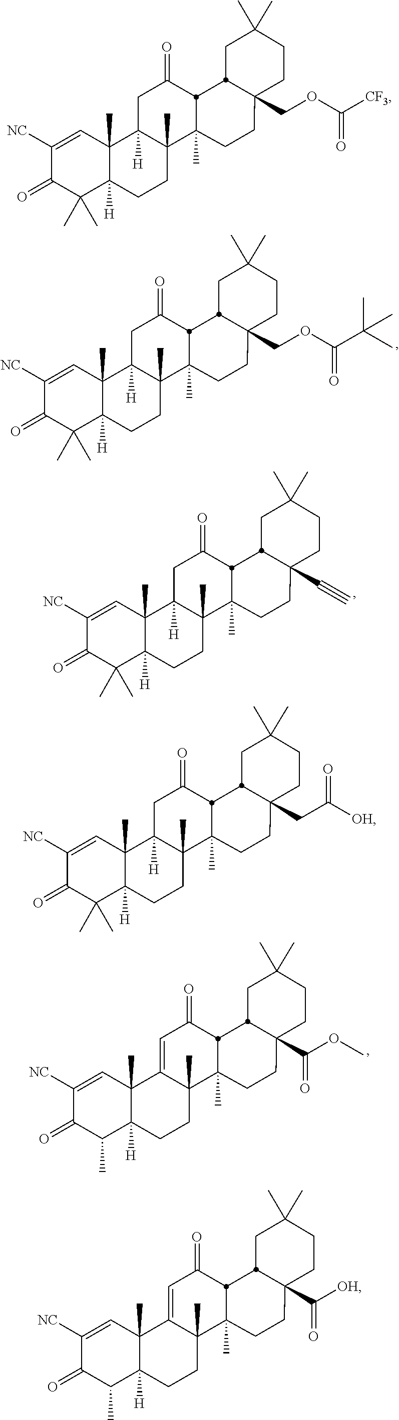

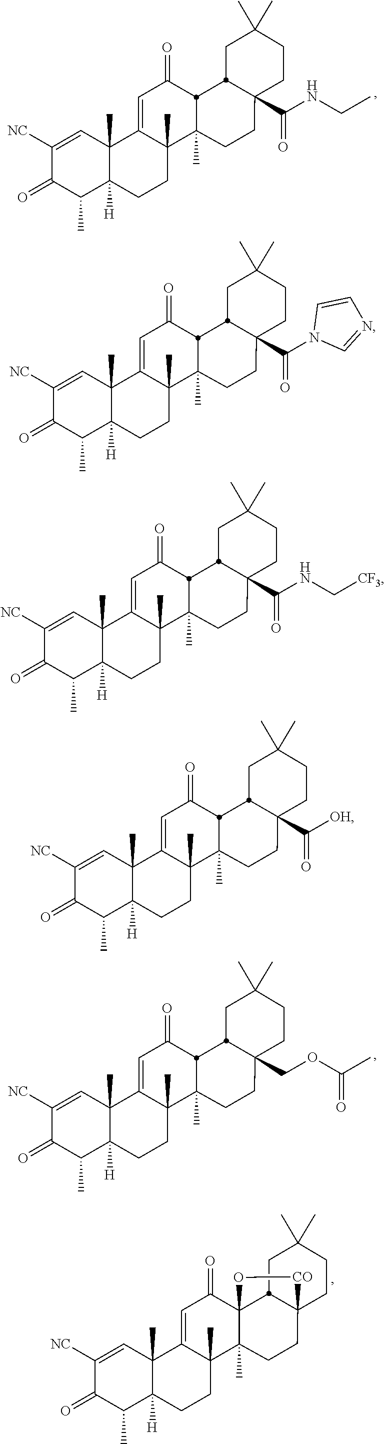

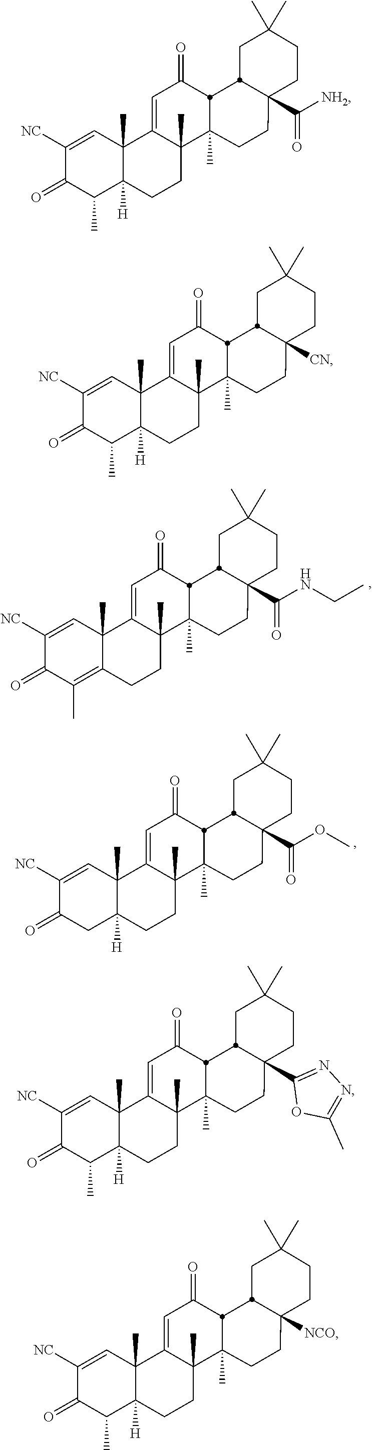

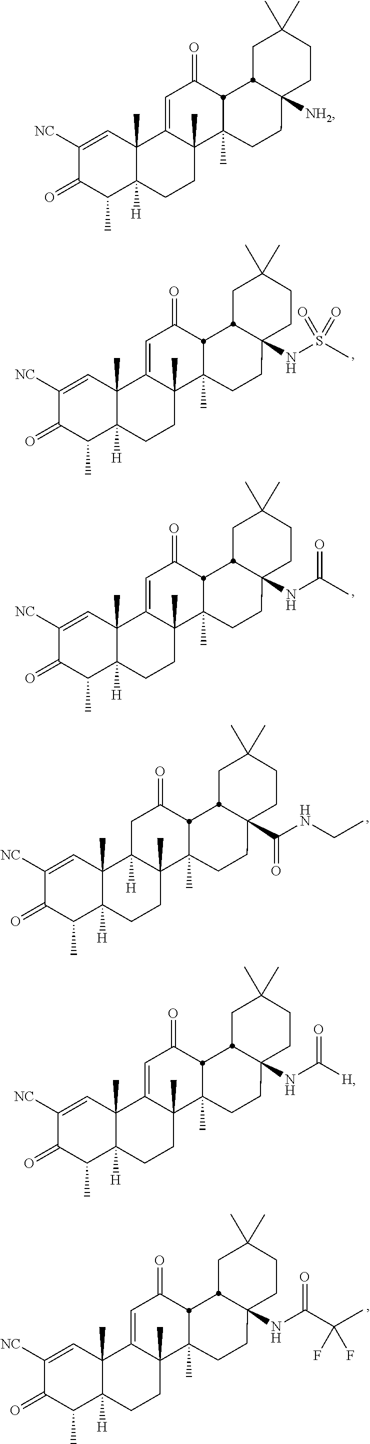

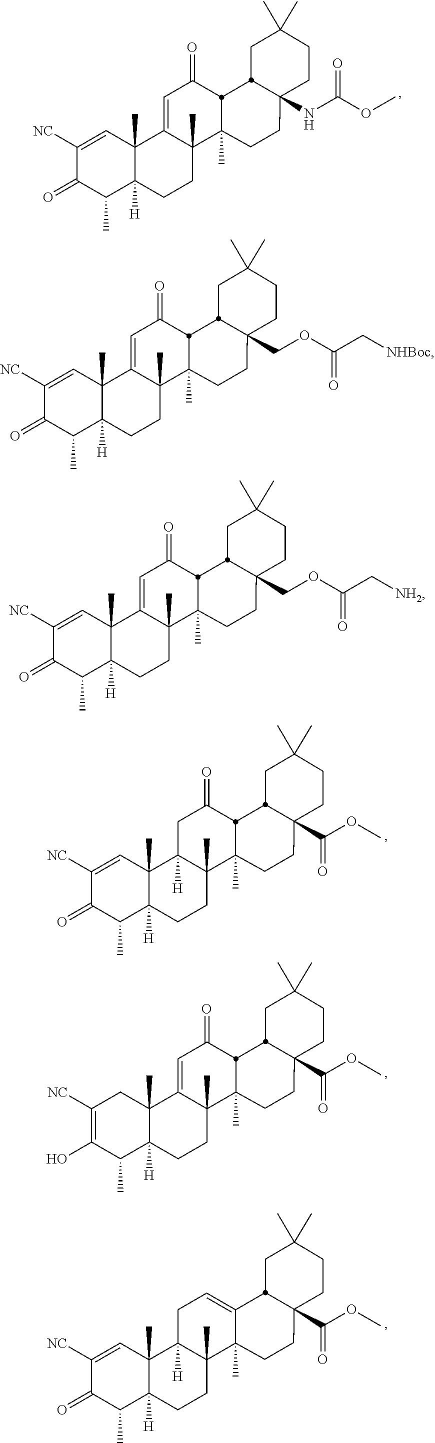

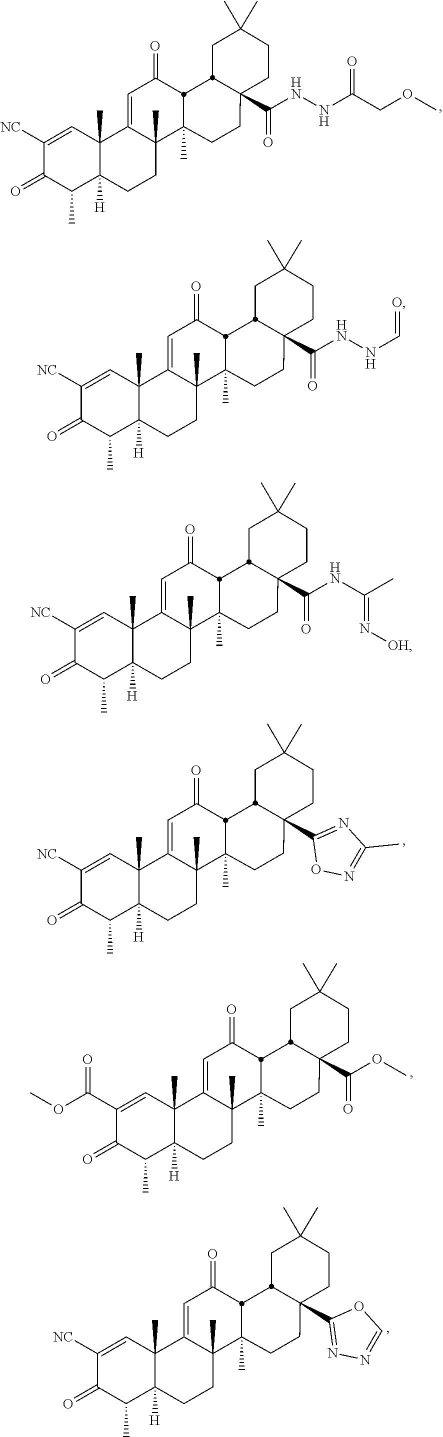

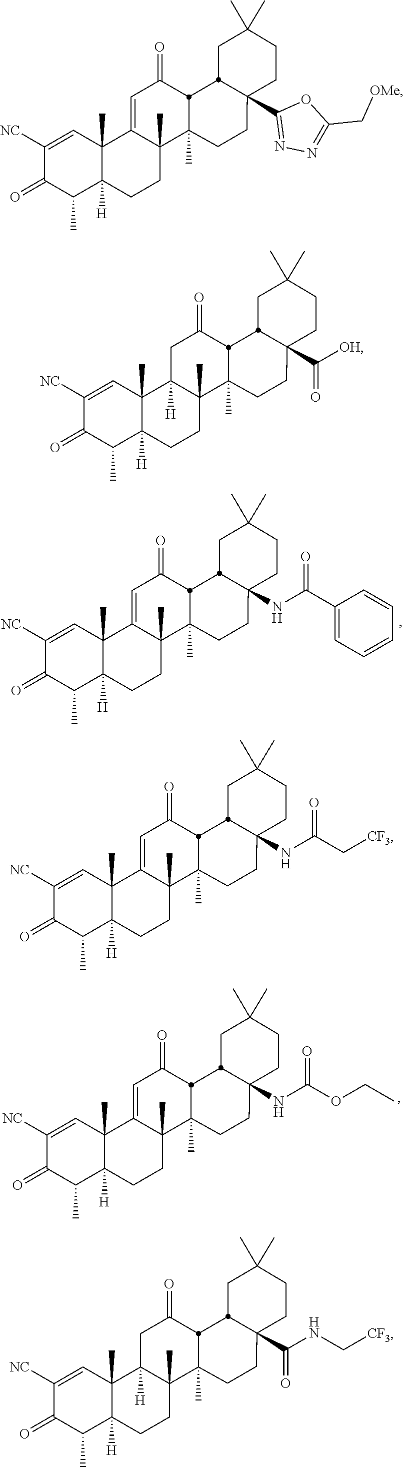

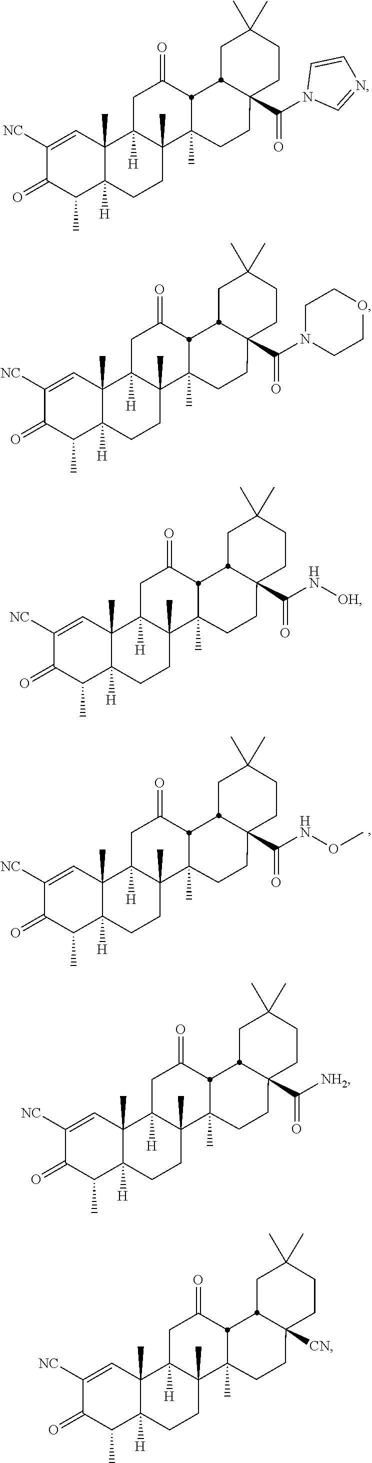

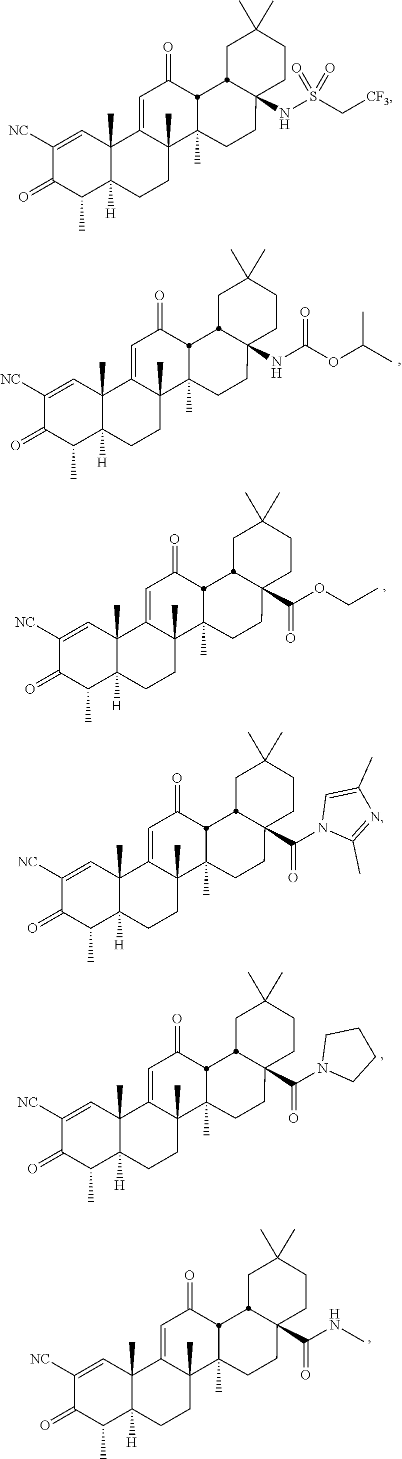

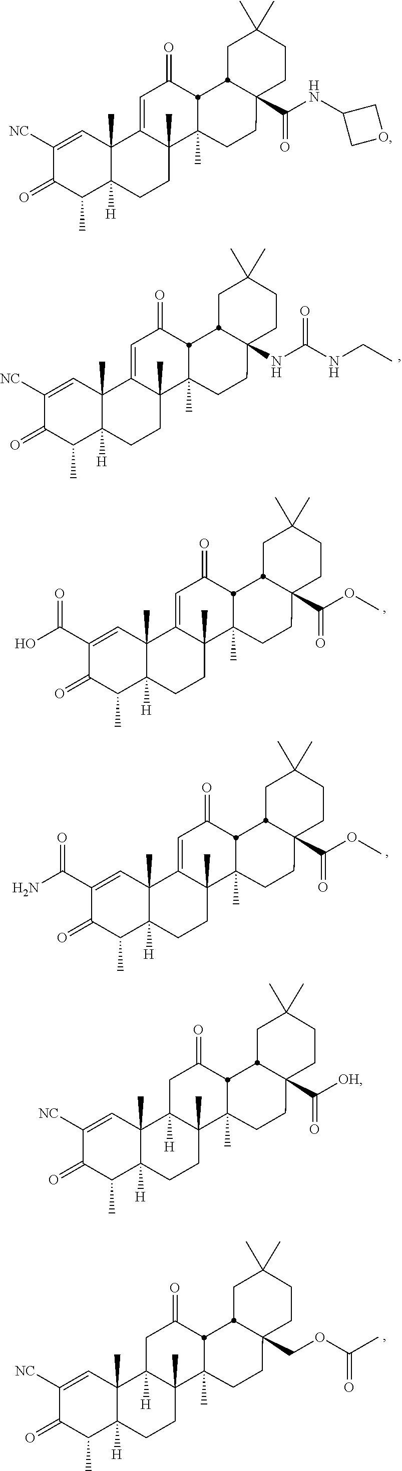

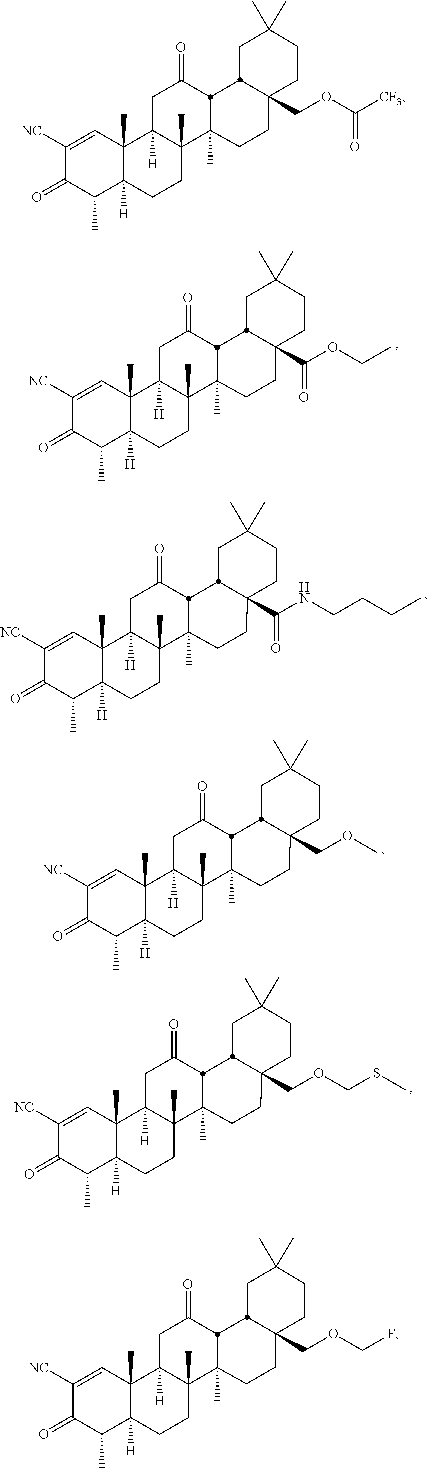

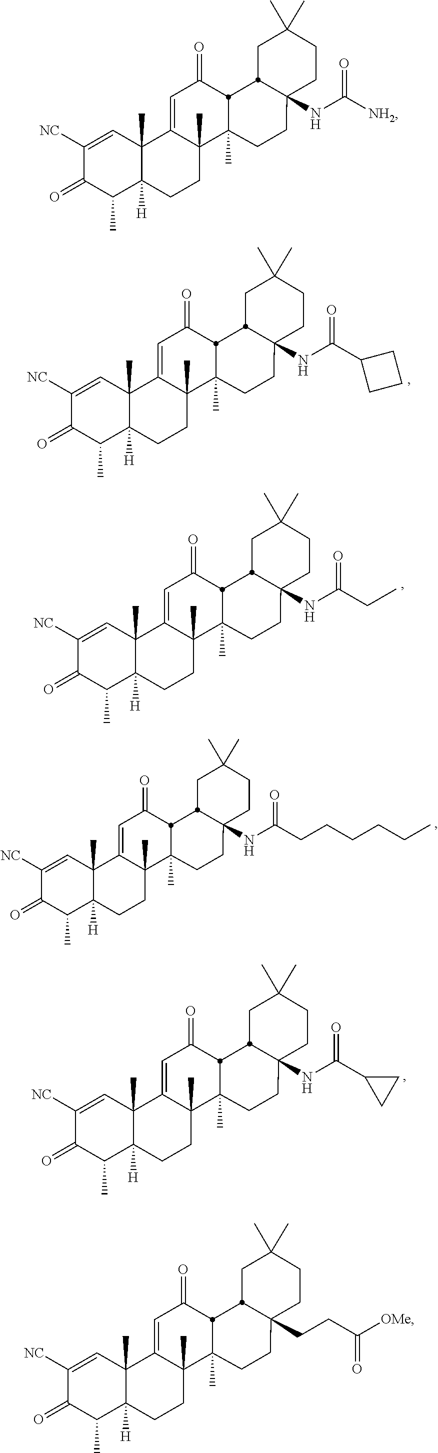

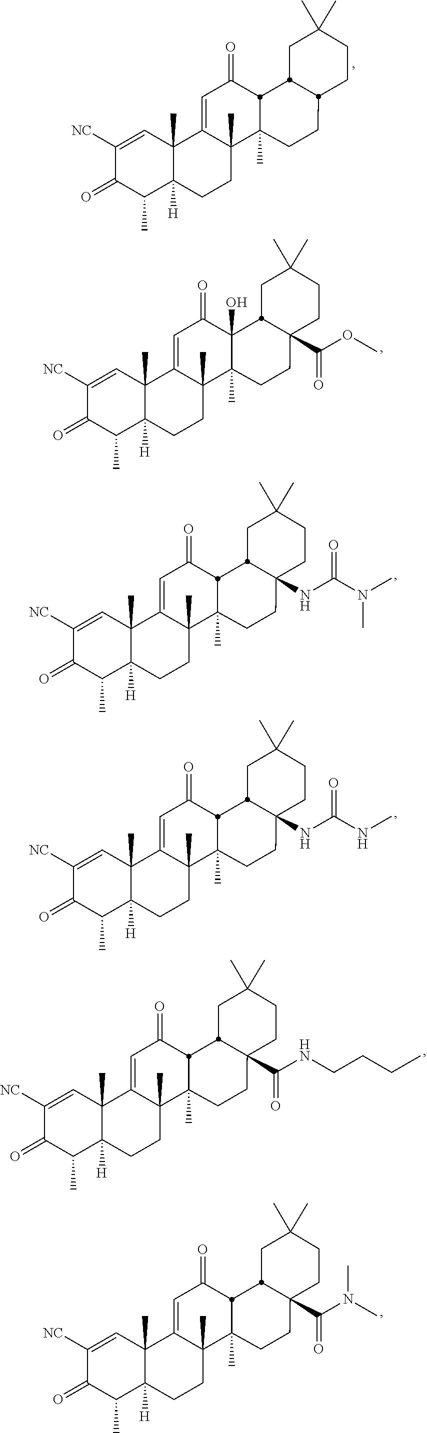

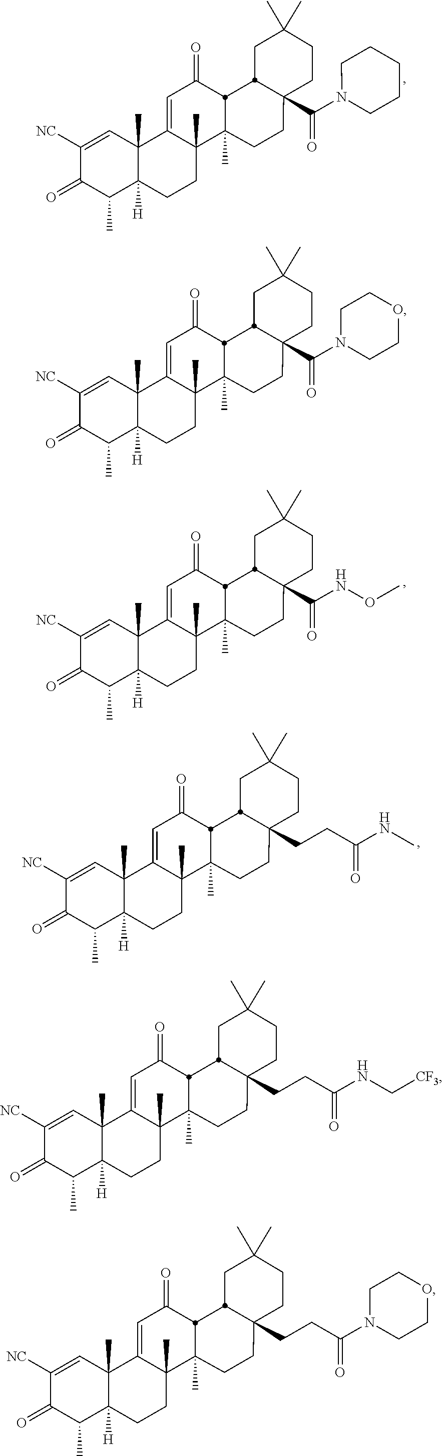

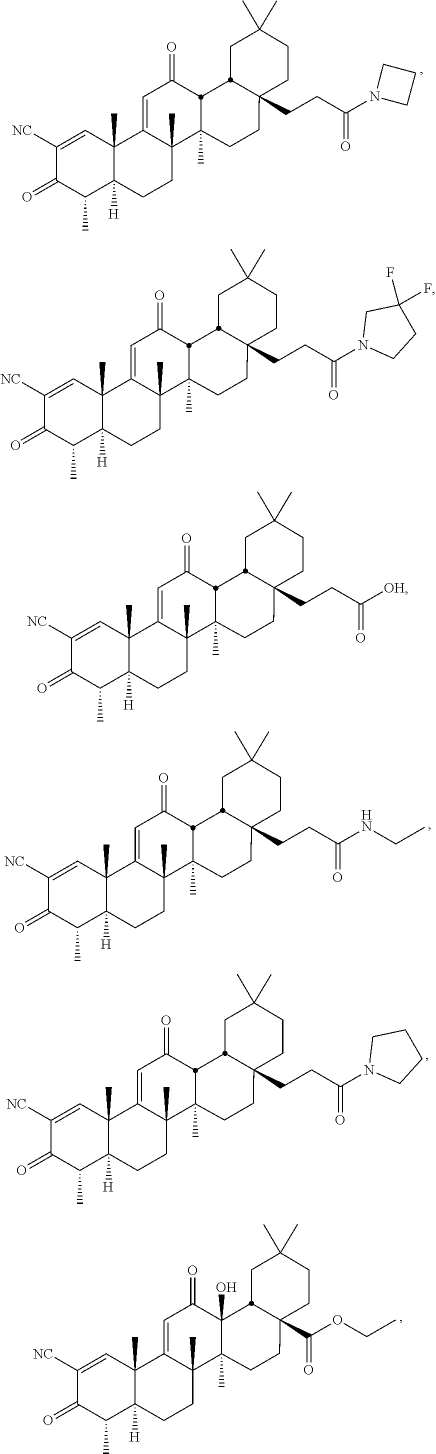

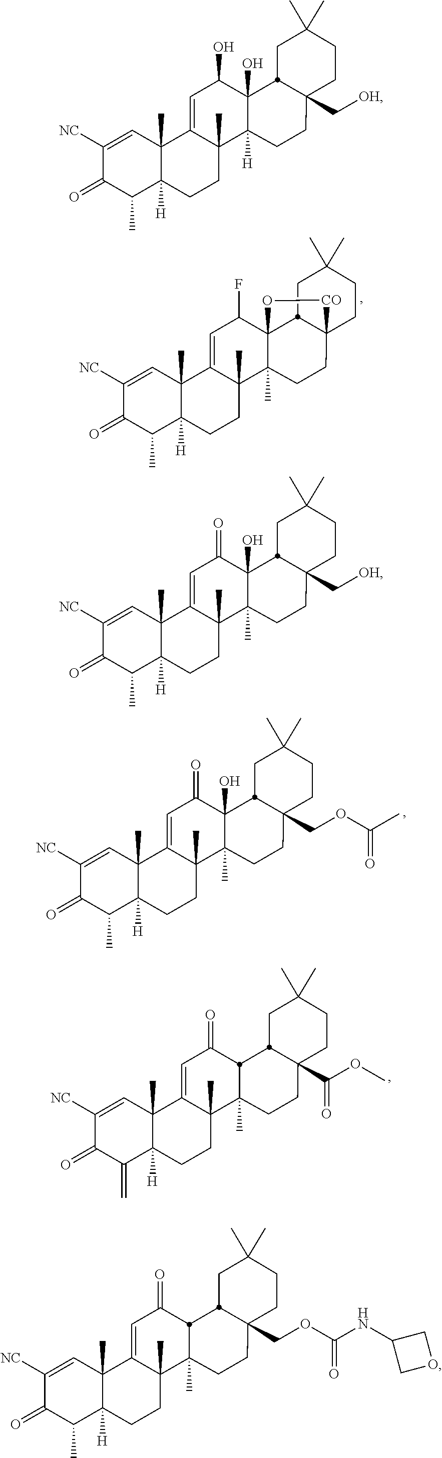

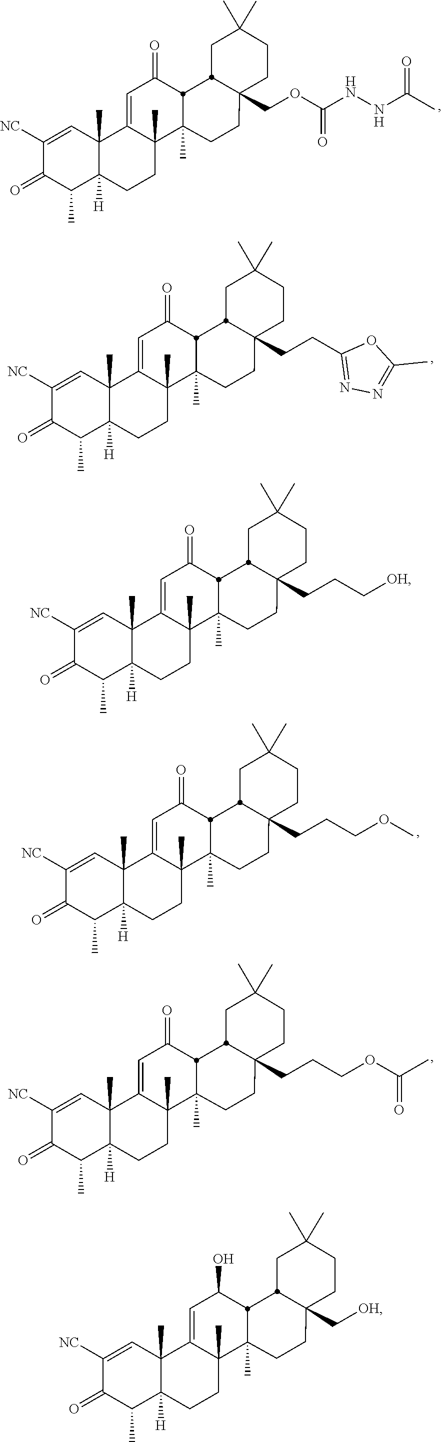

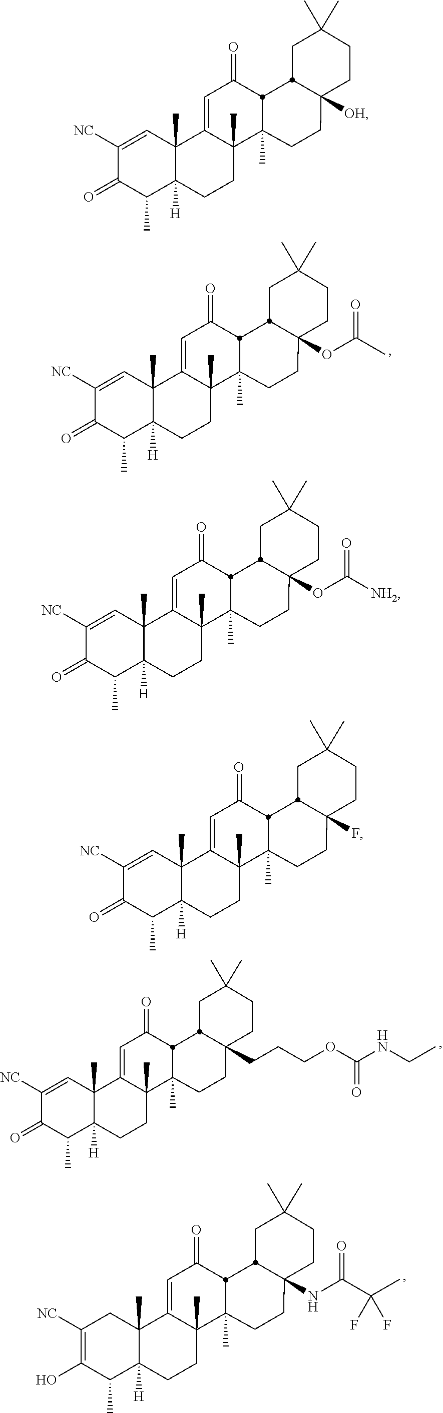

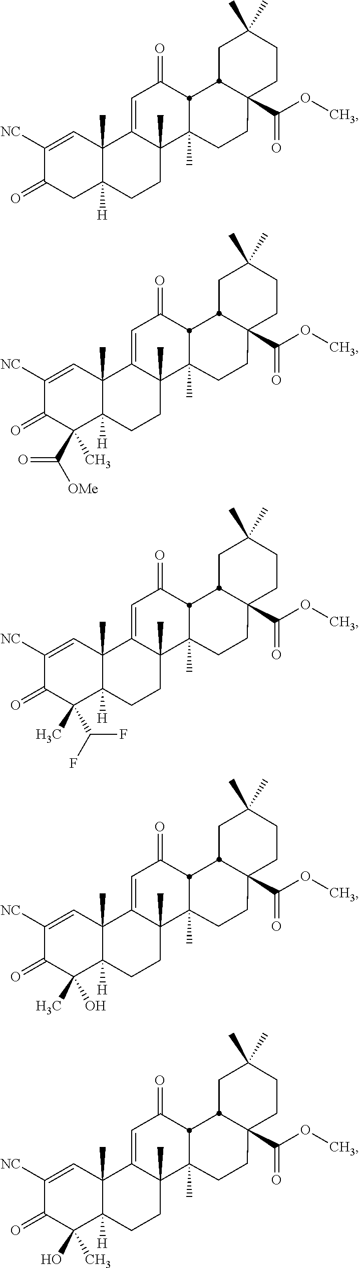

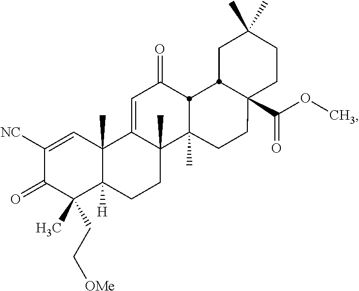

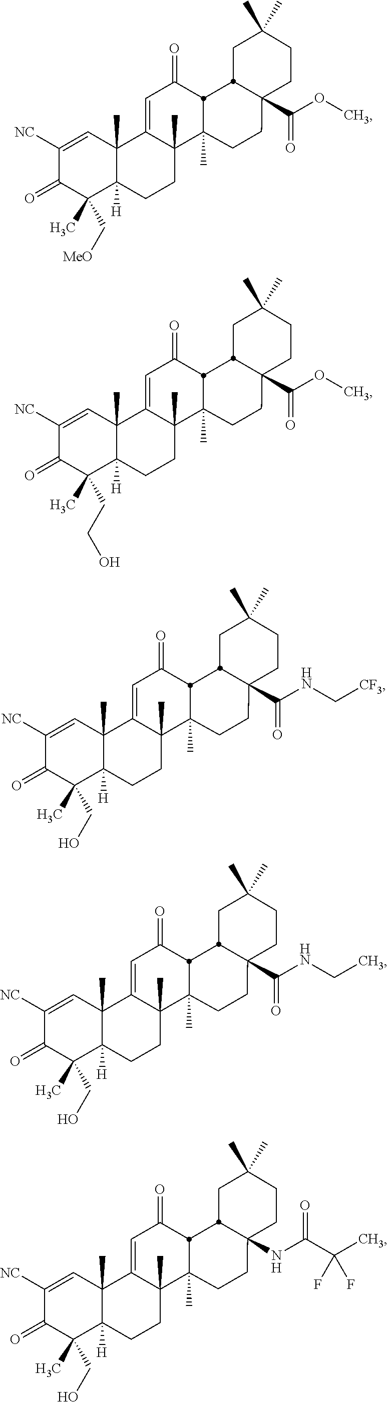

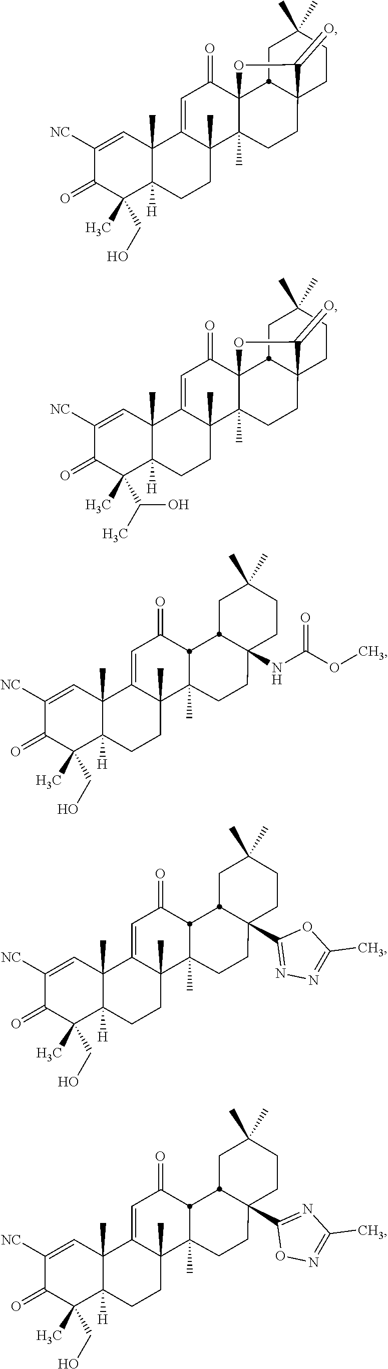

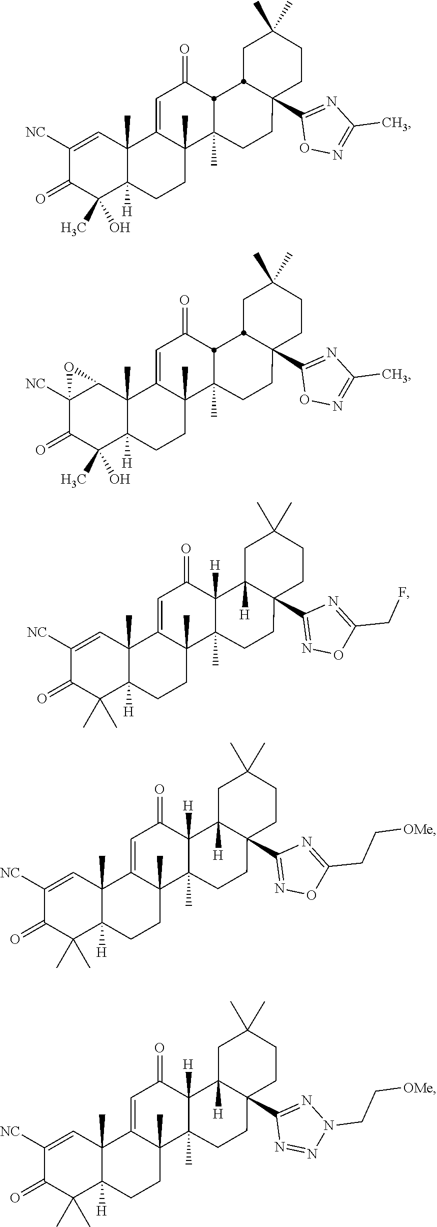

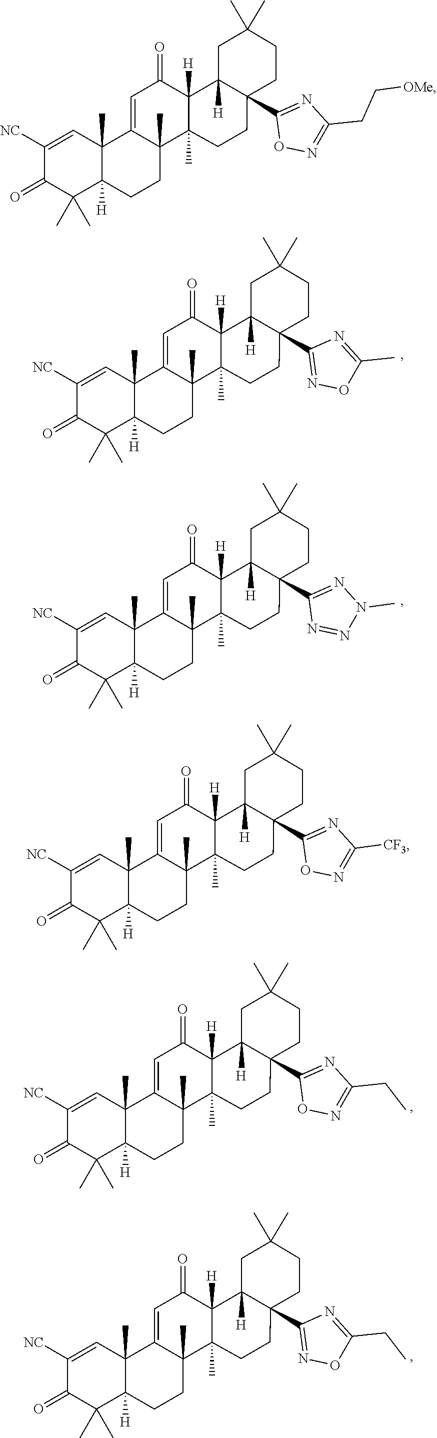

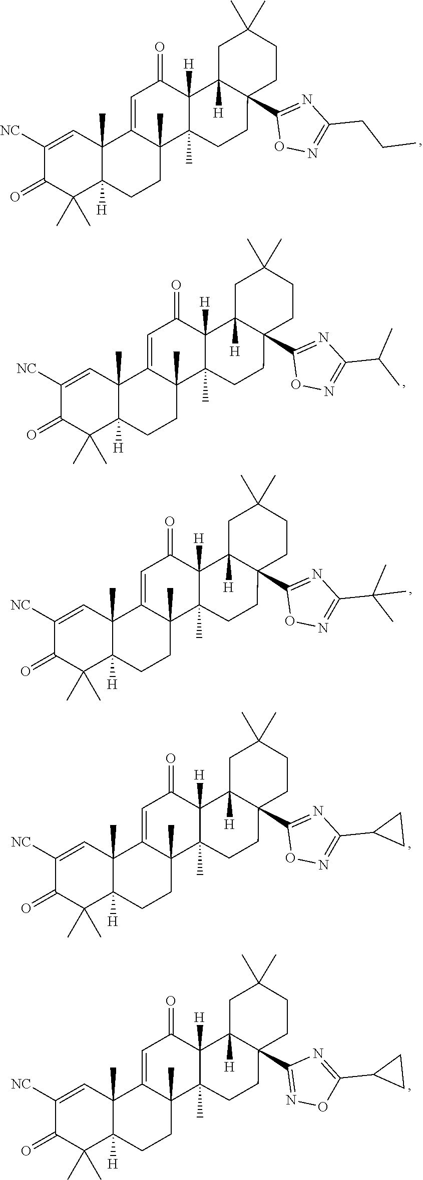

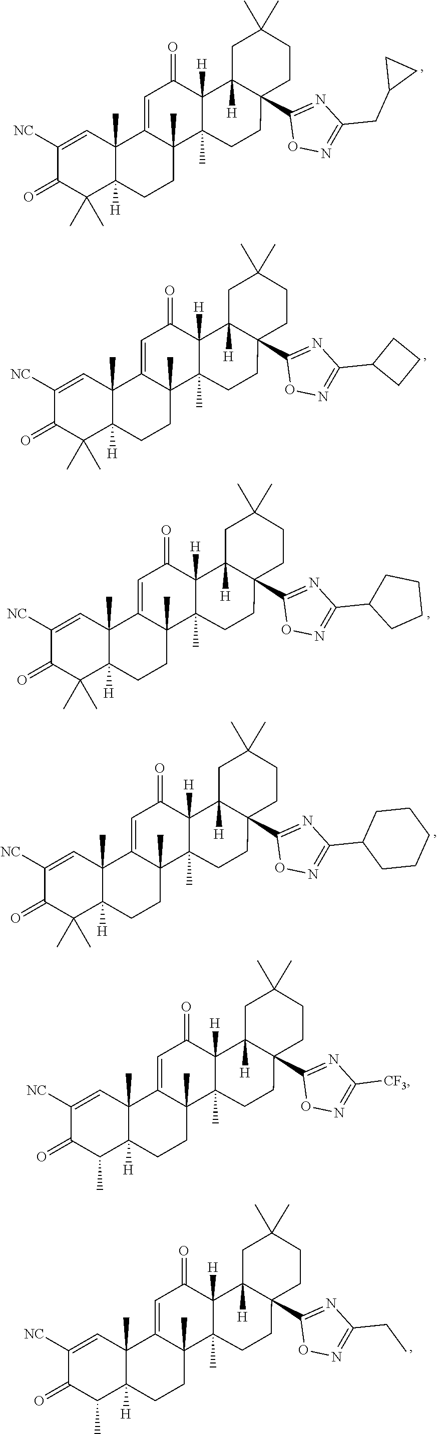

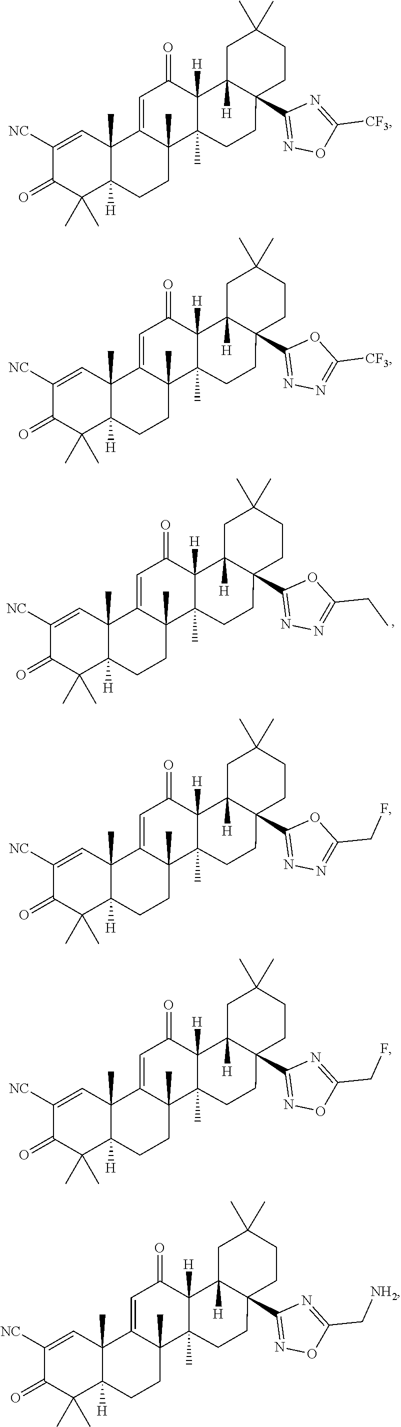

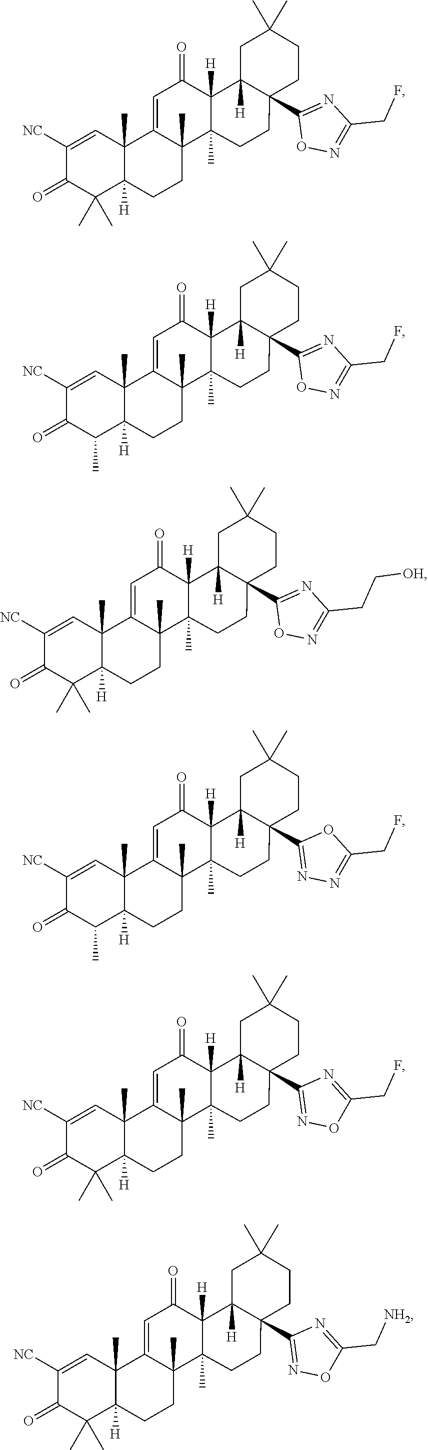

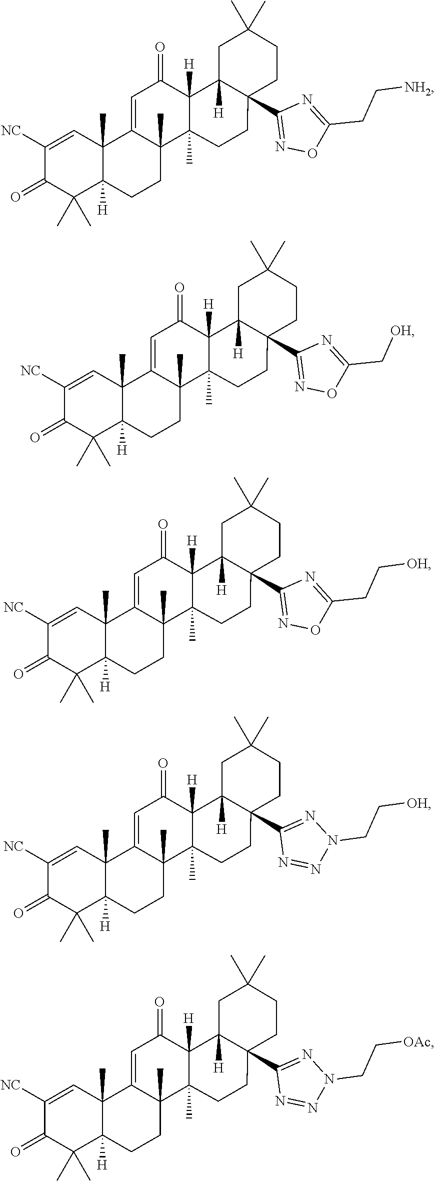

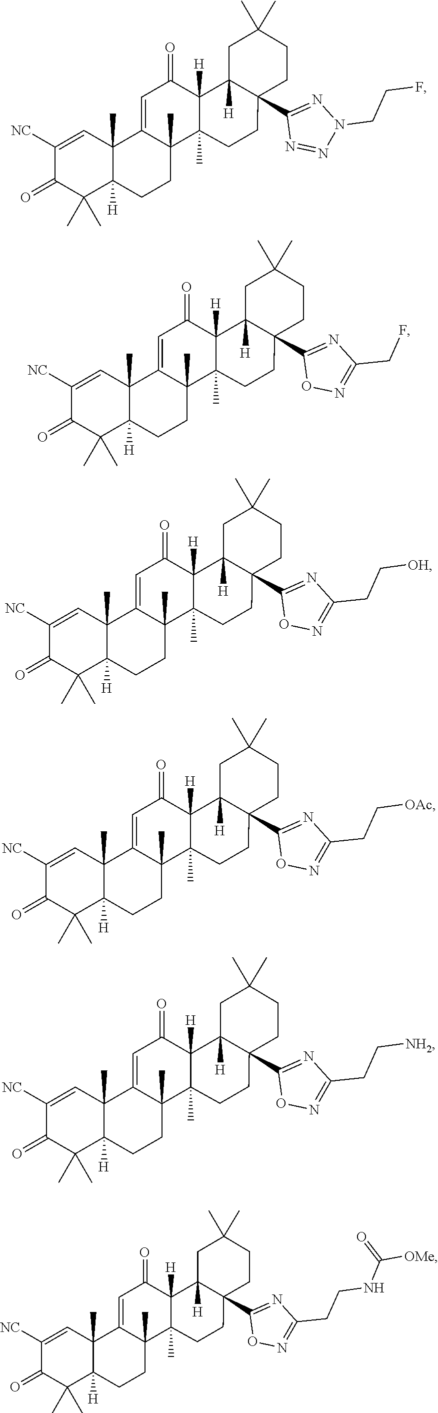

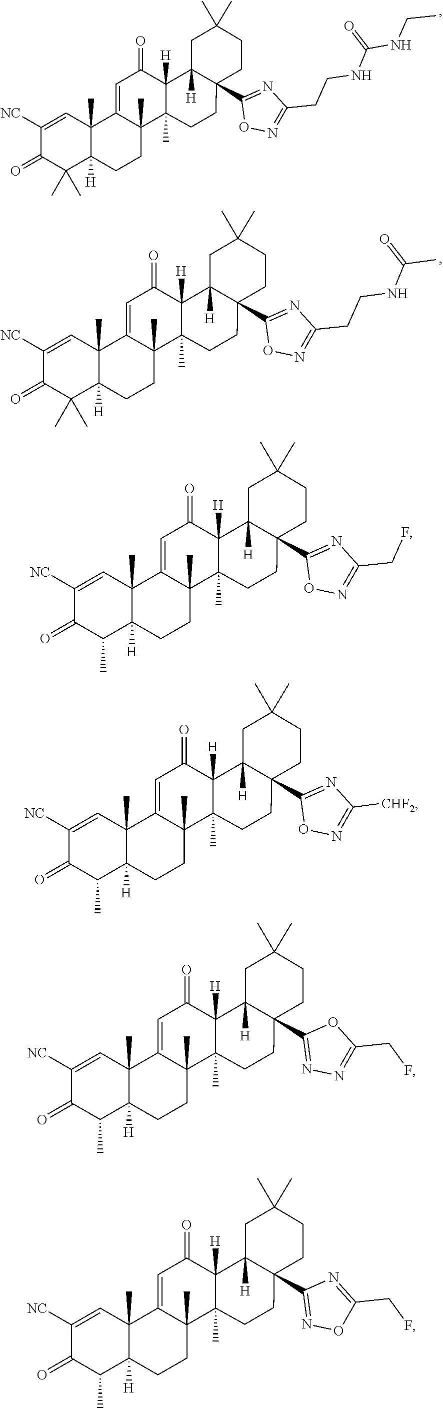

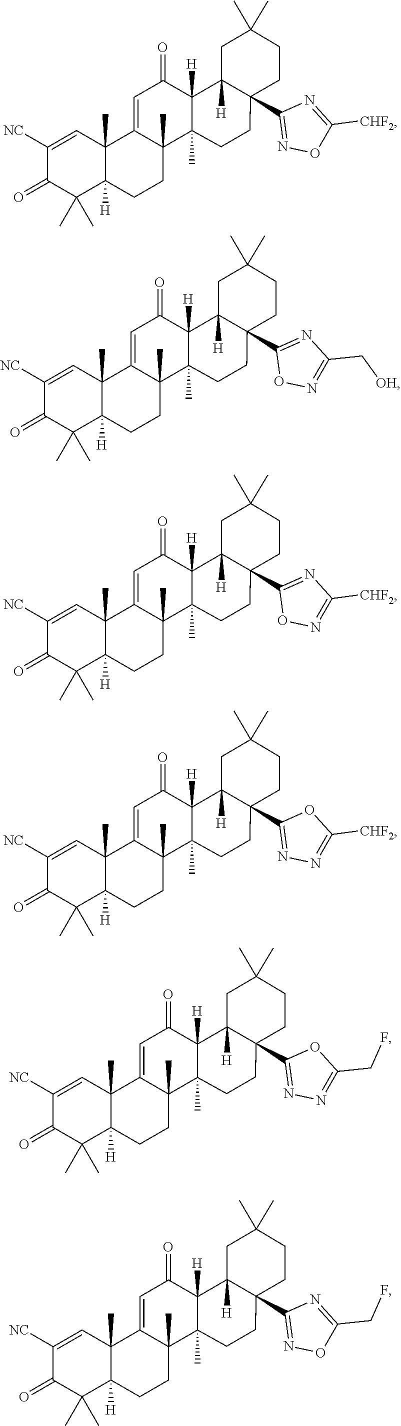

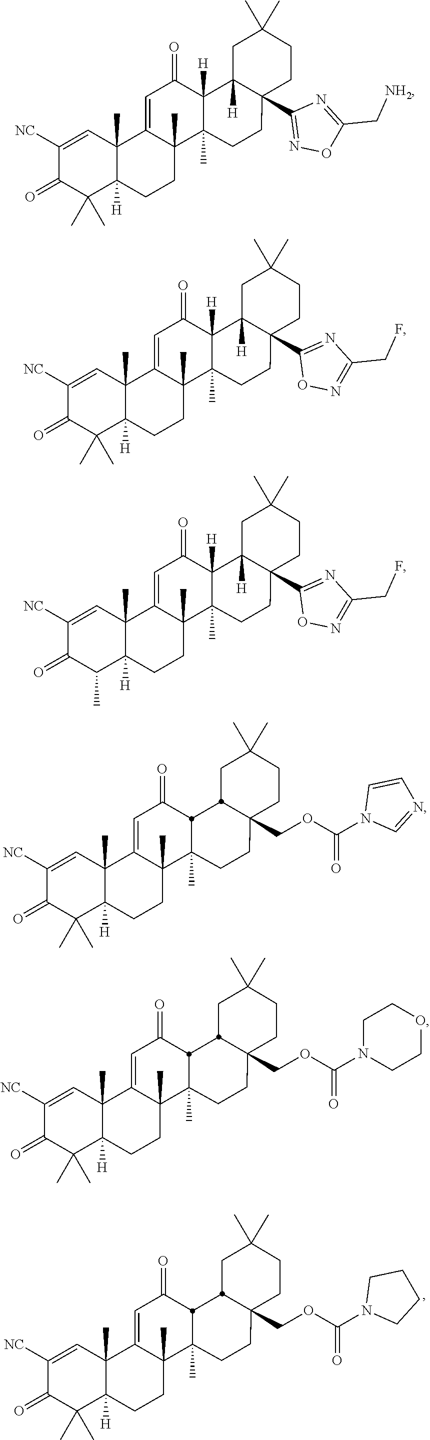

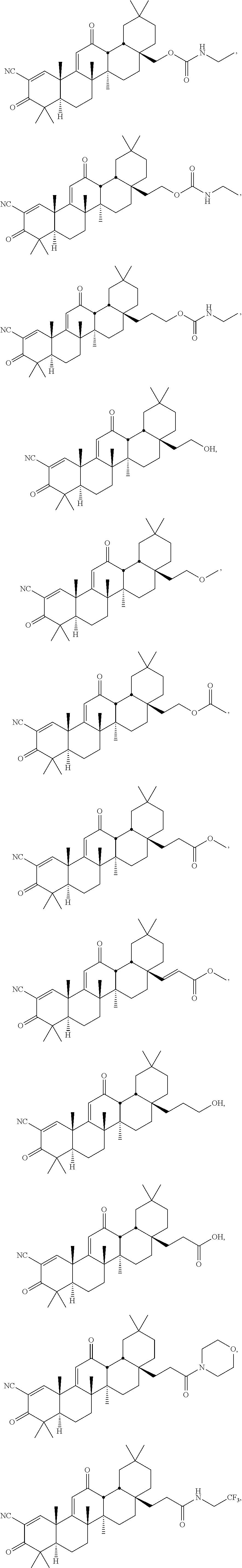

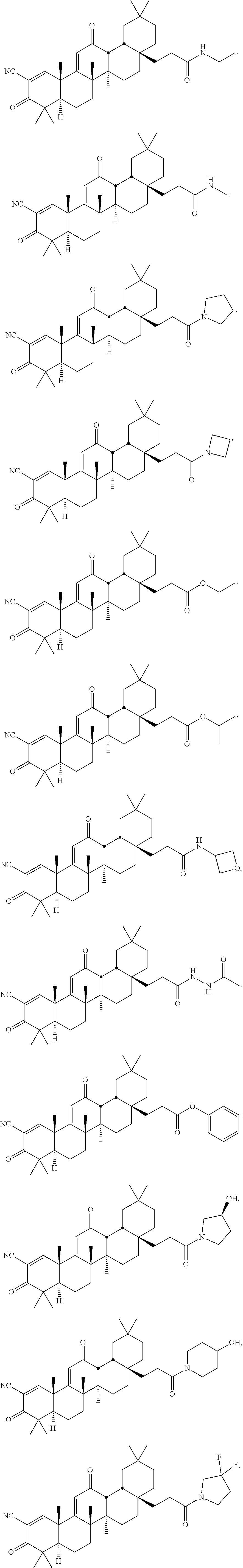

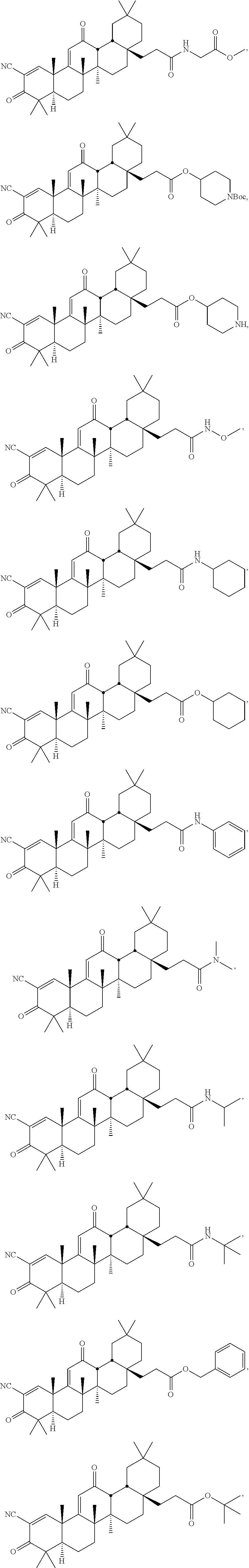

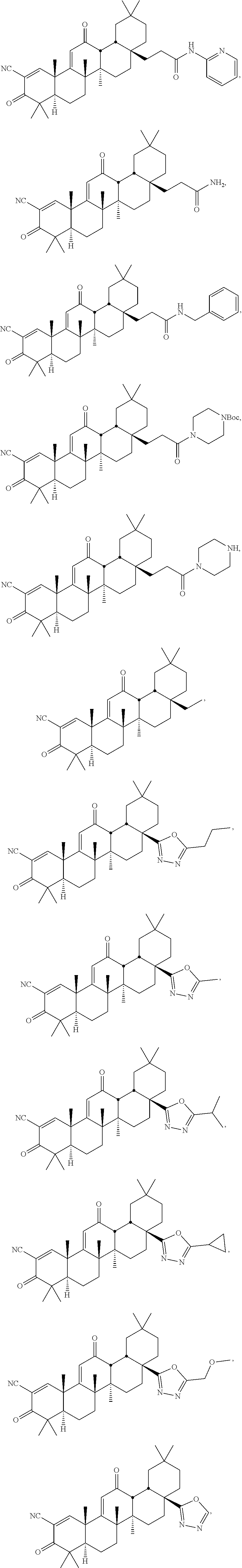

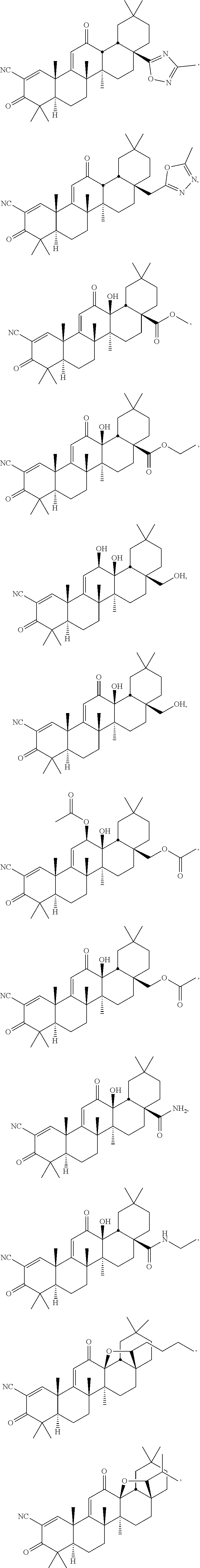

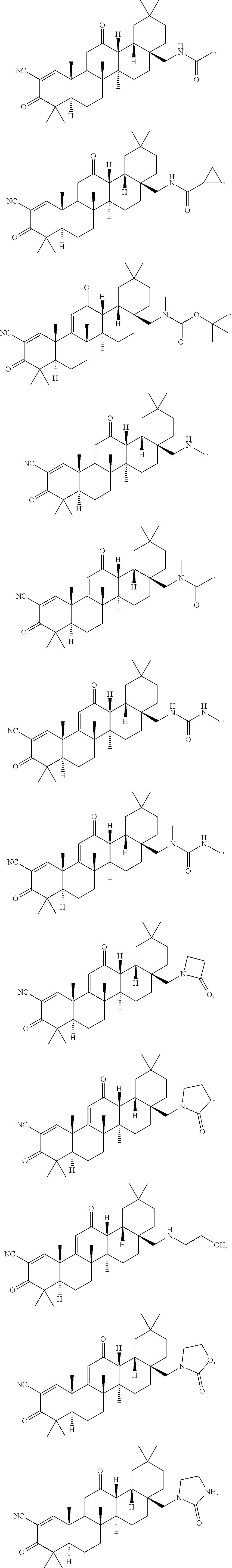

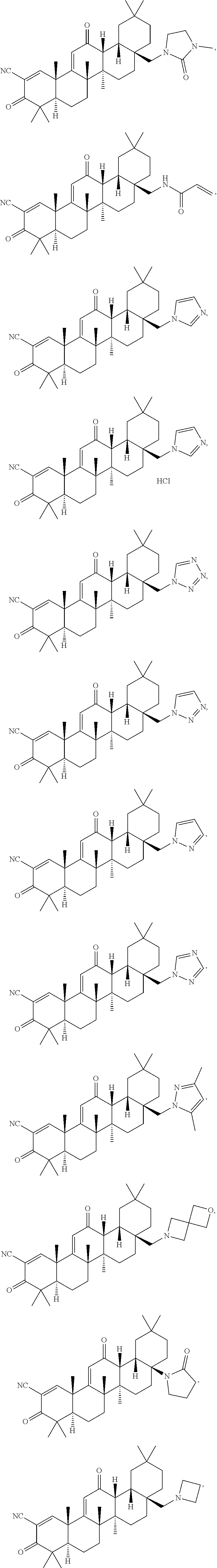

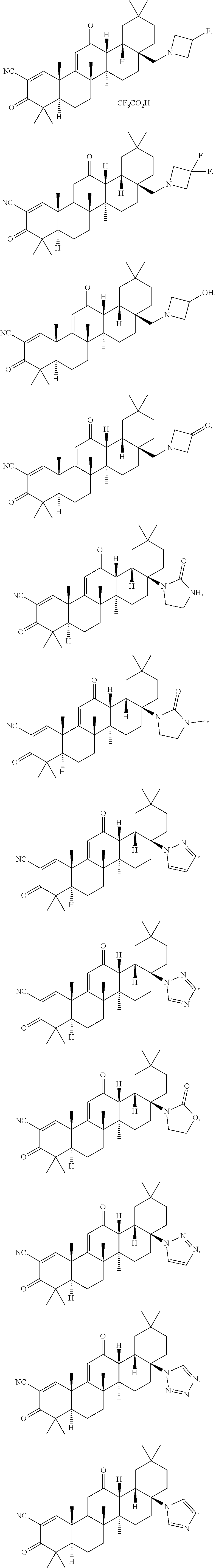

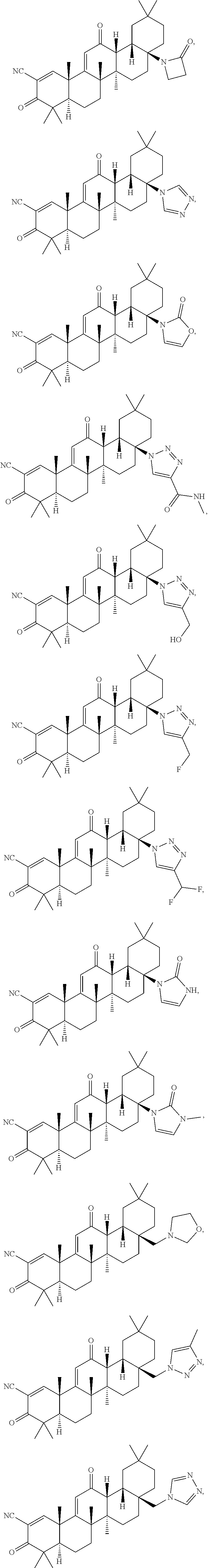

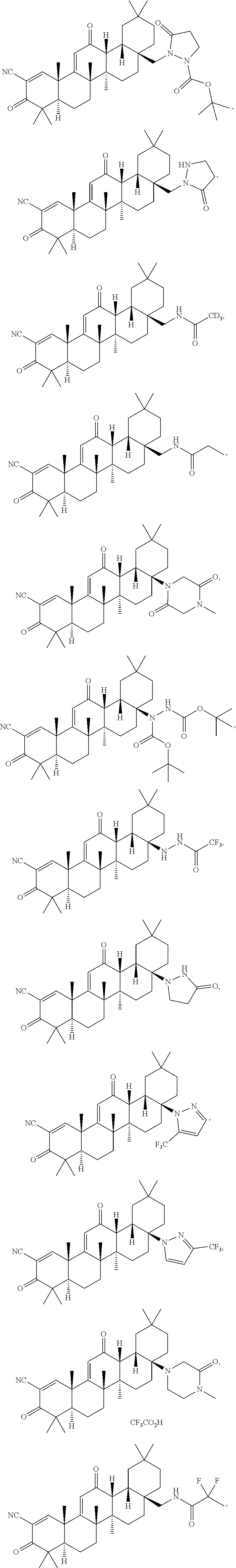

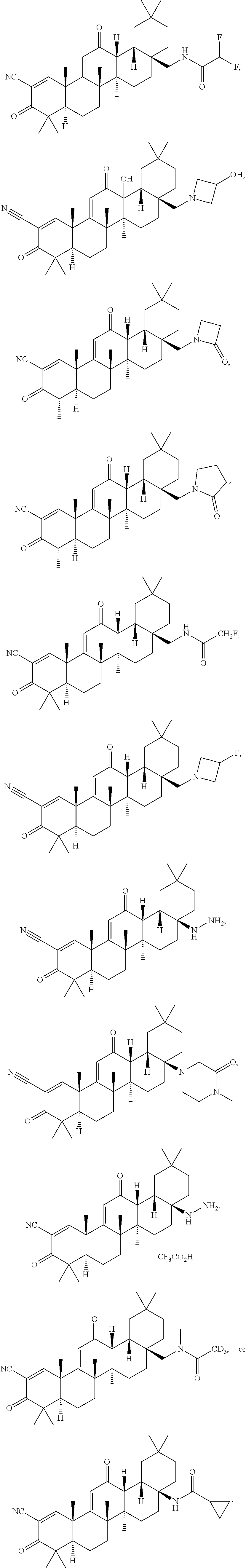

These compounds are known as antioxidant inflammation modulators. These compounds have shown the ability to activate Nrf2, as measured by elevated expression of one or more Nrf2 target genes (e.g., NQO1 or HO-1; Dinkova-Kostova et al., 2005). Further, these compounds are capable of indirect and direct inhibition of pro-inflammatory transcription factors including NF-κB and STAT3 (Ahmad et al., 2006; Ahmad et al., 2008).

Compounds employed may be made using the methods described by Honda et al. (2000a); Honda et al. (2000b); Honda et al. (2002); and U.S. patent Application Publications 2009/0326063, 2010/0056777, 2010/0048892, 2010/0048911, 2010/0041904, 2003/0232786, 2008/0261985 and 2010/0048887, all of which are incorporated by reference herein. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure (2013), which is incorporated by reference herein. In addition, the synthetic methods may be further modified and optimized for preparative, pilot- or large-scale production, either batch of continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

Compounds of the present invention may contain one or more asymmetrically substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Polymorphic forms of the compounds of the present invention may be used in accordance with the methods of this inventions.

Compounds employed in methods of the invention may also exist in prodrug form. Since prodrugs enhance numerous desirable qualities of pharmaceuticals, e.g., solubility, bioavailability, manufacturing, etc., the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject or patient, cleaves to form a hydroxy, amino, or carboxylic acid, respectively.

It should be recognized that the particular anion or cation forming a part of any salt of this invention is not critical, so long as the salt, as a whole, is pharmacologically acceptable. Examples of pharmaceutically acceptable salts and their methods of preparation and use are presented in Handbook of Pharmaceutical Salts: Properties, and Use (2002), which is incorporated herein by reference.

In some embodiments, the compounds employed in the methods described in the present invention have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

### A. Bardoxolone Methyl

Bardoxolone methyl (RTA 402; BARD; CDDO-Me), an antioxidant inflammation modulator (AIM), suppresses the induction of several important inflammatory mediators, such as iNOS, COX-2, TNFα, and IFNγ, in activated macrophages, thereby restoring redox homeostasis in inflamed tissues. RTA 402 has also been reported to activate the Keap1/Nrf2/ARE signaling pathway resulting in the production of several anti-inflammatory and antioxidant proteins, such as heme oxygenase-1 (HO-1). It induces the cytoprotective transcription factor Nrf2 and suppresses the activities of the pro-oxidant and pro-inflammatory transcription factors NF-κB and STAT3. In vivo, RTA 402 has demonstrated significant single agent anti-inflammatory activity in several animal models of inflammation such as renal damage in the cisplatin model and acute renal injury in the ischemia-reperfusion model. In addition, significant reductions in serum creatinine have been observed in patients treated with RTA 402.

Polymorphic forms of the compounds of the present invention, e.g., Forms A and B of CDDO-Me, may be used in accordance with the methods of this inventions. Form B displays a bioavailability that is surprisingly better than that of Form A. Specifically the bioavailability of Form B was higher than that of Form A CDDO-Me in monkeys when the monkeys received equivalent dosages of the two forms orally, in gelatin capsules. See U.S. Patent Application Publication 2009/0048204, which is incorporated by reference herein in its entirety.

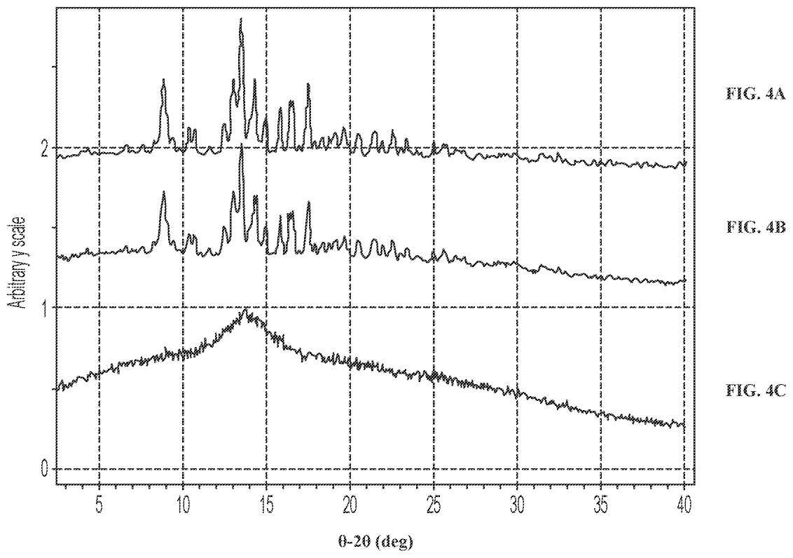

“Form A” of CDDO-Me (RTA 402) is unsolvated (non-hydrous) and can be characterized by a distinctive crystal structure, with a space group of P4₃ 2₁² (no. 96) unit cell dimensions of a=14.2 Å, b=14.2 Å and c=81.6 Å, and by a packing structure, whereby three molecules are packed in helical fashion down the crystallographic b axis. In some embodiments, Form A can also be characterized by X-ray powder diffraction (XRPD) pattern (CuKα) comprising significant diffraction peaks at about 8.8, 12.9, 13.4, 14.2 and 17.4° 0. In some variations, the X-ray powder diffraction of Form A is substantially as shown in FIG. 4A or FIG. 4B.

Unlike Form A, “Form B” of CDDO-Me is in a single phase but lacks such a defined crystal structure. Samples of Form B show no long-range molecular correlation, i.e., above roughly 20 Å. Moreover, thermal analysis of Form B samples reveals a glass transition temperature (T_{g}) in a range from about 120° C. to about 130° C. In contrast, a disordered nanocrystalline material does not display a T_{g} but instead only a melting temperature (Tm), above which crystalline structure becomes a liquid. Form B is typified by an XRPD spectrum (FIG. 4C) differing from that of Form A (FIG. 4A or FIG. 4B). Since it does not have a defined crystal structure, Form B likewise lacks distinct XRPD peaks, such as those that typify Form A, and instead is characterized by a general “halo” XRPD pattern. In particular, the non-crystalline Form B falls into the category of “X-ray amorphous” solids because its XRPD pattern exhibits three or fewer primary diffraction halos. Within this category, Form B is a “glassy” material.

Form A and Form B of CDDO-Me are readily prepared from a variety of solutions of the compound. For example, Form B can be prepared by fast evaporation or slow evaporation in MTBE, THF, toluene, or ethyl acetate. Form A can be prepared in several ways, including via fast evaporation, slow evaporation, or slow cooling of a CDDO-Me solution in ethanol or methanol. Preparations of CDDO-Me in acetone can produce either Form A, using fast evaporation, or Form B, using slow evaporation.

Various means of characterization can be used together to distinguish Form A and Form B CDDO-Me from each other and from other forms of CDDO-Me. Illustrative of the techniques suitable for this purpose are solid state Nuclear Magnetic Resonance (NMR), X-ray powder diffraction (compare FIGS. 4A & B with FIG. 4C), X-ray crystallography, differential scanning calorimetry (DSC), dynamic vapor sorption/desorption (DVS), Karl Fischer analysis (KF), hot stage microscopy, modulated differential screening calorimetry, FT-IR, and Raman spectroscopy. In particular, analysis of the XRPD and DSC data can distinguish Form A, Form B, and a hemibenzenate form of CDDO-Me. See U.S. Patent Application Publication 2009/0048204, which is incorporated by reference herein in its entirety.

Additional details regarding polymorphic forms of CDDO-Me are described in U.S. Patent Application Publication 2009/0048204, PCT Publication WO 2009/023232 and PCT Publication WO 2010/093944, which are all incorporated herein by reference in their entireties.

Non-limiting specific formulations of the compounds disclosed herein include CDDO-Me polymer dispersions. See, for example, PCT Publication WO 2010/093944, which is incorporated herein by reference in its entirety. Some of the formulations reported therein exhibit higher bioavailability than either the micronized Form A or nanocrystalline Form A formulations. Additionally, the polymer dispersion-based formulations demonstrate further surprising improvements in oral bioavailability relative to the micronized Form B formulations. For example, the methacrylic acid copolymer, Type C and HPMC-P formulations showed the greatest bioavailability in the subject monkeys.

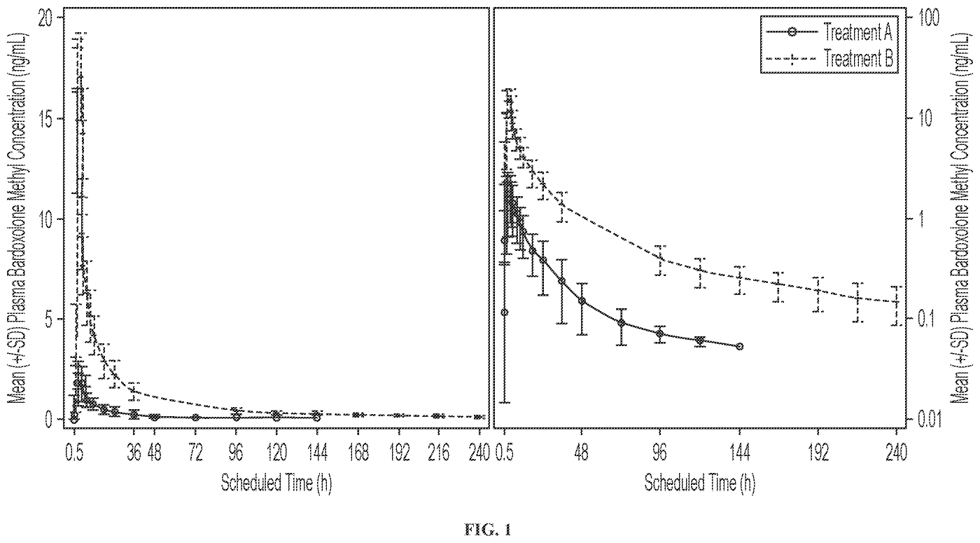

In some embodiments, at least a portion of the CDDO-Me is present as a polymorphic form, wherein the polymorphic form is crystalline form having an X-ray diffraction pattern (CuKα) comprising significant diffraction peaks at about 6.2, 12.4, 15.4, 18.6 and 24.9° 2θ. In some aspects, the crystalline form is further characterized by one, two, three, four or five additional diffraction peaks selected from the group consisting of 8.6, 13.3, 13.7, 17.1 and 21.7° 2θ. In non-limited examples, the X-ray diffraction pattern (CuKα) is substantially as shown in FIG. 1 of WO 2019/014412, which is incorporated herein by reference in its entirety.

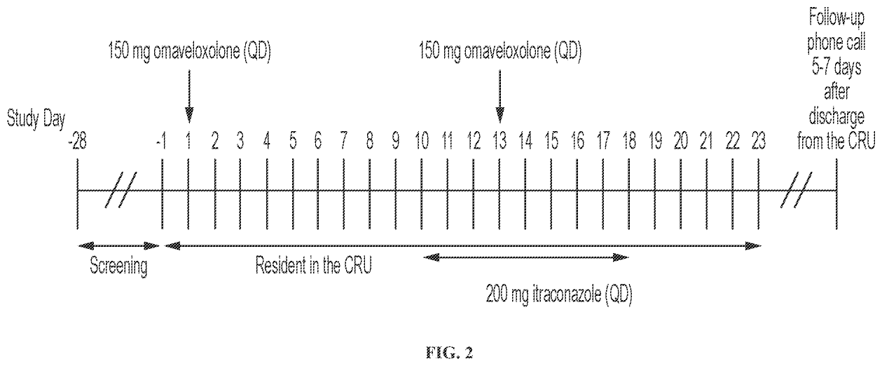

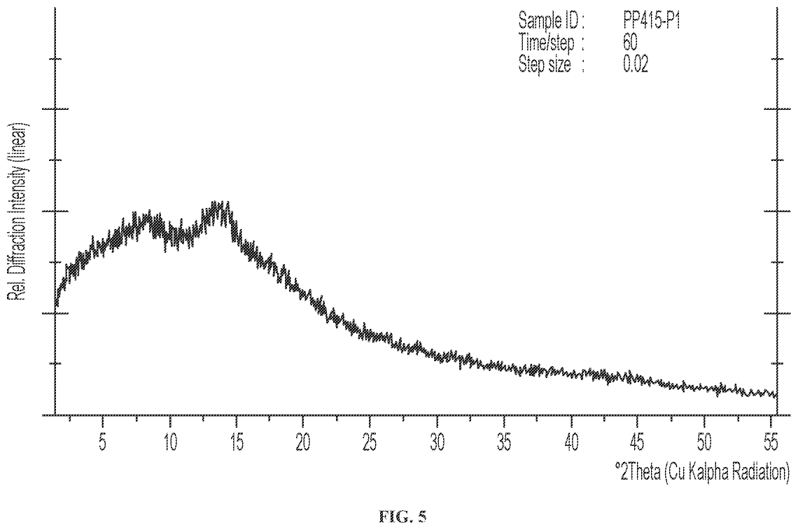

In some embodiments, at least a portion of the CDDO-Me is present as a polymorphic form, wherein the polymorphic form is crystalline form having an X-ray diffraction pattern (CuKα) comprising significant diffraction peaks at about 3.6, 7.1, 10.8, 12.4 and 16.5° 2θ. In some aspects, the crystalline form is further characterized by one, two, three, four or five additional diffraction peaks selected from the group consisting of 12.9, 13.9, 14.8, 18.6 and 20.6° 2θ. In non-limited examples, the X-ray diffraction pattern (CuKα) is substantially as shown in FIG. 2 of WO 2019/014412, which is incorporated herein by reference in its entirety. In some aspects, the crystalline form is further characterized by a Raman spectrum having peaks at 2949, 1671, 1618 and 1464±4 cm⁻¹. In non-limited examples, the Raman spectrum is substantially as shown in FIGS. 4 and 5 of WO 2019/014412, which is incorporated herein by reference in its entirety.

In some embodiments, at least a portion of the CDDO-Me is present as a polymorphic form, wherein the polymorphic form is a toluene solvate crystalline form having an X-ray diffraction pattern (CuKα) comprising diffraction peaks at about 9.65, 7.58, 7.18, 6.29, 6.06, 5.47, 5.21, 4.77 and 3.07° 2θ. In non-limited examples, the X-ray diffraction pattern (CuKα) is substantially as shown in FIG. 1 of CN102887936, which is incorporated herein by reference in its entirety.

In some embodiments, at least a portion of the CDDO-Me is present as a polymorphic form, wherein the polymorphic form is a semi-dioxane solvate crystalline form having an X-ray diffraction pattern (CuKα) comprising diffraction peaks at about 10.01, 7.09, 6.84, 6.23, 5.29, 5.20, 5.10, 4.84, and 4.61° 2θ. In non-limited examples, the X-ray diffraction pattern (CuKα) is substantially as shown in FIG. 4 of CN102887936, which is incorporated herein by reference in its entirety.

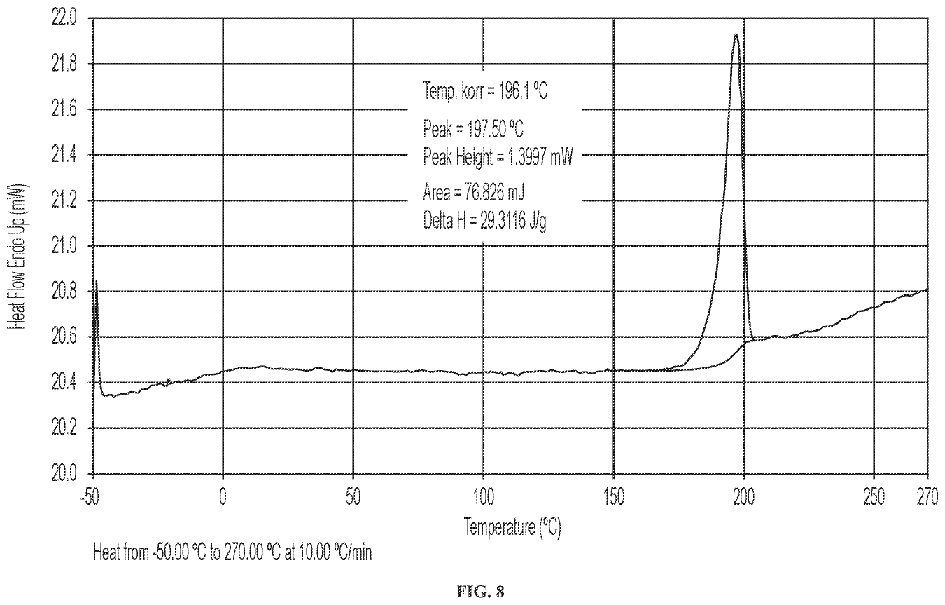

In some embodiments, at least a portion of the CDDO-Me is present as a polymorphic form, wherein the polymorphic form is a semi-tetrahydrofuran solvate crystalline form having an X-ray diffraction pattern (CuKα) comprising diffraction peaks at about 10.00, 7.14, 6.80, 6.65, 6.10, 5.62, 5.29, 4.88, and 4.50° 2θ. In non-limited examples, the X-ray diffraction pattern (CuKα) is substantially as shown in FIG. 8 of CN102887936, which is incorporated herein by reference in its entirety.

In some embodiments, at least a portion of the CDDO-Me is present as a polymorphic form, wherein the polymorphic form is a methanol solvate crystalline form having an X-ray diffraction pattern (CuKα) comprising diffraction peaks at about 8.86, 8.45, 8.17, 7.90, 7.26, 4.67, 6.63, 6.46, and 3.64° 2θ. In non-limited examples, the X-ray diffraction pattern (CuKα) is substantially as shown in FIG. 1 of CN102875634, which is incorporated herein by reference in its entirety.

In some embodiments, at least a portion of the CDDO-Me is present as a polymorphic form, wherein the polymorphic form is an anhydrous crystalline form having an X-ray diffraction pattern (CuKα) comprising diffraction peaks at about 12.05, 8.90, 8.49, 8.13, 7.92, 7.29, 6.64, 4.67 and 3.65° 2θ. In non-limited examples, the X-ray diffraction pattern (CuKα) is substantially as shown in FIG. 2 of CN102875634, which is incorporated herein by reference in its entirety.

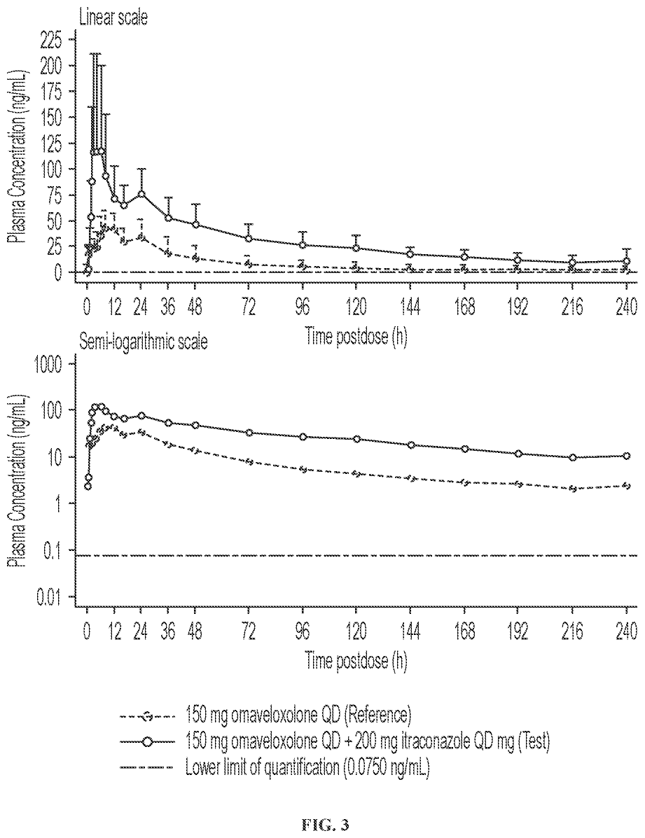

In some embodiments, at least a portion of the CDDO-Me is present as a polymorphic form, wherein the polymorphic form is a dihydrate crystalline form having an X-ray diffraction pattern (CuKα) comprising diffraction peaks at about 8.81, 8.48, 7.91, 7.32, 5.09, 4.24, 3.58, 3.36 and 3.17° 2θ. In non-limited examples, the X-ray diffraction pattern (CuKα) is substantially as shown in FIG. 3 of CN102875634, which is incorporated herein by reference in its entirety.

Prodrug forms of bardoxolone methyl may be used in accordance with the methods of this invention. Since prodrugs are known to enhance numerous desirable qualities of pharmaceuticals, e.g., solubility, bioavailability, manufacturing, etc., the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively. In some embodiments, the α-cyano-substituted α,β-unsaturated ketone in the A ring of bardoxolone methyl can be converted to an enol, which can be esterified or etherified to generate prodrugs. For example, CDDO-AZO may be used with the methods provided herein. See Qiao et al., Chinese Journal of Natural Medicines, 19:545-550, 2021, which is incorporated by reference herein in its entirety. CDDO-AZO has the structural formula:

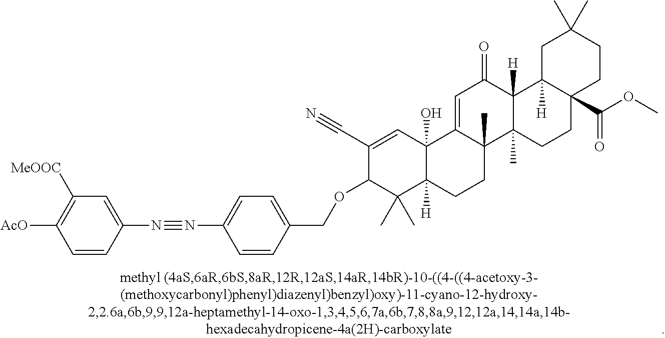

### B. Omaveloxolone

Omaveloxolone (RTA 408; 63415) can be prepared according to the methods described in the Examples section below. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

RTA 408 may also exist in prodrug form. Since prodrugs are known to enhance numerous desirable qualities of pharmaceuticals, e.g., solubility, bioavailability, manufacturing, etc., the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

RTA 408 may contain one or more asymmetrically substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. RTA 408 may occur as racemates and racemic mixtures, single enantiomers, diastereomeric mixtures and individual diastereomers. In some embodiments, a single diastereomer is obtained. The chiral centers of RTA 408 according to the present invention can have the S or the R configuration.

In addition, atoms making up RTA 408 of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of RTA 408 may be replaced by a sulfur or selenium atom(s).

RTA 408 and polymorphic form thereof may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical advantages over, compounds known in the prior art for use in the indications stated herein.

### 1. RTA 408 Amorphous Form

In some embodiments, different solid forms of RTA 408 may be used. RTA 408 has a high tendency for solvate formation. Crystalline forms of classes 2, 3, 4, and 5 are consistent with solvates. For a description of the classes, see Table 1 below. Attempts to dry classes 2 and 3 (two groups of isostructural solvates) were not successful, which is consistent with tightly bound solvent molecules. In some embodiments, drying of a class 4 solid (acetonitrile solvate) led to an isostructural desolvated form. In some embodiments, drying of a class 5 solid (THF solvate) resulted in the amorphous form class 1. Non-solvated forms of RTA 408 include the amorphous form (class 1) and the crystalline desolvated solvate of class 4 (isostructural to the class 4 acetonitrile solvate). In some embodiments, the amorphous form has a high glass transition with T_{g}≈153° C. (ΔCp=0.72 J/g° C.) and is only slightly hygroscopic (Δm=+0.4% 50%→85% r.h.). In some embodiments, RTA 408 may be present as an amorphous form is stable for at least four weeks under elevated temperature and humidity conditions (i.e., open at 40° C./˜75% r.h. or closed at 80° C.). In some embodiments, the amorphous form (class 1) was successfully prepared from class 2 material in a two-step process (transformation into class 5 and subsequent drying of class 5 to obtain the amorphous form), as well as in a direct one-step method (precipitation from an acetone solution in a cold water bath). The crystalline desolvated solvate of class 4 (isostructural to the class 4 solvate) is slightly hygroscopic (mass gain of ˜0.7 wt.-% from 50% r.h. to 85% r.h.) and has a possible melting point at 196.1° C. (ΔH=29.31 J/g).

A sample of the amorphous form of 63415, class 1, was characterized by FT-Raman spectroscopy, PXRD, TG-FTIR, Karl Fischer titration, ¹H-NMR, DSC, and DVS (see Examples section for additional details). The sample was found to contain ˜0.9 wt.-% EtOH with traces of H₂O (according to the TG-FTIR). A water content of 0.5 wt.-% was determined by Karl Fischer titration. DSC shows a high glass transition temperature with T_{g}≈153° C. (ΔCp=0.72 J/g° C.). According to DVS, the material is slightly hygroscopic (Δm=+0.4% 50%→85% r.h.). No crystallization was observed in the DSC or DVS experiments.

The chemical stability of the amorphous form was investigated in organic solvents, including acetone, EtOAc, MeOH, and MeCN, as well as different aqueous media (e.g., 1% aq. Tween 80, 1% aq. SDS, 1% aq. CTAB) at a concentration of 1 mg/mL at time points 6 h, 24 h, 2 d, and 7 d. Decomposition ≥1% was observed only for solutions in MeCN after 7 days and for suspensions in the 1% aqueous Tween 80 medium (at all times points at 254 nm and after 24 h, 2 d, and 7 d at 242 nm).

In addition, the stability of the amorphous form was investigated by storage under elevated temperature and humidity conditions (open at 25° C./62% r.h. and 40° C./75% r.h. and closed at 60° C. and 80° C.). After one week, two weeks, and four weeks, the stored samples were analyzed by PXRD. None of the samples differed from the amorphous starting material.

More than 30 crystallization and drying experiments were carried out, including suspension equilibration, slow cooling, evaporation, and precipitation. Four new crystalline forms were obtained (classes 2, 3, 4, and 5) in addition to the amorphous form (class 1).

The four new forms (classes 2, 3, 4, and 5) were characterized by FT-Raman spectroscopy, PXRD, and TG-FTIR. All forms correspond to solvates (Table 1). Drying experiments under vacuum or N₂ flow were carried out with the aim to obtain a crystalline, non-solvated form of 63415.

TABLE 1
Summary of Obtained Classes

| | | Result of Drying |
|---|---|---|
| Class | Characteristics | Experiments |
| Class 1 | amorphous form | — |
| Class 2 | isostructural solvates (e.g., heptane) | drying not successful |
| Class 3 | isostructural solvates (e.g., ethanol) | drying not successful |
| Class 4 | MeCN solvate & desolvated solvate | drying successful, |
| | | structure unchanged |
| Class 5 | THE solvate | drying resulted in |
| | | amorphous form |

Class 2: Most crystallization experiments that were conducted resulted in solid material of class 2. Its members may correspond to isostructural, non-stoichiometric (<0.5 eq.) solvates (of heptane, cyclohexane, isopropyl ether, 1-butanol, triethylamine, and possibly other solvents, such as hexane, other ethers, etc.) with tightly bound solvent molecules. The Raman spectra and PXRD patterns within this class are very similar to each other, thus the structures might be essentially identical with only small differences due to the different solvents that were incorporated.

Drying experiments on class 2 samples have not resulted in a crystalline, non-solvated form. Even elevated temperatures (80° C.) and a high vacuum (<1×10⁻³ mbar) could not remove the tightly bound solvent molecules completely; a solvent content of >2 wt.-% always remained. The crystallinity of these partially dried samples is reduced, but neither transformation into a different structure nor substantial amorphization was observed.

Class 3: Solid material of class 3 may be obtained from several crystallizations. The samples of class 3 are likely isostructural solvates of 2PrOH, EtOH, and probably acetone with tightly bound solvent molecules. They could correspond to either stoichiometric hemisolvates or non-stoichiometric solvates with a solvent content of ˜0.5 eq. As with class 2, the Raman spectra and PXRD patterns within this class are very similar to each other, indicating similar structures that incorporate different solvents.

Similar to class 2, drying experiments were not successful. The very tightly bound solvent molecules could only partially be removed (i.e., ˜5.4 wt.-% to ˜4.8 wt.-% after up to 3 d at 1×10⁻³ mbar and 80° C.). The PXRD patterns remained unchanged.

Class 4 may be obtained from a 7:3 MeCN/H₂O solvent system. It most likely corresponds to a crystalline acetonitrile hemisolvate. By drying (under vacuum or N₂ flow at elevated temperatures) most of the solvent molecules could be removed without changing or destroying the crystal structure (PXRD remained unchanged). Thus, a crystalline, non-solvated form (or rather desolvated solvate) was obtained. It is slightly hygroscopic (mass gain of ˜0.7 wt.-% from 50% r.h. to 85% r.h.) and has a possible melting point at 196.1° C. (ΔH=29.31 J/g).

Class 5 may be obtained from an ˜1:1 THF/H₂O solvent system. Class 5 contains bound THF (and maybe H₂O). As the content of the two components cannot be readily quantified separately, the exact nature of this crystalline solvate has not been determined.

Drying of class 5 resulted in significant desolvation and transformation in the direction of the amorphous form (class 1). In some embodiments, the amorphous form of RTA 408 may be prepared by suspending class 2 heptane solvate in 1:1 THF/H₂O to form a class 5 solid, followed by drying and amorphization.

Experiments with the aim of preparing the amorphous form (class 1) were carried out using class 2 starting material. Mainly amorphous material (class 1) was prepared starting from class 2 material in a two-step process via class 5 on a 100-mg and 3-g scale (drying at 100 mbar, 80° C., several days). The preparation of fully amorphous material (class 1) was found to be possible in a one-step process avoiding the solvent THE by direct precipitation of the amorphous form (class 1) from an acetone solution of class 2 material in a cold water bath.

### 2. RTA 408 Crystalline Polymorphic Form A

Example 1: 17 g of RTA 408 was dissolved in 68 g of acetone. 620 g of de-ionized water was added to a 500 mL jacketed reactor and cooled to 2° C. When the water was below 7° C., the RTA 408 solution was added to the reactor via an addition funnel. A slurry of solids formed. The slurry was stirred in the reactor with nitrogen purge. Solids were isolated using vacuum filtration and dried under vacuum at room temperature to give Form A.

Example 2: 300 mg of RTA 408 was dissolved in 1 mL of ethyl acetate. To the clear solution, 2 mL of heptane was added. Crystallization occurred within 30 minutes. The slurry was stirred overnight and the solids were isolated by vacuum filtration and dried at ambient temperature for 1 hour. The solids were then dried in a vacuum oven at 50° C. overnight to give Form A.

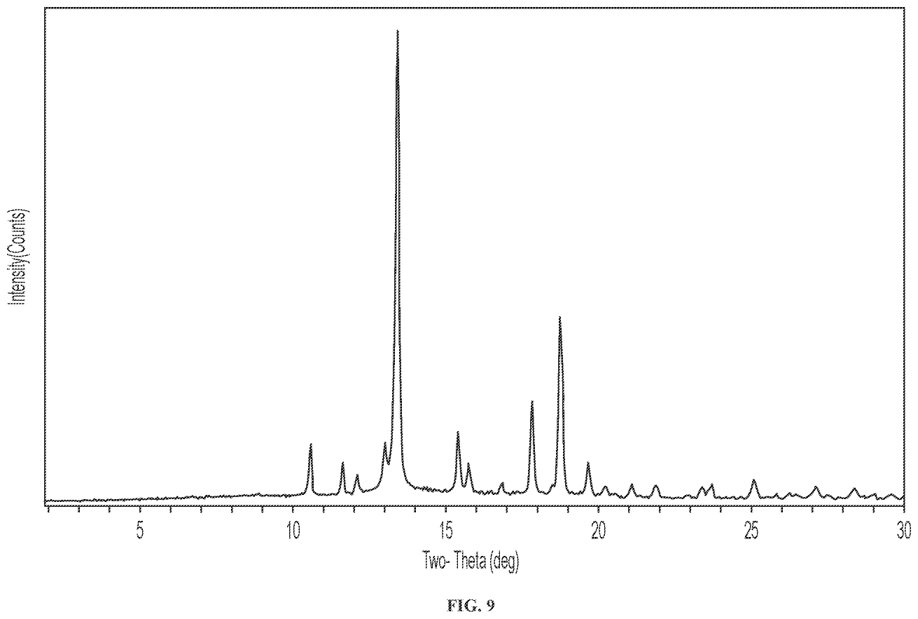

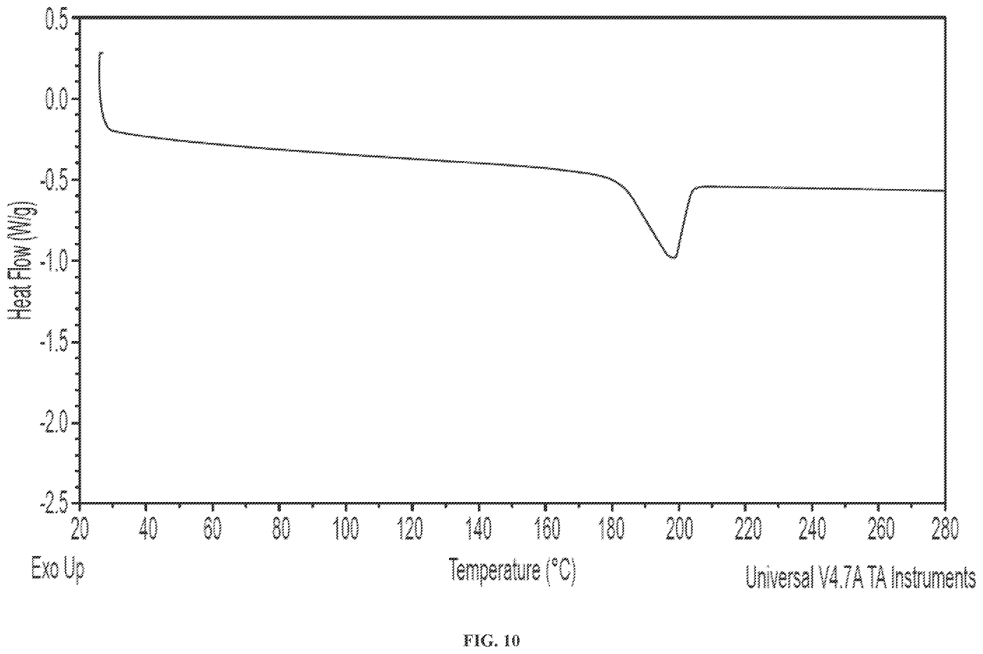

Powder X-ray diffraction (PXRD) pattern and peak listing with relative intensities are shown in FIG. 9 and Table 2, respectively. Differential scanning calorimetry (DSC) is shown in FIG. 10.

The DSC of Form A indicated an essentially solvent free form with a melting point of 181.98° C. and enthalpy of fusion of 42.01 J/g. The TGA-MS of Form A shows the loss of ˜0.5 wt.-% with traces of H₂O between 25 and 200° C., predominantly above 160° C., indicating that RTA 408 Polymorphic Form A may be slightly hygroscopic.

TABLE 2
Peak Listing of RTA 408 Form A.

| Peak Position (°2θ) | Relative Intensity |
|---|---|
| 10.601 | 11.0 |
| 11.638 | 7.1 |
| 12.121 | 4.6 |
| 13.021 | 10.9 |
| 13.435 | 100.0 |
| 15.418 | 12.7 |
| 15.760 | 5.9 |
| 17.830 | 19.7 |
| 18.753 | 38.3 |
| 19.671 | 7.5 |

### 3. RTA 408 Crystalline Polymorphic Form B

Example 3: 1.0 g of RTA 408 was dissolved in 1.5 mL of acetone. In a scintillation vial, 10 mL of de-ionized water was heated to 50° C. and the RTA 408 solution was added to the vial dropwise. Upon stirring for 2 hours, a slurry of solids formed. The slurry was then cooled to room temperature. The resulting solids were isolated by filtration and dried in a vacuum oven at 50° C. overnight to give Form B.

Example 4: 2.9 g of RTA 408 was dissolved in 20 mL of isopropyl alcohol at reflux. 20 mL of heptane was added to the solution at reflux. The solution was cooled to room temperature and mixed for 1 hour. A slurry of solids formed. The solids were isolated by vacuum filtration and dried under vacuum at ambient temperature to give Form B.

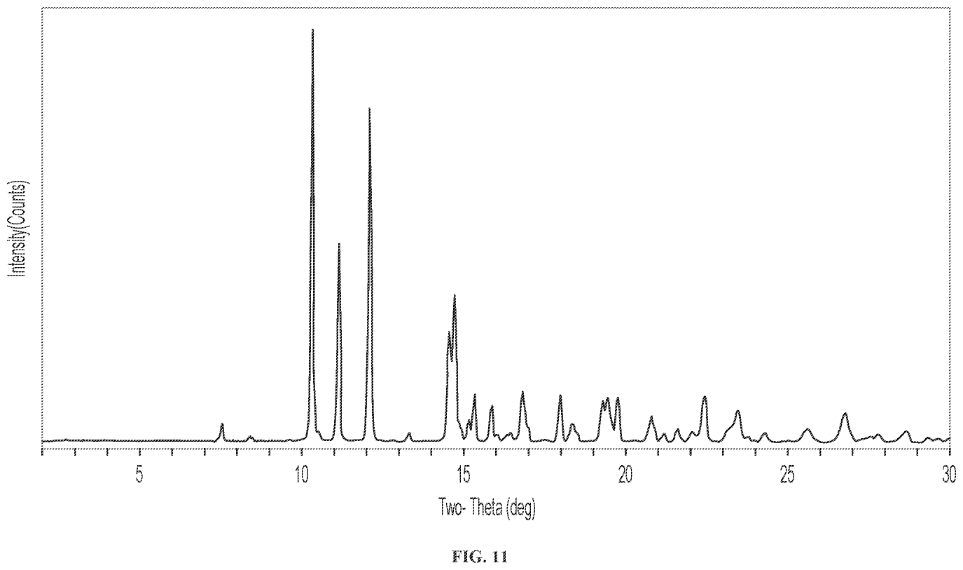

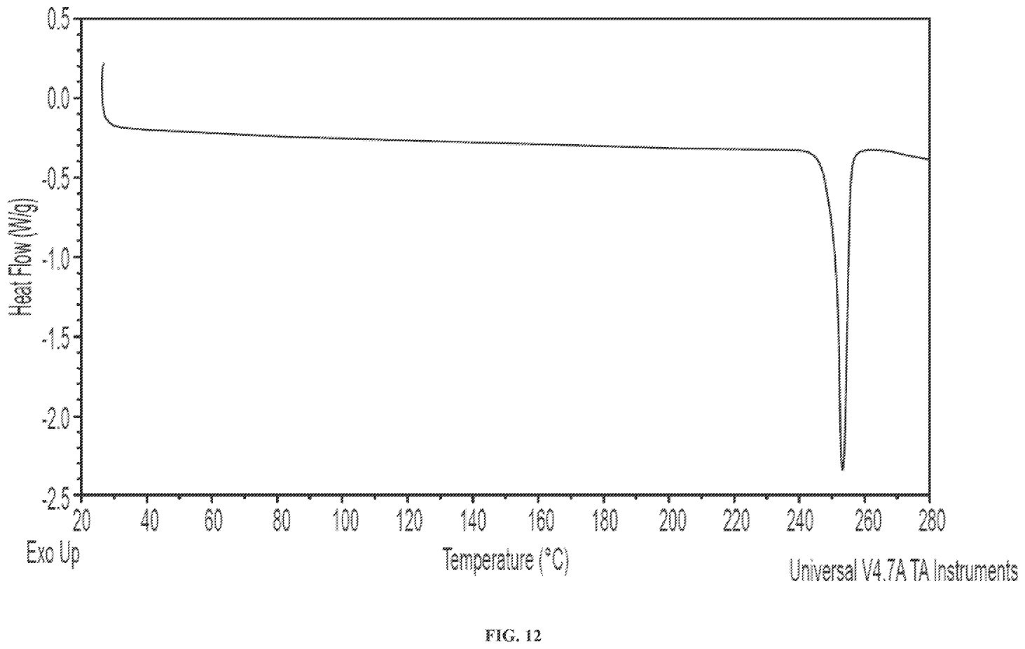

Powder X-ray diffraction (PXRD) pattern and peak listing with relative intensities are shown in FIG. 11 and Table 3, respectively. Differential scanning calorimetry (DSC) is shown in FIG. 12.

The DSC of Form B indicated an essentially solvent free form with a melting point of 250.10° C. and enthalpy of fusion of 42.01 J/g. The TGA-MS of Form B shows the slight loss of ˜0.2 wt.-% with traces of H₂O between 25 and 200° C., indicating that RTA 408 Polymorphic Form B may be very slightly hygroscopic.

TABLE 3
Peak Listing of RTA 408 Form B

| Peak Position (°2θ) | Relative Intensity |
|---|---|
| 7.552 | 4.2 |
| 10.339 | 100.0 |
| 11.159 | 48.4 |
| 12.107 | 80.7 |
| 14.729 | 35.2 |
| 15.329 | 11.4 |
| 15.857 | 8.4 |
| 16.824 | 11.3 |
| 17.994 | 10.9 |
| 18.344 | 4.1 |
| 19.444 | 10.4 |
| 19.764 | 10.2 |
| 20.801 | 5.7 |
| 22.414 | 10.1 |

### C. Exemplary Measurement Methods

### 1. Powder X-Ray Diffractometry (PXRD)

PXRD data were collected using a G3000 diffractometer (Inel Corp., Artenay, France) equipped with a curved position sensitive detector and parallel beam optics. The diffractometer was operated with a copper anode tube (1.5 kW fine focus) at 40 kV and 30 mA. An incident beam germanium monochromometer provided monochromatic radiation. The diffractometer was calibrated using the attenuated direct beam at one-degree intervals. Calibration was checked using a silicon powder line position reference standard (NIST 640c). The instrument was computer controlled using the Symphonix software (Inel Corp., Artenay, France) and the data was analyzed using the Jade software (version 9.0.4, Materials Data, Inc., Livermore, CA). The sample was loaded onto an aluminum sample holder and leveled with a glass slide.

### 2. Thermo Gravimetric Analysis/Mass Spectrometry

The TGA was run with TA instruments, data were collected on a thermal balance (Q-5000, TA Instruments, New Castle, DE) equipped with a data analyzer (Universal Analysis 2000, version 4.5A, TA Instruments, New Castle, DE). During experiments, the furnace was purged with nitrogen at 60 mL/minute, while the balance chamber was purged at 40 mL/minute. Temperature of the TGA furnace was calibrated using curie points of aluminum and nickel. Sample size ranged from 2 to 20 mg, and a heating rate of 10° C./minute was used.

For TGA-MS, the thermogravimetric analysis part was the same as above. The mass of evolved gas was analyzed with PFEIFFER GSD 301 T3 ThermoStar (PFEIFFER Vacuum, Asslar, Germany). The instrument was operated and data evaluated with Software Quadstar 32-bit (V7.01, Inficon, LI-9496 Balzers, Liechtenstein).

### 3. Differential Scanning Calorimetery

A DSC (Q-2000, TA Instruments, New Castle, DE) equipped with Universal Analysis 2000 software (Version 4.5A, TA Instruments, New Castle, DE) was used to determine the DSC thermal traces. The temperature axis was calibrated with biphenyl, indium, and tin standards. The cell constant was calibrated with indium. Unless otherwise stated, the sample (2-5 mg) was encapsulated in a ventilated aluminum pan, and heated at a rate of 10° C./minute under a nitrogen gas flow of 50 mL/minute during the study.

### II. CYP3A4 Modulators

CYP450 inhibition means that one drug (inhibitor) decreases the activity of an enzyme and consequently increases the blood concentration of another drug, the substrate of the enzyme. A substance is an “inhibitor” of CYP3A4 activity when the specific activity of the enzyme is decreased by the presence of the substance, without reference to the precise mechanism of such decrease. For example, a substance can be an inhibitor of enzyme activity by competitive, non-competitive, allosteric or other type of enzyme inhibition, by decreasing expression of the enzyme, or other direct or indirect mechanisms. Co-administration of a given drug with an inhibitor may decrease the rate of metabolism of that drug through the metabolic pathway listed. By way of example, strong inhibitors of CYP3A4 include clarithromycin, indinavir, nefazodone, saquinavir, suboxone, telithromycin, erythromycin, diltiazem, itraconazole, ketoconazole, ritonavir, and goldenseal. Intermediate strength inhibitors include aprepitant, erythromycin, fluconazole, grapefruit, verapamil, and diltiazem. Weak inhibitors include cimetidine. Other possible inhibitors include amiodarone, boceprevir, chloramphenicol, ciprofloxacin, delaviridine, diethyl-dithiocarbamate, fluvoxamine, gestodene, imatinib, mibefradil, mifepristone, norfloxacin, norfluoxetine, starfruit, telaprevir, and voriconazole.

On the other hand, CYP450 induction increases the capacity of CYP450 to metabolize drugs, thereby reducing blood levels. A substance is an “inducer” of CYP3A4 when the specific activity of the enzyme can be increased by the presence of the substance, without reference to the precise mechanism of such increase. For example, a substance can be an inducer of enzyme activity by increasing reaction rate, by increasing expression of the enzyme, by allosteric activation or other direct or indirect mechanisms. Co-administration of a given drug with an enzyme inducer may increase the rate of excretion of the drug metabolized through the pathway indicated. CYP450 induction also occurs when hepatic blood flow increases or the production of CYP450 increases due to certain drugs or environmental pollutants. By way of example, inducers of CYP3A4 include barbiturates, carbamazepine, efavirenz, modafinil, nevirapine, oxcarbazepine, pioglitazone, rifabutin, troglitazone, phenobarbital, phenytoin, rifampin, St. John's Wort and glucocorticoids.

CYP3A4 modulators can be identified by monitoring the transcriptional responsiveness of the gene and by measuring enzymatic activity towards model substrates (i.e. testosterone). For example, transcriptional responsiveness to prototypical pharmacological CYP3A4 inducers (i.e. rifampin) can be assayed by the reverse transcription polymerase chain reaction (RT-PCR) using specific primers to detect CYP3A4 mRNA. Rifampin-induced CYP3A4 enzymatic activity can also be measured by the production of the 60-OH-testosterone metabolite when cells are incubated with testosterone.

### III. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof, in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of this disclosure, the formulas:

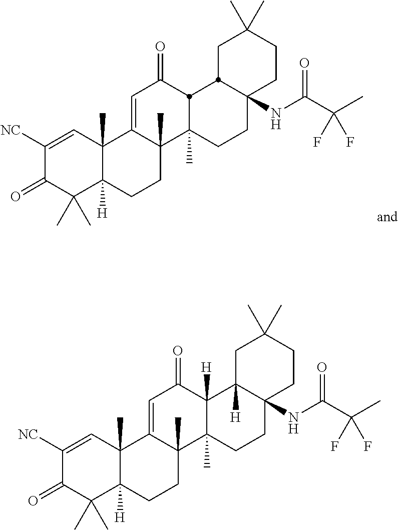

represent the same structures. When a dot is drawn on a carbon, the dot indicates that the hydrogen atom attached to that carbon is coming out of the plane of the page.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

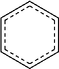

covers, for example,

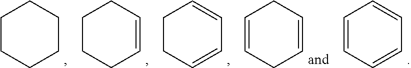

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g., [symbol] for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

A bold dot on a carbon atom indicates that the hydrogen attached to that carbon is oriented out of the plane of the paper. For example, the following two depictions are equivalent:

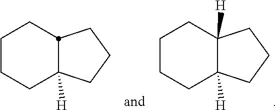 [SMILES: C1CC[C@@H]2CCC[C@H]2C1 ; C1CC[C@@H]2CCC[C@H]2C1]

When a variable is depicted as a “floating group” on a ring system, for example, the group “R” in the formula:

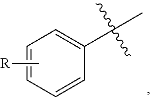

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

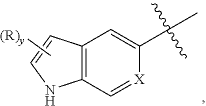

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

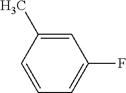

is also taken to refer to

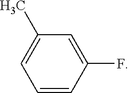

Aromatic compounds may also be depicted using a circle to represent the delocalized nature of the electrons in the fully conjugated cyclic π system, two non-limiting examples of which are shown below:

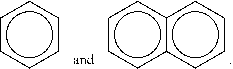

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

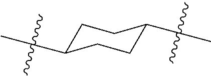 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

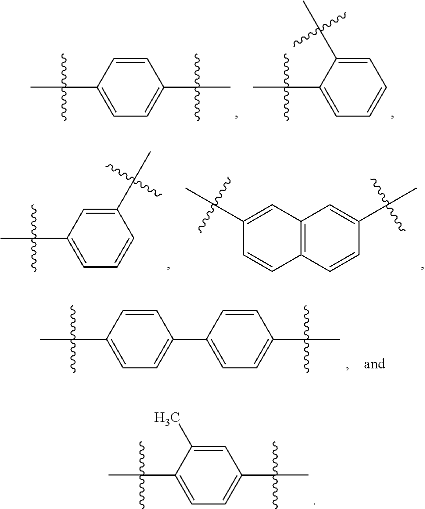

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused or spirocyclic. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. Non-limiting examples of N-heterocycloalkyl groups include N-pyrrolidinyl and

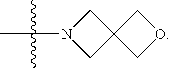 [SMILES: *N1CC2(COC2)C1]

When the term “heterocycloalkyl” is used with the “substituted” modifier, one or more hydrogen atom has been replaced, independently at each instance, by oxo, —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted heterocycloalkyl groups (more specifically, substituted N-heterocycloalkyl groups):

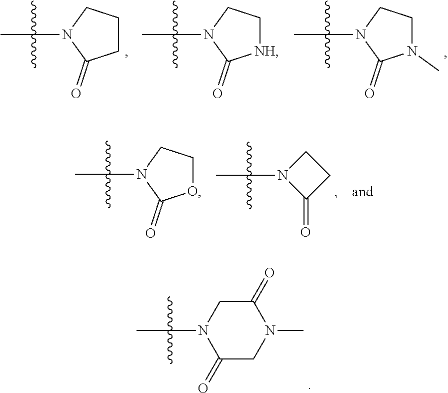

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The terms “cycloalkylamino” and “heterocycloalkylamino”, when used without the “substituted” modifier, refers to groups, defined as —NHR, in which R is cycloalkyl and heterocycloalkyl, respectively. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

The terms “alkylsulfonyl” and “alkylsulfinyl” when used without the “substituted” modifier refers to the groups —S(O)₂R and —S(O)R, respectively, in which R is an alkyl, as that term is defined above. The terms “cycloalkylsulfonyl”, “alkenylsulfonyl”, “alkynylsulfonyl”, “arylsulfonyl”, “aralkylsulfonyl”, “heteroarylsulfonyl”, and “heterocycloalkylsulfonyl” are defined in an analogous manner. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

With the exception of the term “heterocycloalkyl”, when a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂C1 is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

Some of the abbreviations used herein are as follows: Ac indicates an acetyl group (—C(O)CH₃) Boc refers to tert-butyloxycarbonyl; COX-2, cyclooxygenase-2; cyPGs refers to cyclopentenone prostaglandins; DBDMH refers to 1,3-Dibromo-5,5-dimethylhydantoin; DIBAL-H is diisobutylaluminium hydride; DMAP refers to 4-dimethylaminopyridine; DMF is dimethylformamide; DMSO is dimethyl sulfoxide; EDC refers to 1-ethyl-3-(3-dimethylaminopropyl)carbodiimide; Et₂O, diethyl ether; HO-1 stands for inducible heme oxygenase IFNγ or IFN-γ stand for interferon-γ; IL-10 stands for interleukin-1β; iNOS stands for inducible nitric oxide synthase; NCS refers to N-Chlorosuccinimide; NMO refers to N-methylmorpholine N-oxide; NO stands for nitric oxide; Py stands for Pyridine; T3P refers to propylphosphonic anhydride; TFA is trifluoroacetic acid; THE is tetrahydrofuran; TNFα or TNF-α, tumor necrosis factor-α; TPAP is tetrapropylammonium perruthenate; Ts stands for tosyl; TsOH or p-TsOH is p-toluenesulfonic acid.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, the variation that exists among the study subjects, or a value that is within 10% of a stated value. When used in the context of X-ray powder diffraction, the term “about” is used to indicate a value of 0.2° 2θ from the reported value, preferably a value of ±0.1° 2θ from the reported value. When used in the context of differential scanning calorimetry or glass transition temperatures, the term “about” is used to indicate a value of ±10° C. relative to the maximum of the peak, preferably a value of ±2° C. relative to the maximum of the peak. When used in other contexts, the term “about” is used to indicate a value of 10% of the reported value, preferably a value of ±5% of the reported value. It is to be understood that, whenever the term “about” is used, a specific reference to the exact numerical value indicated is also included.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

As used herein, average molecular weight refers to the weight average molecular weight (Mw) as determined by static light scattering.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

The term “halo peak” in the context of X-ray powder diffraction would mean a broad peak, often spanning >10° 2θ in an X-ray powder diffractogram, typically characteristic of an amorphous solid or system.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose that is 50% of the maximum response obtained. This quantitative measure indicates how much of a particular drug or other substance (inhibitor) is needed to inhibit a given biological, biochemical, or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a non-human animal. In certain embodiments, the patient or subject is a primate. In certain embodiments, the patient or subject is a human. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical agent, pharmaceutical preparation, pharmaceutical composition, pharmaceutical formulation, pharmaceutical product, medicinal product, medicine, medication, medicament, or simply a drug) is a drug used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-p-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### IV. Diseases Associated with Inflammation and/or Oxidative Stress

Inflammation is a biological process that provides resistance to infectious or parasitic organisms and the repair of damaged tissue. Inflammation is commonly characterized by localized vasodilation, redness, swelling, and pain, the recruitment of leukocytes to the site of infection or injury, production of inflammatory cytokines, such as TNF-α and IL-1, and production of reactive oxygen or nitrogen species, such as hydrogen peroxide, superoxide, and peroxynitrite. In later stages of inflammation, tissue remodeling, angiogenesis, and scar formation (fibrosis) may occur as part of the wound healing process. Under normal circumstances, the inflammatory response is regulated, temporary, and is resolved in an orchestrated fashion once the infection or injury has been dealt with adequately. However, acute inflammation can become excessive and life-threatening if regulatory mechanisms fail. Alternatively, inflammation can become chronic and cause cumulative tissue damage or systemic complications. The synthetic triterpenoid compounds of this disclosure can be used in the treatment or prevention of inflammation or diseases associated with inflammation.

Many serious and intractable human diseases involve dysregulation of inflammatory processes, including diseases such as cancer, atherosclerosis, and diabetes, which were not traditionally viewed as inflammatory conditions. In the case of cancer, the inflammatory processes are associated with processes that include tumor formation, progression, metastasis, and resistance to therapy. In some embodiments, the synthetic triterpenoid compounds of this disclosure may be used in the treatment or prevention of cancers including a carcinoma, sarcoma, lymphoma, leukemia, melanoma, mesothelioma, multiple myeloma, or seminoma, or cancer of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

Atherosclerosis, long viewed as a disorder of lipid metabolism, is now understood to be primarily an inflammatory condition, with activated macrophages playing an important role in the formation and eventual rupture of atherosclerotic plaques. Activation of inflammatory signaling pathways has also been shown to play a role in the development of insulin resistance, as well as in the peripheral tissue damage associated with diabetic hyperglycemia. Excessive production of reactive oxygen species and reactive nitrogen species, such as superoxide, hydrogen peroxide, nitric oxide, and peroxynitrite, is a hallmark of inflammatory conditions. Evidence of dysregulated peroxynitrite production has been reported in a wide variety of diseases (Szabo et al., 2007; Schulz et al., 2008; Forstermann, 2006; Pall, 2007).

Autoimmune diseases such as rheumatoid arthritis, lupus, psoriasis, and multiple sclerosis involve inappropriate and chronic activation of inflammatory processes in affected tissues, arising from dysfunction of self vs. non-self recognition and response mechanisms in the immune system. In neurodegenerative diseases such as Alzheimer's and Parkinson's diseases, neural damage is correlated with activation of microglia and elevated levels of pro-inflammatory proteins, such as inducible nitric oxide synthase (iNOS). Chronic organ failure, such as renal failure, heart failure, liver failure, and chronic obstructive pulmonary disease, is closely associated with the presence of chronic oxidative stress and inflammation, leading to the development of fibrosis and eventual loss of organ function. Oxidative stress in vascular endothelial cells, which line major and minor blood vessels, can lead to endothelial dysfunction and is believed to be an important contributing factor in the development of systemic cardiovascular disease, complications of diabetes, chronic kidney disease and other forms of organ failure, and a number of other aging-related diseases, including degenerative diseases of the central nervous system and the retina.

Many other disorders involve oxidative stress and inflammation in affected tissues, including inflammatory bowel disease; inflammatory skin diseases; mucositis and dermatitis related to radiation therapy and chemotherapy; eye diseases, such as uveitis, glaucoma, macular degeneration, and various forms of retinopathy; transplant failure and rejection; ischemia-reperfusion injury; chronic pain; degenerative conditions of the bones and joints, including osteoarthritis and osteoporosis; asthma and cystic fibrosis; seizure disorders; and neuropsychiatric conditions, including schizophrenia, depression, bipolar disorder, post-traumatic stress disorder, attention deficit disorders, autism-spectrum disorders, and eating disorders, such as anorexia nervosa. Dysregulation of inflammatory signaling pathways is believed to be a major factor in the pathology of muscle wasting diseases, including muscular dystrophy and various forms of cachexia.

A variety of life-threatening acute disorders also involve dysregulated inflammatory signaling, including acute organ failure involving the pancreas, kidneys, liver, or lungs, myocardial infarction or acute coronary syndrome, stroke, septic shock, trauma, severe burns, and anaphylaxis.

Many complications of infectious diseases also involve dysregulation of inflammatory responses. Although an inflammatory response can kill invading pathogens, an excessive inflammatory response can also be quite destructive and in some cases can be a primary source of damage in infected tissues. Furthermore, an excessive inflammatory response can also lead to systemic complications due to overproduction of inflammatory cytokines, such as TNF-α and IL-1. This is believed to be a factor in mortality arising from severe influenza, severe acute respiratory syndrome, and sepsis.

The aberrant or excessive expression of either iNOS or cyclooxygenase-2 (COX-2) has been implicated in the pathogenesis of many disease processes. For example, it is clear that NO is a potent mutagen (Tamir and Tannebaum, 1996), and that nitric oxide can also activate COX-2 (Salvemini et al., 1994). Furthermore, there is a marked increase in iNOS in rat colon tumors induced by the carcinogen, azoxymethane (Takahashi et al., 1997). A series of synthetic triterpenoid analogs of oleanolic acid have been shown to be powerful inhibitors of cellular inflammatory processes, such as the induction by IFN-γ of inducible nitric oxide synthase (iNOS) and of COX-2 in mouse macrophages. See Honda et al. (2000a), Honda et al. (2000b), and Honda et al. (2002), which are all incorporated herein by reference.

In one aspect, the synthetic triterpenoid compounds of this disclosure are in part characterized by their ability to inhibit the production of nitric oxide in macrophage-derived RAW 264.7 cells induced by exposure to y-interferon. The synthetic triterpenoid compounds of this disclosure are further characterized by their ability to induce the expression of antioxidant proteins, such as NQO1, and reduce the expression of pro-inflammatory proteins, such as COX-2 and inducible nitric oxide synthase (iNOS). These properties are relevant to the treatment of a wide array of diseases and disorders involving oxidative stress and dysregulation of inflammatory processes, including cancer, complications from localized or total-body exposure to ionizing radiation, mucositis and dermatitis resulting from radiation therapy or chemotherapy, autoimmune diseases, cardiovascular diseases, including atherosclerosis, ischemia-reperfusion injury, acute and chronic organ failure, including renal failure and heart failure, respiratory diseases, diabetes and complications of diabetes, severe allergies, transplant rejection, graft-versus-host disease, neurodegenerative diseases, diseases of the eye and retina, acute and chronic pain, degenerative bone diseases, including osteoarthritis and osteoporosis, inflammatory bowel diseases, dermatitis and other skin diseases, sepsis, burns, seizure disorders, and neuropsychiatric disorders.

In another aspect, the synthetic triterpenoid compounds of this disclosure may be used for treating a subject having a condition such as eye diseases. For example, uveitis, macular degeneration (both the dry form and wet form), glaucoma, diabetic macular edema, blepharitis, diabetic retinopathy, diseases and disorders of the corneal endothelium such as Fuchs endothelial corneal dystrophy, post-surgical inflammation, dry eye, allergic conjunctivitis and other forms of conjunctivitis are non-limiting examples of eye diseases that could be treated with the synthetic triterpenoid compounds of this disclosure.

In another aspect, the synthetic triterpenoid compounds of this disclosure may be used for treating a subject having a condition such as skin diseases or disorders. For example, dermatitis, including allergic dermatitis, atopic dermatitis, dermatitis due to chemical exposure, and radiation-induced dermatitis; thermal or chemical burns; chronic wounds including diabetic ulcers, pressure sores, and venous ulcers; acne; alopecia including baldness and drug-induced alopecia; other disorders of the hair follicle; epidermolysis bullosa; sunburn and its complications; disorders of skin pigmentation including vitiligo; aging-related skin conditions; post-surgical wound healing; prevention or reduction of scarring from skin injury, surgery, or burns; psoriasis; dermatological manifestations of autoimmune diseases or graft-versus host disease; prevention or treatment of skin cancer; disorders involving hyperproliferation of skin cells such as hyperkeratosis is a non-limiting example of skin diseases that could be treated with the synthetic triterpenoid compounds of this disclosure.

Without being bound by theory, the activation of the antioxidant/anti-inflammatory Keap1/Nrf2/ARE pathway is believed to be implicated in both the anti-inflammatory and anti-carcinogenic properties of the compound disclosed herein.

In another aspect, the synthetic triterpenoid compounds of this disclosure may be used for treating a subject having a condition caused by elevated levels of oxidative stress in one or more tissues. Oxidative stress results from abnormally high or prolonged levels of reactive oxygen species, such as superoxide, hydrogen peroxide, nitric oxide, and peroxynitrite (formed by the reaction of nitric oxide and superoxide). The oxidative stress may be accompanied by either acute or chronic inflammation. The oxidative stress may be caused by mitochondrial dysfunction, by activation of immune cells, such as macrophages and neutrophils, by acute exposure to an external agent, such as ionizing radiation or a cytotoxic chemotherapeutic agent (e.g., doxorubicin), by trauma or other acute tissue injury, by ischemia/reperfusion, by poor circulation or anemia, by localized or systemic hypoxia or hyperoxia, by elevated levels of inflammatory cytokines and other inflammation-related proteins, and/or by other abnormal physiological states, such as hyperglycemia or hypoglycemia.

In animal models of many such conditions, stimulating expression of inducible heme oxygenase (HO-1), a target gene of the Nrf2 pathway, has been shown to have a significant therapeutic effect including in models of myocardial infarction, renal failure, transplant failure and rejection, stroke, cardiovascular disease, and autoimmune disease (e.g., Sacerdoti et al., 2005; Abraham & Kappas, 2005; Bach, 2006; Araujo et al., 2003; Liu et al., 2006; Ishikawa et al., 2001; Kruger et al., 2006; Satoh et al., 2006; Zhou et al., 2005; Morse and Choi, 2005; Morse and Choi, 2002). This enzyme breaks free heme down into iron, carbon monoxide (CO), and biliverdin (which is subsequently converted to the potent antioxidant molecule, bilirubin).

In another aspect, the synthetic triterpenoid compounds of this disclosure may be used in preventing or treating tissue damage or organ failure, acute and chronic, resulting from oxidative stress exacerbated by inflammation. Examples of diseases that fall in this category include heart failure, liver failure, transplant failure and rejection, renal failure, pancreatitis, fibrotic lung diseases (cystic fibrosis, COPD, and idiopathic pulmonary fibrosis, among others), diabetes (including complications), atherosclerosis, ischemia-reperfusion injury, glaucoma, stroke, autoimmune disease, autism, macular degeneration, and muscular dystrophy. For example, in the case of autism, studies suggest that increased oxidative stress in the central nervous system may contribute to the development of the disease (Chauhan and Chauhan, 2006).

Evidence also links oxidative stress and inflammation to the development and pathology of many other disorders of the central nervous system, including psychiatric disorders, such as psychosis, major depression, and bipolar disorder; seizure disorders, such as epilepsy; pain and sensory syndromes, such as migraine, neuropathic pain, or tinnitus; and behavioral syndromes, such as the attention deficit disorders. See, e.g., Dickerson et al., 2007; Hanson et al., 2005; Kendall-Tackett, 2007; Lencz et al., 2007; Dudhgaonkar et al., 2006; Lee et al., 2007; Morris et al., 2002; Ruster et al., 2005; McIver et al., 2005; Sarchielli et al., 2006; Kawakami et al., 2006; Ross et al., 2003, which are all incorporated by reference herein. For example, elevated levels of inflammatory cytokines, including TNF-α, interferon-γ, and IL-6, are associated with major mental illness (Dickerson et al., 2007). Microglial activation has also been linked to major mental illness. Therefore, downregulating inflammatory cytokines and inhibiting excessive activation of microglia could be beneficial in patients with schizophrenia, major depression, bipolar disorder, autism-spectrum disorders, and other neuropsychiatric disorders.

Accordingly, in pathologies involving oxidative stress alone or oxidative stress exacerbated by inflammation, treatment may comprise administering to a subject a therapeutically effective amount of a synthetic triterpenoid compound of this disclosure, such as those described above or throughout this specification. Treatment may be administered preventively, in advance of a predictable state of oxidative stress (e.g., organ transplantation or the administration of radiation therapy to a cancer patient), or it may be administered therapeutically in settings involving established oxidative stress and inflammation. In some embodiments, when a synthetic triterpenoid compound of this disclosure is used for treating a patient receiving radiation therapy and/or chemotherapy, the compound of the invention may be administered before, at the same time, and/or after the radiation or chemotherapy, or the compound may be administered in combination with the other therapies. In some embodiments, the synthetic triterpenoid compounds of this disclosure may prevent and/or reduce the severity of side effects associated with the radiation therapy or chemotherapy (using a different agent) without reducing the anticancer effects of the radiation therapy or chemotherapy. Because such side effects may be dose-limiting for the radiation therapy and/or chemotherapy, in some embodiments, the synthetic triterpenoid compounds of this disclosure may be used to allow for higher and/or more frequent dosing of the radiation therapy and/or chemotherapy, for example, resulting in greater treatment efficacy. In some embodiments, the synthetic triterpenoid compounds of this disclosure when administered in combination with the radiation therapy and/or chemotherapy may enhance the efficacy of a given dose of radiation and/or chemotherapy. In some embodiments, the synthetic triterpenoid compounds of this disclosure when administered in combination with the radiation therapy and/or chemotherapy may enhance the efficacy of a given dose of radiation and/or chemotherapy and reduce (or, at a minimum, not add to) the side effects of the radiation and/or chemotherapy. In some embodiments, and without being bound by theory, this combinatorial efficacy may result from inhibition of the activity of the pro-inflammatory transcription factor NF-κB by the compound of the invention. NF-κB is often chronically activated in cancer cells, and such activation is associated with resistance to therapy and promotion of tumor progression (e.g., Karin, 2006; Aghajan et al., 2012). Other transcription factors that promote inflammation and cancer, such as STAT3 (e.g., He and Karin 2011; Grivennikov and Karin, 2010), may also be inhibited by the synthetic triterpenoid compounds of this disclosure in some embodiments.

The synthetic triterpenoid compounds of this disclosure may be used to treat or prevent inflammatory conditions, such as sepsis, dermatitis, autoimmune disease, and osteoarthritis. The synthetic triterpenoid compounds of this disclosure may also be used to treat or prevent inflammatory pain and/or neuropathic pain, for example, by inducing Nrf2 and/or inhibiting NF-κB.

The synthetic triterpenoid compounds of this disclosure may also be used to treat or prevent diseases, such as cancer, inflammation, Alzheimer's disease, Parkinson's disease, multiple sclerosis, autism, amyotrophic lateral sclerosis, Huntington's disease, autoimmune diseases, such as rheumatoid arthritis, lupus, Crohn's disease, and psoriasis, inflammatory bowel disease, all other diseases whose pathogenesis is believed to involve excessive production of either nitric oxide or prostaglandins, and pathologies involving oxidative stress alone or oxidative stress exacerbated by inflammation. The synthetic triterpenoid compounds of this disclosure may be used in the treatment or prevention of cancers include a carcinoma, sarcoma, lymphoma, leukemia, melanoma, mesothelioma, multiple myeloma, or seminoma, or cancer of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

Another aspect of inflammation is the production of inflammatory prostaglandins, such as prostaglandin E. The synthetic triterpenoid compounds of this disclosure may be used to promote vasodilation, plasma extravasation, localized pain, elevated temperature, and other symptoms of inflammation. The inducible form of the enzyme COX-2 is associated with their production, and high levels of COX-2 are found in inflamed tissues. Consequently, inhibition of COX-2 may relieve many symptoms of inflammation and a number of important anti-inflammatory drugs (e.g., ibuprofen and celecoxib) act by inhibiting COX-2 activity. It has been demonstrated that a class of cyclopentenone prostaglandins (cyPGs) (e.g., 15-deoxy prostaglandin J2, a.k.a. PGJ2) plays a role in stimulating the orchestrated resolution of inflammation (e.g., Rajakariar et al., 2007). COX-2 is also associated with the production of cyclopentenone prostaglandins. Consequently, inhibition of COX-2 may interfere with the full resolution of inflammation, potentially promoting the persistence of activated immune cells in tissues and leading to chronic, “smoldering” inflammation. This effect may be responsible for the increased incidence of cardiovascular disease in patients using selective COX-2 inhibitors for long periods of time.

In one aspect, the synthetic triterpenoid compounds of this disclosure may be used to control the production of pro-inflammatory cytokines within the cell by selectively activating regulatory cysteine residues (RCRs) on proteins that regulate the activity of redox-sensitive transcription factors. Activation of RCRs by cyPGs has been shown to initiate a pro-resolution program in which the activity of the antioxidant and cytoprotective transcription factor Nrf2 is potently induced and the activities of the pro-oxidant and pro-inflammatory transcription factors NF-κB and the STATs are suppressed. In some embodiments, the synthetic triterpenoid compounds of this disclosure may be used to increase the production of antioxidant and reductive molecules (NQO1, HO-1, SOD1, γ-GCS) and decrease oxidative stress and the production of pro-oxidant and pro-inflammatory molecules (iNOS, COX-2, TNF-α). In some embodiments, the synthetic triterpenoid compounds of this disclosure may be used to cause the cells that host the inflammatory event to revert to a non-inflammatory state by promoting the resolution of inflammation and limiting excessive tissue damage to the host.

### A. Cancer

In some embodiments, the synthetic triterpenoid compounds of this disclosure may be used to induce apoptosis in tumor cells, to induce cell differentiation, to inhibit cancer cell proliferation, to inhibit an inflammatory response, and/or to function in a chemopreventative capacity. For example, the invention provides new polymorphic forms that have one or more of the following properties: (1) an ability to induce apoptosis and differentiate both malignant and non-malignant cells, (2) an activity at sub-micromolar or nanomolar levels as an inhibitor of proliferation of many malignant or premalignant cells, (3) an ability to suppress the de novo synthesis of the inflammatory enzyme inducible nitric oxide synthase (iNOS), (4) an ability to inhibit NF-κB activation, and (5) an ability to induce the expression of heme oxygenase-1 (HO-1).

The levels of iNOS and COX-2 are elevated in certain cancers and have been implicated in carcinogenesis and COX-2 inhibitors have been shown to reduce the incidence of primary colonic adenomas in humans (Rostom et al., 2007; Brown and DuBois, 2005; Crowel et al., 2003). iNOS is expressed in myeloid-derived suppressor cells (MDSCs) (Angulo et al., 2000) and COX-2 activity in cancer cells has been shown to result in the production of prostaglandin E₂ (PGE₂), which has been shown to induce the expression of arginase in MDSCs (Sinha et al., 2007). Arginase and iNOS are enzymes that utilize L-arginine as a substrate and produce L-ornithine and urea, and L-citrulline and NO, respectively. The depletion of arginine from the tumor microenvironment by MDSCs, combined with the production of NO and peroxynitrite has been shown to inhibit proliferation and induce apoptosis of T cells (Bronte et al., 2003). Inhibition of COX-2 and iNOS has been shown to reduce the accumulation of MDSCs, restore cytotoxic activity of tumor-associated T cells, and delay tumor growth (Sinha et al., 2007; Mazzoni et al., 2002; Zhou et al., 2007).

Inhibition of the NF-κB and JAK/STAT signaling pathways has been implicated as a strategy to inhibit proliferation of cancer epithelial cells and induce their apoptosis. Activation of STAT3 and NF-κB has been shown to result in suppression of apoptosis in cancer cells, and promotion of proliferation, invasion, and metastasis. Many of the target genes involved in these processes have been shown to be transcriptionally regulated by both NF-κB and STAT3 (Yu et al., 2007).

In addition to their direct roles in cancer epithelial cells, NF-κB and STAT3 also have important roles in other cells found within the tumor microenvironment. Experiments in animal models have demonstrated that NF-κB is required in both cancer cells and hematopoeitic cells to propagate the effects of inflammation on cancer initiation and progression (Greten et al., 2004). NF-κB inhibition in cancer and myeloid cells reduces the number and size, respectively, of the resultant tumors. Activation of STAT3 in cancer cells results in the production of several cytokines (IL-6, IL-10) which suppress the maturation of tumor-associated dendritic cells (DC). Furthermore, STAT3 is activated by these cytokines in the dendritic cells themselves. Inhibition of STAT3 in mouse models of cancer restores DC maturation, promotes antitumor immunity, and inhibits tumor growth (Kortylewski et al., 2005). In some embodiments, the synthetic triterpenoid compounds of this disclosure can be used to treat cancer, including, for example, prostate cancer. In some embodiments, the synthetic triterpenoid compounds of this disclosure can be used in a combination therapy to treat cancer including, for example, prostate cancer. See, e.g., Example H of WO 2013/163344.

### B. Multiple Sclerosis and Other Neurodegenerative Conditions

In some embodiments, the synthetic triterpenoid compounds of this disclosure may be used for treating patients for multiple sclerosis (MS) or other neurodegenerative conditions such as Alzheimer's disease, Parkinson's disease, or amyotrophic lateral sclerosis. MS is known to be an inflammatory condition of the central nervous system (Williams et al., 1994; Merrill and Benvenist, 1996; Genain and Nauser, 1997). Based on several investigations, evidence suggests that inflammatory, oxidative, and/or immune mechanisms are involved in the pathogenesis of Alzheimer's disease (AD), Parkinson's disease (PD), amyotrophic lateral sclerosis (ALS), and MS (Bagasra et al., 1995; McGeer and McGeer, 1995; Simonian and Coyle, 1996; Kaltschmidt et al., 1997). Epidemiologic data indicate that chronic use of NSAIDs which block synthesis of prostaglandins from arachidonate, markedly lowers the risk for development of AD (McGeer et al., 1996; Stewart et al., 1997). Thus, agents that block formation of NO and prostaglandins, may be used in approaches to prevent and treat neurodegenerative diseases. Successful therapeutic candidates for treating such a disease typically require an ability to penetrate the blood-brain barrier. See, for example, U.S. Patent Publication 2009/0060873, which is incorporated by reference herein.

### C. Neuroinflammation

In some embodiments, the synthetic triterpenoid compounds of this disclosure may be used for treating patients with neuroinflammation. Neuroinflammation encapsulates the idea that microglial and astrocytic responses and actions in the central nervous system have a fundamentally inflammation-like character, and that these responses are central to the pathogenesis and progression of a wide variety of neurological disorders. These ideas have been extended from Alzheimer's disease to other neurodegenerative diseases (Eikelenboom et al., 2002; Ishizawa and Dickson, 2001), to ischemic/toxic diseases (Gehrmann et al., 1995; Touzani et al., 1999), to tumor biology (Graeber et al., 2002) and even to normal brain development. Neuroinflammation incorporates a wide spectrum of complex cellular responses that include activation of microglia and astrocytes and induction of cytokines, chemokines, complement proteins, acute phase proteins, oxidative injury, and related molecular processes, and the events may have detrimental effects on neuronal function, leading to neuronal injury, further glial activation, and ultimately neurodegeneration.

### D. Renal Diseases

In some embodiments, the synthetic triterpenoid compounds of this disclosure may be used for treating patients with renal diseases and disorders, including renal failure and chronic kidney disease (CKD), based, for example, on the methods taught by U.S. Pat. No. 8,129,429, which is incorporated by reference herein. Renal failure, resulting in inadequate clearance of metabolic waste products from the blood and abnormal concentrations of electrolytes in the blood, is a significant medical problem throughout the world, especially in developed countries. Diabetes and hypertension are among the most important causes of chronic renal failure, also known as chronic kidney disease (CKD), but it is also associated with other conditions such as lupus. Acute renal failure may arise from exposure to certain drugs (e.g., acetaminophen) or toxic chemicals, or from ischemia-reperfusion injury associated with shock or surgical procedures, such as transplantation, and may result in chronic renal failure. In many patients, renal failure advances to a stage in which the patient requires regular dialysis or kidney transplantation to continue living. Both of these procedures are highly invasive and associated with significant side effects and quality of life issues. Although there are effective treatments for some complications of renal failure, such as hyperparathyroidism and hyperphosphatemia, no available treatment has been shown to halt or reverse the underlying progression of renal failure. Thus, agents that can improve compromised renal function would represent a significant advance in the treatment of renal failure.

Inflammation contributes significantly to the pathology of CKD. There is also a strong mechanistic link between oxidative stress and renal dysfunction. The NF-κB signaling pathway plays an important role in the progression of CKD as NF-κB regulates the transcription of MCP-1, a chemokine that is responsible for the recruitment of monocytes/macrophages resulting in an inflammatory response that ultimately injures the kidney (Wardle, 2001). The Keap1/Nrf2/ARE pathway controls the transcription of several genes encoding antioxidant enzymes, including heme oxygenase-1 (HO-1). Ablation of the Nrf2 gene in female mice results in the development of lupus-like glomerular nephritis (Yoh et al., 2001). Furthermore, several studies have demonstrated that HO-1 expression is induced in response to renal damage and inflammation and that this enzyme and its products—bilirubin and carbon monoxide—play a protective role in the kidney (Nath et al., 2006).

Acute kidney injury (AKI) can occur following ischemia-reperfusion, treatment with certain pharmacological agents, such as cisplatin and rapamycin, and intravenous injection of radiocontrast media used in medical imaging. As in CKD, inflammation and oxidative stress contribute to the pathology of AKI. The molecular mechanisms underlying radiocontrast-induced nephropathy (RCN) are not well understood; however, it is likely that a combination of events including prolonged vasoconstriction, impaired kidney autoregulation, and direct toxicity of the contrast media all contribute to renal failure (Tumlin et al., 2006). Vasoconstriction results in decreased renal blood flow and causes ischemia-reperfusion and the production of reactive oxygen species. HO-1 is strongly induced under these conditions and has been demonstrated to prevent ischemia-reperfusion injury in several different organs, including the kidney (Nath et al., 2006). Specifically, induction of HO-1 has been shown to be protective in a rat model of RCN (Goodman et al., 2007). Reperfusion also induces an inflammatory response, in part though activation of NF-κB signaling (Nichols, 2004). Targeting NF-κB has been proposed as a therapeutic strategy to prevent organ damage (Zingarelli et al., 2003).

### E. Cardiovascular Disease

In some embodiments, synthetic triterpenoid compounds may be used according to the methods of this invention for treating patients with cardiovascular disease. The etiology of CV disease is complex, but the majority of causes are related to inadequate or completely disrupted supply of blood to a critical organ or tissue. Frequently such a condition arises from the rupture of one or more atherosclerotic plaques, which leads to the formation of a thrombus that blocks blood flow in a critical vessel.

In some incidences, atherosclerosis may be so extensive in critical blood vessels that stenosis (narrowing of the arteries) develops and blood flow to critical organs (including the heart) is chronically insufficient. Such chronic ischemia can lead to end-organ damage of many kinds, including the cardiac hypertrophy associated with congestive heart failure.

Atherosclerosis, the underlying defect leading to many forms of cardiovascular disease, occurs when a physical defect or injury to the lining (endothelium) of an artery triggers an inflammatory response involving the proliferation of vascular smooth muscle cells and the infiltration of leukocytes into the affected area. Ultimately, a complicated lesion known as an atherosclerotic plaque may form, composed of the above-mentioned cells combined with deposits of cholesterol-bearing lipoproteins and other materials (e.g., Hansson et al., 2006). Despite the significant benefits offered by current therapeutic treatments, mortality from cardiovascular disease remains high and significant unmet needs in the treatment of cardiovascular disease remain.

Induction of HO-1 has been shown to be beneficial in a variety of models of cardiovascular disease, and low levels of HO-1 expression have been clinically correlated with elevated risk of CV disease. The synthetic triterpenoid compounds of this disclosure and methods of the invention, therefore, may be used in treating or preventing a variety of cardiovascular disorders including but not limited to atherosclerosis, hypertension, myocardial infarction, chronic heart failure, stroke, subarachnoid hemorrhage, and restenosis. In some embodiments, the synthetic triterpenoid compounds of this disclosure and methods of the invention may be used as a combination therapy with other known cardiovascular therapies such as but not limited to anticoagulants, thrombolytics, streptokinase, tissue plasminogen activators, surgery, coronary artery bypass grafting, balloon angioplasty, the use of stents, drugs which inhibit cell proliferation, or drugs which lower cholesterol levels.

### F. Diabetes

In some embodiments, the synthetic triterpenoid compounds of this disclosure may be used for treating patients with diabetes, based, for example, on the methods taught by U.S. Pat. No. 8,129,429, which is incorporated by reference herein. Diabetes is a complex disease characterized by the body's failure to regulate circulating levels of glucose. This failure may result from a lack of insulin, a peptide hormone that regulates both the production and absorption of glucose in various tissues. Deficient insulin compromises the ability of muscle, fat, and other tissues to absorb glucose properly, leading to hyperglycemia (abnormally high levels of glucose in the blood). Most commonly, such insulin deficiency results from inadequate production in the islet cells of the pancreas. In the majority of cases this arises from autoimmune destruction of these cells, a condition known as type 1 or juvenile-onset diabetes, but may also be due to physical trauma or some other cause.

Diabetes may also arise when muscle and fat cells become less responsive to insulin and do not absorb glucose properly, resulting in hyperglycemia. This phenomenon is known as insulin resistance, and the resulting condition is known as type 2 diabetes. Type 2 diabetes, the most common type, is highly associated with obesity and hypertension. Obesity is associated with an inflammatory state of adipose tissue that is thought to play a major role in the development of insulin resistance (e.g., Hotamisligil, 2006; Guilherme et al., 2008).

Diabetes is associated with damage to many tissues, largely because hyperglycemia (and hypoglycemia, which can result from excessive or poorly timed doses of insulin) is a significant source of oxidative stress. Because of their ability to protect against oxidative stress, particularly by the induction of HO-1 expression, the synthetic triterpenoid compounds of this disclosure may be used in treatments for many complications of diabetes. As noted above (Cai et al., 2005), chronic inflammation and oxidative stress in the liver are suspected to be primary contributing factors in the development of type 2 diabetes. Furthermore, PPAR_{γ} agonists such as thiazolidinediones are capable of reducing insulin resistance and are known to be effective treatments for type 2 diabetes. In some embodiments, the synthetic triterpenoid compounds of this disclosure may be used as combination therapies with PPAR_{γ} agonists such as thiazolidinediones.

### G. Arthritis

In some embodiments, the synthetic triterpenoid compounds of this disclosure and methods of this invention may be used for treating patients with a form of arthritis. In some embodiments, the forms of arthritis that could be treated with the synthetic triterpenoid compounds of this disclosure are rheumatoid arthritis (RA), psoriatic arthritis (PsA), spondyloarthropathies (SpAs) including ankylosing spondylitis (AS), reactive arthritis (ReA), and enteropathic arthritis (EA), juvenile rheumatoid arthritis (JRA), and early inflammatory arthritis.

For rheumatoid arthritis, the first signs typically appear in the synovial lining layer, with proliferation of synovial fibroblasts and their attachment to the articular surface at the joint margin (Lipsky, 1998). Subsequently, macrophages, T cells and other inflammatory cells are recruited into the joint, where they produce a number of mediators, including the cytokines interleukin-1 (IL-1), which contributes to the chronic sequelae leading to bone and cartilage destruction, and tumor necrosis factor (TNF-α), which plays a role in inflammation (Dinarello, 1998; Arend and Dayer, 1995; van den Berg, 2001). The concentration of IL-1 in plasma is significantly higher in patients with RA than in healthy individuals and, notably, plasma IL-1 levels correlate with RA disease activity (Eastgate et al., 1988). Moreover, synovial fluid levels of IL-1 are correlated with various radiographic and histologic features of RA (Kahle et al., 1992; Rooney et al., 1990).

Other forms of arthritis include psoriatic arthritis (PsA), which is a chronic inflammatory arthropathy characterized by the association of arthritis and psoriasis. Studies have revealed that PsA shares a number of genetic, pathogenic and clinical features with other spondyloarthropathies (SpAs), a group of diseases that comprise ankylosing spondylitis, reactive arthritis and enteropathic arthritis (Wright, 1979). The notion that PsA belongs to the SpA group has recently gained further support from imaging studies demonstrating widespread enthesitis in PsA but not RA (McGonagle et al., 1999; McGonagle et al., 1998). More specifically, enthesitis has been postulated to be one of the earliest events occurring in the SpAs, leading to bone remodeling and ankylosis in the spine, as well as to articular synovitis when the inflamed entheses are close to peripheral joints. Increased amounts of TNF-α have been reported in both psoriatic skin (Ettehadi et al., 1994) and synovial fluid (Partsch et al., 1997). Recent trials have shown a positive benefit of anti-TNF treatment in both PsA (Mease et al., 2000) and ankylosing spondylitis (Brandt et al., 2000).

Juvenile rheumatoid arthritis (JRA), a term for the most prevalent form of arthritis in children, is applied to a family of illnesses characterized by chronic inflammation and hypertrophy of the synovial membranes. The term overlaps, but is not completely synonymous, with the family of illnesses referred to as juvenile chronic arthritis and/or juvenile idiopathic arthritis in Europe.

Polyarticular JRA is a distinct clinical subtype characterized by inflammation and synovial proliferation in multiple joints (four or more), including the small joints of the hands (Jarvis, 2002). This subtype of JRA may be severe, because of both its multiple joint involvement and its capacity to progress rapidly over time. Although clinically distinct, polyarticular JRA is not homogeneous, and patients vary in disease manifestations, age of onset, prognosis, and therapeutic response. These differences very likely reflect a spectrum of variation in the nature of the immune and inflammatory attack that can occur in this disease (Jarvis, 1998).

Ankylosing spondylitis (AS) is a disease subset within a broader disease classification of spondyloarthropathy. Patients affected with the various subsets of spondyloarthropathy have disease etiologies that are often very different, ranging from bacterial infections to inheritance. Yet, in all subgroups, the end result of the disease process is axial arthritis.

AS is a chronic systemic inflammatory rheumatic disorder of the axial skeleton with or without extraskeletal manifestations. Sacroiliac joints and the spine are primarily affected, but hip and shoulder joints, and less commonly peripheral joints or certain extra-articular structures such as the eye, vasculature, nervous system, and gastrointestinal system may also be involved. The disease's etiology is not yet fully understood (Wordsworth, 1995; Calin and Taurog, 1998). The etiology is strongly associated with the major histocompatibility class I (MHC I) HLA-B27 allele (Calin and Taurog, 1998). AS affects individuals in the prime of their life and is feared because of its potential to cause chronic pain and irreversible damage of tendons, ligaments, joints, and bones (Brewerton et al., 1973a; Brewerton et al., 1973b; Schlosstein et al., 1973).

### H. Ulcerative Colitis

In some embodiments, the synthetic triterpenoid compounds of this disclosure and methods of this invention may be used for treating patients with ulcerative colitis. Ulcerative colitis is a disease that causes inflammation and sores, called ulcers, in the lining of the large intestine. The inflammation usually occurs in the rectum and lower part of the colon, but it may affect the entire colon. Ulcerative colitis may also be called colitis or proctitis. The inflammation makes the colon empty frequently, causing diarrhea. Ulcers form in places where the inflammation has killed the cells lining the colon and the ulcers bleed and produce pus.

Ulcerative colitis is an inflammatory bowel disease (IBD), the general name for diseases that cause inflammation in the small intestine and colon. Ulcerative colitis can be difficult to diagnose because its symptoms are similar to other intestinal disorders and to another type of IBD, Crohn's disease. Crohn's disease differs from ulcerative colitis because it causes inflammation deeper within the intestinal wall. Also, Crohn's disease usually occurs in the small intestine, although the disease can also occur in the mouth, esophagus, stomach, duodenum, large intestine, appendix, and anus.

### I. Crohn's Disease

In some embodiments, the synthetic triterpenoid compounds of this disclosure and methods of this invention may be used for treating patients with Crohn's disease. Crohn's disease symptoms include intestinal inflammation and the development of intestinal stenosis and fistulas; neuropathy often accompanies these symptoms. Anti-inflammatory drugs, such as 5-aminosalicylates (e.g., mesalamine) or corticosteroids, are typically prescribed, but are not always effective (reviewed in Botoman et al., 1998). Immunosuppression with cyclosporine is sometimes beneficial for patients resistant to or intolerant of corticosteroids (Brynskov et al., 1989).

In active cases of Crohn's disease, elevated concentrations of TNF-α and IL-6 are secreted into the blood circulation, and TNF-α, IL-1, IL-6, and IL-8 are produced in excess locally by mucosal cells (id.; Funakoshi et al., 1998). These cytokines can have far-ranging effects on physiological systems including bone development, hematopoiesis, and liver, thyroid, and neuropsychiatric function. Also, an imbalance of the IL-1β/IL-1ra ratio, in favor of pro-inflammatory IL-10, has been observed in patients with Crohn's disease (Rogler and Andus, 1998; Saiki et al., 1998; Dionne et al., 1998; but see Kuboyama, 1998).

Treatments that have been proposed for Crohn's disease include the use of various cytokine antagonists (e.g., IL-1ra), inhibitors (e.g., of IL-10 converting enzyme and antioxidants) and anti-cytokine antibodies (Rogler and Andus, 1998; van Hogezand and Verspaget, 1998; Reimund et al., 1998; Lugering et al., 1998; McAlindon et al., 1998). In particular, monoclonal antibodies against TNF-α have been tried with some success in the treatment of Crohn's disease (Targan et al., 1997; Stack et al., 1997; van Dullemen et al., 1995). These compounds may be used in combination therapy with RTA 408, the polymorphic forms, and methods of the present disclosure.

### J. Systemic Lupus Erythematosus

In some embodiments, the synthetic triterpenoid compounds of this disclosure and methods of this invention may be used for treating patients with SLE. Systemic lupus erythematosus (SLE) is an autoimmune rheumatic disease characterized by deposition in tissues of autoantibodies and immune complexes leading to tissue injury (Kotzin, 1996). In contrast to autoimmune diseases, such as MS and type 1 diabetes mellitus, SLE potentially involves multiple organ systems directly, and its clinical manifestations are diverse and variable (reviewed by Kotzin and O'Dell, 1995). For example, some patients may demonstrate primarily skin rash and joint pain, show spontaneous remissions, and require little medication. At the other end of the spectrum are patients who demonstrate severe and progressive kidney involvement that requires therapy with high doses of steroids and cytotoxic drugs such as cyclophosphamide (Kotzin, 1996).

One of the antibodies produced by SLE, IgG anti-dsDNA, plays a major role in the development of lupus glomerulonephritis (GN) (Hahn and Tsao, 1993; Ohnishi et al., 1994). Glomerulonephritis is a serious condition in which the capillary walls of the kidney's blood purifying glomeruli become thickened by accretions on the epithelial side of glomerular basement membranes. The disease is often chronic and progressive and may lead to eventual renal failure.

### K. Irritable Bowel Syndrome

In some embodiments, the synthetic triterpenoid compounds of this disclosure and methods of this invention may be used for treating patients with irritable bowel syndrome (IBS). IBS is a functional disorder characterized by abdominal pain and altered bowel habits. This syndrome may begin in young adulthood and can be associated with significant disability. This syndrome is not a homogeneous disorder. Rather, subtypes of IBS have been described on the basis of the predominant symptom—diarrhea, constipation, or pain. In the absence of “alarm” symptoms, such as fever, weight loss, and gastrointestinal bleeding, a limited workup is needed.

Increasingly, evidence for the origins of IBS suggests a relationship between infectious enteritis and subsequent development of IBS. Inflammatory cytokines may play a role. In a survey of patients with a history of confirmed bacterial gastroenteritis (Neal et al., 1997), 25% reported persistent alteration of bowel habits. Persistence of symptoms may be due to psychological stress at the time of acute infection (Gwee et al., 1999).

Recent data suggest that bacterial overgrowth in the small intestine may also have a role in IBS symptoms. In one study (Pimentel et al., 2000), 157 (78%) of 202 IBS patients referred for hydrogen breath testing had test findings that were positive for bacterial overgrowth. Of the 47 subjects who had follow-up testing, 25 (53%) reported improvement in symptoms (i.e., abdominal pain and diarrhea) with antibiotic treatment.

### L. Sjögren's Syndrome

In some embodiments, the synthetic triterpenoid compounds of this disclosure and methods of this invention may be used for treating patients with Sjögren's syndrome. Primary Sjögren's syndrome (SS) is a chronic, slowly progressive, systemic autoimmune disease, which affects predominantly middle-aged women (female-to-male ratio 9:1), although it can be seen in all ages including childhood (Jonsson et al., 2002). The disease is characterized by lymphocytic infiltration and destruction of the exocrine glands, which are infiltrated by mononuclear cells including CD4+, CD8+ lymphocytes, and B-cells (Jonsson et al., 2002). In addition, extraglandular (systemic) manifestations are seen in one-third of patients (Jonsson et al., 2001).

In other systemic autoimmune diseases, such as RA, factors critical for ectopic germinal centers (GCs) have been identified. Rheumatoid synovial tissues with GCs were shown to produce chemokines CXCL13, CCL21, and lymphotoxin (LT)-β (detected on follicular center and mantle zone B cells). Multivariate regression analysis of these analytes identified CXCL13 and LT-β as the solitary cytokines predicting GCs in rheumatoid synovitis (Weyand and Goronzy, 2003). Recently CXCL13 and CXCR5 in salivary glands has been shown to play an essential role in the inflammatory process by recruiting B and T cells, therefore contributing to lymphoid neogenesis and ectopic GC formation in SS (Salomonsson et al., 2002).

### M. Psoriasis

In some embodiments, the synthetic triterpenoid compounds of this disclosure and methods of this invention may be used for treating patients with psoriasis. Psoriasis is a chronic skin disease of scaling and inflammation that affects 2 to 2.6 percent of the United States population, or between 5.8 and 7.5 million people. Psoriasis occurs when skin cells quickly rise from their origin below the surface of the skin and pile up on the surface before they have a chance to mature. Usually this movement (also called turnover) takes about a month, but in psoriasis turnover may occur in only a few days. In its typical form, psoriasis results in patches of thick, red (inflamed) skin covered with silvery scales. These patches, which are sometimes referred to as plaques, usually itch or feel sore. The plaques most often occur on the elbows, knees, other parts of the legs, scalp, lower back, face, palms, and soles of the feet, but they can occur on skin anywhere on the body. The disease may also affect the fingernails, the toenails, and the soft tissues of the genitals and inside the mouth.

Psoriasis is a skin disorder driven by the immune system, especially involving a type of white blood cell called a T cell. Normally, T cells help protect the body against infection and disease. In the case of psoriasis, T cells are put into action by mistake and become so active that they trigger other immune responses, which lead to inflammation and to rapid turnover of skin cells.

N. Infectious diseases

In some embodiments, the synthetic triterpenoid compounds of this disclosure and methods of the present disclosure may be useful in the treatment of infectious diseases, including viral and bacterial infections. As noted above, such infections may be associated with severe localized or systemic inflammatory responses. For example, influenza and SARS-CoV-2 may cause severe inflammation of the lung and bacterial infection can cause the systemic hyperinflammatory response, including the excessive production of multiple inflammatory cytokines, which is the hallmark of sepsis. In addition, compounds of the invention may be useful in directly inhibiting the replication of viral pathogens. Previous studies have demonstrated that related compounds such as CDDO can inhibit the replication of HIV in macrophages (Vazquez et al., 2005). Other studies have indicated that inhibition of NF-κB signaling may inhibit influenza virus replication, and that cyclopentenone prostaglandins may inhibit viral replication (e.g., Mazur et al., 2007; Pica et al., 2000).

The present invention relates to the treatment or prevention of each of the diseases/disorders/conditions referred to above using the synthetic triterpenoid compounds of this disclosure or pharmaceutically acceptable salts thereof, or a polymorphic form of such compounds, or a pharmaceutical composition comprising any of the aforementioned entities and a pharmaceutically acceptable carrier (including, e.g., the pharmaceutical compositions described above).

### V. Pharmaceutical Formulations and Routes of Administration

Administration of the compounds of the present invention to a patient will follow general protocols for the administration of pharmaceuticals, taking into account the toxicity, if any, of the drug. It is expected that the treatment cycles would be repeated as necessary.

The compounds of the present invention may be administered by a variety of methods, e.g., orally or by injection (e.g., subcutaneous, intravenous, intraperitoneal, etc.). Depending on the route of administration, the active compounds may be coated by a material to protect the compound from the action of acids and other natural conditions which may inactivate the compound. They may also be administered by continuous perfusion/infusion of a disease or wound site.

Specific examples of formulations, including a polymer-based dispersion of CDDO-Me that showed improved oral bioavailability, are provided in U.S. Patent Application Publication No. 2009/0048204, which is incorporated herein by reference in its entirety. It will be recognized by those skilled in the art that other methods of manufacture may be used to produce dispersions of the present invention with equivalent properties and utility (see, Repka et al., 2002 and references cited therein). Such alternative methods include but are not limited to solvent evaporation, extrusion, such as hot melt extrusion, and other techniques.

To administer the active compound by other than parenteral administration, it may be necessary to coat the compound with, or co-administer the compound with, a material to prevent its inactivation. For example, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The therapeutic compound may also be administered parenterally, intraperitoneally, intraocularly, intraspinally, or intracerebrally. Dispersions may be prepared in, e.g., glycerol, liquid polyethylene glycols, mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's or patient's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects or patients to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. In some embodiments, the compound may be prepared in a lotion, cream, gel, oil, ointment, salve, solution, suspension, or emulsion. Alternatively, if local delivery to the lungs is desired the therapeutic compound may be administered by inhalation in a dry-powder or aerosol formulation.

The therapeutic compound may be formulated in a biocompatible matrix for use in a drug-eluting stent.

The therapeutic compound will typically be administered at a therapeutically effective dosage sufficient to treat a condition associated with a given patient. For example, the efficacy of a compound can be evaluated in an animal model system that may be predictive of efficacy in treating the disease in humans, such as the model systems shown in the examples and drawings.

In some embodiments, the effective dose range for the therapeutic compound can be extrapolated from effective doses determined in animal studies for a variety of different animals. In general a human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present invention or composition comprising a compound of the present invention administered to a subject or a patient may be determined by physical and physiological factors such as age, sex, body weight, severity of condition, the type of disease being treated, previous or concurrent therapeutic interventions, idiopathy of the subject or the patient and on the route of administration. These factors may be determined by a skilled artisan. The practitioner responsible for administration will typically determine the concentration of active ingredient(s) in a composition and appropriate dose(s) for the individual subject or patient. The dosage may be adjusted by the individual physician in the event of any complication.

In some embodiments, the pharmaceutically effective amount is a daily dose from about 0.1 mg to about 500 mg of the compound. In some variations, the daily dose is from about 1 mg to about 300 mg of the compound. In some variations, the daily dose is from about 10 mg to about 200 mg of the compound. In some variations, the daily dose is about 25 mg of the compound. In other variations, the daily dose is about 75 mg of the compound. In still other variations, the daily dose is about 150 mg of the compound. In further variations, the daily dose is from about 0.1 mg to about 30 mg of the compound. In some variations, the daily dose is from about 0.5 mg to about 20 mg of the compound. In some variations, the daily dose is from about 1 mg to about 15 mg of the compound. In some variations, the daily dose is from about 1 mg to about 10 mg of the compound. In some variations, the daily dose is from about 1 mg to about 5 mg of the compound.

In some embodiments, the pharmaceutically effective amount is a daily dose of 0.01-25 mg of compound per kg of body weight. In some variations, the daily dose is 0.05-20 mg of compound per kg of body weight. In some variations, the daily dose is 0.1-10 mg of compound per kg of body weight. In some variations, the daily dose is 0.1-5 mg of compound per kg of body weight. In some variations, the daily dose is 0.1-2.5 mg of compound per kg of body weight.

In some embodiments, the pharmaceutically effective amount is a daily dose of 0.1-1000 mg of compound per kg of body weight. In some variations, the daily dose is 0.15-20 mg of compound per kg of body weight. In some variations, the daily dose is 0.20-10 mg of compound per kg of body weight. In some variations, the daily dose is 0.40-3 mg of compound per kg of body weight. In some variations, the daily dose is 0.50-9 mg of compound per kg of body weight. In some variations, the daily dose is 0.60-8 mg of compound per kg of body weight. In some variations, the daily dose is 0.70-7 mg of compound per kg of body weight. In some variations, the daily dose is 0.80-6 mg of compound per kg of body weight. In some variations, the daily dose is 0.90-5 mg of compound per kg of body weight. In some variations, the daily dose is from about 1 mg to about 5 mg of compound per kg of body weight.

The effective amount may be less than 1 mg/kg/day, less than 500 mg/kg/day, less than 250 mg/kg/day, less than 100 mg/kg/day, less than 50 mg/kg/day, less than 25 mg/kg/day, or less than 10 mg/kg/day. It may alternatively be in the range of 1 mg/kg/day to 200 mg/kg/day. In some embodiments, the amount could be 10, 30, 100, or 150 mg/kg formulated as a suspension in sesame oil. In some embodiments, the amount could be 3, 10, 30 or 100 mg/kg administered daily via oral gavage. In some embodiments, the amount could be 10, 30, or 100 mg/kg administered orally. For example, regarding treatment of diabetic patients, the unit dosage may be an amount that reduces blood glucose by at least 40% as compared to an untreated patient. In another embodiment, the unit dosage is an amount that reduces blood glucose to a level that is ±10% of the blood glucose level of a non-diabetic patient.

An effective amount typically will vary from about 0.001 mg/kg to about 1,000 mg/kg, from about 0.01 mg/kg to about 750 mg/kg, from about 0.1 mg/kg to about 500 mg/kg, from about 0.2 mg/kg to about 250 mg/kg, from about 0.3 mg/kg to about 150 mg/kg, from about 0.3 mg/kg to about 100 mg/kg, from about 0.4 mg/kg to about 75 mg/kg, from about 0.5 mg/kg to about 50 mg/kg, from about 0.6 mg/kg to about 30 mg/kg, from about 0.7 mg/kg to about 25 mg/kg, from about 0.8 mg/kg to about 15 mg/kg, from about 0.9 mg/kg to about 10 mg/kg, from about 1 mg/kg to about 5 mg/kg, from about 100 mg/kg to about 500 mg/kg, from about 1.0 mg/kg to about 250 mg/kg, or from about 10.0 mg/kg to about 150 mg/kg, in one or more dose administrations daily, for one or several days (depending, of course, of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some particular embodiments, the amount is less than 10,000 mg per day with a range, for example, of 750 mg to 9,000 mg per day.

The effective amount may be less than 1 mg/kg/day, less than 500 mg/kg/day, less than 250 mg/kg/day, less than 100 mg/kg/day, less than 50 mg/kg/day, less than 25 mg/kg/day, less than 10 mg/kg/day, or less than 5 mg/kg/day. It may alternatively be in the range of 1 mg/kg/day to 200 mg/kg/day. For example, regarding treatment of patients with Alport syndrome, the unit dosage may be an amount that reduces urine protein concentration by at least 40% as compared to an untreated subject or patient. In another embodiment, the unit dosage is an amount that reduces urine protein concentration to a level that is within ±10% of the urine protein level of a healthy subject or patient.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 1 mg/kg/body weight to about 5 mg/kg/body weight, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present invention may comprise, for example, at least about 0.1% of a compound of the present invention. In other embodiments, the compound of the present invention may comprise between about 2% to about 75% of the weight of the unit, or between about 25% to about 60%, for example, and any range derivable therein.

In certain embodiments, a pharmaceutical composition of the present disclosure may comprise, for example, at least about 0.01% of RTA 408. In other embodiments, RTA 408 may comprise between about 0.01% to about 75% of the weight of the unit, or between about 0.01% to about 5%, for example, and any range derivable therein. In some embodiments, RTA 408 may be used in a formulation such as a suspension in sesame oil of 0.01%, 0.1%, or 1%. In some embodiments, RTA 408 may be formulated for topical administration to the skin or eye, using a pharmaceutically suitable carrier or as a suspension, emulsion, or solution in concentrations ranging from about 0.01% to 10%. In some embodiments the concentration ranges from about 0.1% to about 5%. The optimal concentration may vary depending upon the target organ, the specific preparation, and the condition to be treated.

Single or multiple doses of the agents are contemplated. Desired time intervals for delivery of multiple doses can be determined by one of ordinary skill in the art employing no more than routine experimentation. As an example, subjects or patients may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the subject or patient has eaten or will eat.

Non-limiting specific formulations include CDDO-Me polymer dispersions (see U.S. Patent Application Publication No. 2009/0048204, filed Aug. 13, 2008, which is incorporated herein by reference). Some of the formulations reported therein exhibited higher bioavailability than either the micronized Form A or nanocrystalline Form A formulations. Additionally, the polymer dispersion based formulations demonstrated further surprising improvements in oral bioavailability relative to the micronized Form B formulations. For example, the methacrylic acid copolymer, Type C and HPMC-P formulations showed the greatest bioavailability in the subject monkeys.

RTA 408 may be rendered fully amorphous using a direct spray drying procedure. RTA 408 can be orally administered, for example, with an inert diluent or an assimilable edible carrier. The therapeutic compound and other ingredients may also be enclosed in a hard or soft shell gelatin capsule, compressed into tablets, or incorporated directly into the patient's diet. For oral therapeutic administration, the therapeutic compound may be incorporated, for example, with excipients and used in the form of ingestible tablets, buccal tablets, troches, capsules including hard or soft capsules, elixirs, emulsions, solid dispersions, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

### VI. Combination Therapy

In addition to being used as monotherapy, the synthetic triterpenoids of this disclosure may also find use in combination therapies. Effective combination therapy may be achieved with a single composition or pharmacological formulation that includes both agents, or with two distinct compositions or formulations, administered at the same time, wherein one composition includes a synthetic triterpenoid of this disclosure, and the other includes the second agent(s). Alternatively, administration of the synthetic triterpenoid of this disclosure may precede or follow the other agent treatment by intervals ranging from minutes to months. In embodiments where the other agent and the synthetic triterpenoid of this disclosure are administered separately, one would generally ensure that a significant period of time did not expire between the time of each delivery, such that each agent would still be able to exert an advantageously combined effect. In such instances, it is contemplated that one would typically administer the synthetic triterpenoid of this disclosure and the other therapeutic agent within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of the synthetic triterpenoid of this disclosure, or the other agent will be desired. In this regard, various combinations may be employed. By way of illustration, where the synthetic triterpenoid of this disclosure is “A” and the other agent is “B”, the following permutations are exemplary:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is contemplated that other therapeutic agents may be used in conjunction with the treatments of the current invention. In some embodiments, the present invention contemplates the use of one or more other therapies for the treatment of Alport syndrome in conjunction with the compounds described in the methods herein. These therapies include the use of an angiotensin-converting enzyme (ACE) inhibitor, angiotensin receptor blockade (ARB), or an aldosterone antagonist. Some non-limiting examples of ACE inhibitors include Ramipril, enalapril, Lisinopril, benazepril, fosinopril, quinapril, cilazapril, perinopril, or trandolapril. Similarly, some non-limiting examples of angiotensin receptor blockade agents include losartan, candesartan, irbesartan, telmisartan, valsartan, or epresartan. A non-limiting example of an aldosterone antagonist is spirolactone.

Additionally, combination therapies for the treatment of cardiovascular disease using the synthetic triterpenoids of this disclosure are contemplated. For example, such methods may further comprise administering a pharmaceutically effective amount of one or more cardiovascular drugs in addition to a synthetic triterpenoid of this disclosure. The cardiovascular drug may be but not limited to, for example, a cholesterol lowering drug, an anti-hyperlipidemic, a calcium channel blocker, an anti-hypertensive, or an HMG-CoA reductase inhibitor. In some embodiments, non-limiting examples of cardiovascular drugs include amlodipine, aspirin, ezetimibe, felodipine, lacidipine, lercanidipine, nicardipine, nifedipine, nimodipine, nisoldipine or nitrendipine. In other embodiments, other non-limiting examples of cardiovascular drugs include atenolol, bucindolol, carvedilol, clonidine, doxazosin, indoramin, labetalol, methyldopa, metoprolol, nadolol, oxprenolol, phenoxybenzamine, phentolamine, pindolol, prazosin, propranolol, terazosin, timolol or tolazoline. In other embodiments, the cardiovascular drug may be, for example, a statin, such as atorvastatin, cerivastatin, fluvastatin, lovastatin, mevastatin, pitavastatin, pravastatin, rosuvastatin or simvastatin.

### VII. Characteristics of Patients Who should be Excluded from Treatment with Bardoxolone Methyl

Several clinical studies have shown that treatment with bardoxolone methyl improved markers of renal function (including estimated glomerular filtration rate, or eGFR), insulin resistance, and endothelial dysfunction (Pergola et al., 2011). These observations led to the initiation of a large Phase 3 trial (BEACON) of bardoxolone methyl in patients with stage 4 CKD and type 2 diabetes. The primary endpoint in the BEACON trial was a composite of progression to end-stage renal disease (ESRD) and all-cause mortality. This trial was terminated due to excess severe adverse events and mortality in the group of patients treated with bardoxolone methyl.

As discussed below, subsequent analysis of the data from the BEACON trial showed that most of the severe adverse events and mortality involved heart failure and were highly correlated with the presence of one or more risk factors including: (a) elevated baseline levels of B-type natriuretic peptide (BNP; e.g., >200 pg/mL); (b) baseline eGFR<20; (c) history of left-sided heart disease; (d) high baseline albumin-to-creatinine ratio (ACR; e.g., >300 mg/g as defined by dipstick proteinuria of 3+); and (e) advanced age (e.g., >75 years). The analysis indicated that heart failure events were likely related to the development of acute fluid overload in the first three to four weeks of bardoxolone methyl treatment and that this was potentially due to inhibition of endothelin-1 signaling in the kidney. A previous trial of an endothelin receptor antagonist in stage 4 CKD patients was terminated due to a pattern of adverse events and mortality very similar to that found in the BEACON trial. Subsequent non-clinical studies confirmed that bardoxolone methyl, at physiologically relevant concentrations, inhibits endothelin-1 expression in renal proximal tubule epithelial cells and inhibits endothelin receptor expression in human mesangial and endothelial cells. Accordingly, patients at risk of adverse events from inhibition of endothelin signaling should be excluded from future clinical use of bardoxolone methyl.

The present invention concerns new methods of treating Alport syndrome that include modification of the glomerular basement membrane as a significant contributing factor. It also concerns the preparation of pharmaceutical compositions for the treatment of such disorders. In some embodiments of the present invention, patients for treatment are selected on the basis of several criteria: (1) diagnosis of a disorder that involves endothelial dysfunction as a significant contributing factor; (2) lack of elevated levels of B-type natriuretic peptide (BNP; e.g., BNP titers must be <200 pg/mL); (3) lack of chronic kidney disease (e.g., eGFR>60) or lack of advanced chronic kidney disease (e.g., eGFR>45); (4) lack of a history of left-sided myocardial disease; and (5) lack of a high ACR (e.g., ACR below 300 mg/g). In some embodiments of the invention, patients with a diagnosis of type 2 diabetes are excluded. In some embodiments of the invention, patients with a diagnosis of cancer are excluded. In some embodiments, patients of advanced age (e.g., >75 years) are excluded. In some embodiments, patients are closely monitored for rapid weight gain suggestive of fluid overload. For example, patients may be instructed to weigh themselves daily for the first four weeks of treatment and contact the prescribing physician if increases of greater than five pounds are observed.

### A. BEACON Study

### 1. Design of Study

Study 402-C-0903, titled “Bardoxolone Methyl Evaluation in Patients with Chronic Kidney Disease and Type 2 Diabetes: The Occurrence of Renal Events” (BEACON) was a phase 3, randomized, double-blind, placebo-controlled, parallel-group, multinational, multicenter study designed to compare the efficacy and safety of bardoxolone methyl (BARD) to placebo (PBO) in patients with stage 4 chronic kidney disease and type 2 diabetes. A total of 2,185 patients were randomized 1:1 to once-daily administration of bardoxolone methyl (20 mg) or placebo. The primary efficacy endpoint of the study was the time-to-first event in the composite endpoint defined as end-stage renal disease (ESRD; need for chronic dialysis, renal transplantation, or renal death) or cardiovascular (CV) death. The study had three secondary efficacy endpoints: (1) change in estimated glomerular filtration rate (eGFR); (2) time-to-first hospitalization for heart failure or death due to heart failure; and (3) time-to-first event of the composite endpoint consisting of non-fatal myocardial infarction, non-fatal stroke, hospitalization for heart failure, or cardiovascular death.

A subset of the BEACON patients consented to additional 24-hour assessments including ambulatory blood pressure monitoring (ABPM) and 24-hour urine collections. An independent Events Adjudication Committee (EAC), blinded to study treatment assignment, evaluated whether renal events, cardiovascular events, and neurological events met the pre-specified definitions of the primary and secondary endpoints. An IDMC, consisting of external clinical experts supported by an independent statistical group, reviewed unblinded safety data throughout the study and made recommendations as appropriate.

### 2. Demographics and Baseline Characteristics of the Population

Table 4 presents summary statistics on select demographic and baseline characteristics of patients enrolled in BEACON. Demographic characteristics were comparable across the two treatment groups. In all treatment groups combined, the average age was 68.5 years and 57% of the patients were male. The bardoxolone methyl arm had slightly more patients in the age subgroup ≥75 years than the placebo arm (27% in bardoxolone methyl arm versus 24% in the placebo arm). Mean weight and BMI across both treatment groups was 95.2 kg and 33.8 kg/m², respectively. Baseline kidney function was generally similar in the two treatment groups; mean baseline eGFR, as measured by the 4-variable Modified Diet in Renal Disease (MDRD) equation, was 22.5 mL/min/1.73 m² and the geometric mean albumin/creatinine ratio (ACR) was 215.5 mg/g for the combined treatment groups.

TABLE 4
Select Demographics and Baseline Characteristics
of Bardoxolone Methyl (BARD) versus Placebo
(PBO) Patients in BEACON (ITT Population)

| | BARD | | PBO | | Total | |
|---|---|---|---|---|---|---|
| | N = 1088 | | N = 1097 | | N = 2185 | |
| Sex, n (%) | | | | | | |
| Male | 626 | (58) | 625 | (57) | 1251 | (57) |
| Female | 462 | (42) | 472 | (43) | 934 | (43) |
| Age at informed | | | | | | |
| consent (years) | | | | | | |
| n | 1088 | | 1097 | | 2185 | |
| Mean (SD) | 68.9 | (9.7) | 68.2 | (9.4) | 68.5 | (9.6) |
| Range (min, max) | 32, 92 | | 29, 93 | | 29, 93 | |
| Age subgroup, n (%) | | | | | | |
| <75 | 786 | (72) | 829 | (76) | 1615 | (74) |
| ≥75 | 302 | (27) | 268 | (24) | 570 | (26) |
| Weight (kg) | | | | | | |
| n | 1087 | | 1097 | | 2184 | |
| Mean (SD) | 95.1 | (22.0) | 95.3 | (21.1) | 95.2 | (21.5) |
| Range (min, max) | 46, 194 | | 45, 186 | | 45, 194 | |
| BMI (kg/m²) | | | | | | |
| n | 1087 | | 1097 | | 2184 | |
| Mean (SD) | 33.7 | (7.1) | 33.9 | (7.2) | 33.8 | (7.1) |
| Range (min, max) | 19, 93 | | 19, 64 | | 19, 93 | |
| eGFR (mL/min/1.73 | | | | | | |
| m²) mean (SD) | | | | | | |
| n | 1088 | | 1097 | | 2185 | |
| Mean (SD) | 22.4 | (4.3) | 22.5 | (4.6) | 22.5 | (4.5) |
| Range (min, max) | 13, 34 | | 13, 58 | | 13, 58 | |
| eGFR MDRD | | | | | | |
| subgroup, n (%) | | | | | | |
| 15-<20 | 325 | (30) | 347 | (32) | 672 | (31) |
| 20-<25 | 399 | (37) | 366 | (33) | 765 | (35) |
| 25-<30 | 311 | (29) | 318 | (29) | 629 | (29) |
| ACR (mg/g) | | | | | | |
| geometric mean | | | | | | |
| n | 1088 | | 1097 | | 2185 | |
| Geometric mean | 210.4 | | 220.7 | | 215.5 | |
| (95% CI) | (188, 236) | | (196, 249) | | (198, 234) | |
| Range (min, max) | <1, 4581 | | <1, 79466 | | <1, 79466 | |
| ACR subgroup, n (%) | | | | | | |
| <30 | 200 | (18) | 211 | (19) | 411 | (19) |
| 30-300 | 348 | (32) | 308 | (28) | 656 | (30) |
| >300 | 540 | (50) | 578 | (53) | 1118 | (51) |

Patients were administered placebo or 20 mg of bardoxolone methyl once daily.

### B. BEACON Results

1. Effect of Bardoxolone Methyl on eGFR

On average, bardoxolone methyl patients had expected increases in eGFR that occurred by Week 4 of treatment and remained above baseline through Week 48. In contrast, placebo-treated patients on average had unchanged or slight decreases from baseline. The proportion of patients with eGFR declines was markedly reduced in bardoxolone methyl versus placebo-treated patients. The eGFR trajectories and the proportions of decliners observed in BEACON after one year of treatment were consistent with modeled expectations and results from the BEAM study (RTA402-C-0804). As shown in Table 5, the number of patients who experienced a renal and urinary disorder serious adverse event (SAE) was lower in the bardoxolone methyl group than in the placebo group (52 vs. 71, respectively). Additionally, and as discussed in the following section, slightly fewer ESRD events were observed in the bardoxolone methyl group than in the placebo group. Collectively, these data suggest that bardoxolone methyl treatment did not worsen renal status acutely or over time.

TABLE 5
Incidence of Treatment-Emergent Serious Adverse Events in BEACON
within Each Primary System Organ Class (Safety Population)

| | | | Bardoxolone | |
|---|---|---|---|---|
| | Placebo | | methyl | |
| | N = 1093 | | N = 1092 | |
| MedDRA System Organ Class | n (%) | | n (%) | |
| Patients with any serious adverse event | 295 | (27) | 363 | (33) |
| Number of serious adverse events | 557 | | 717 | |
| Cardiac disorders | 84 | (8) | 124 | (11) |
| Infections and infestations | 63 | (6) | 79 | (7) |
| Renal and urinary disorders | 71 | (6) | 52 | (5) |
| Metabolism and nutrition disorders | 42 | (4) | 51 | (5) |
| Gastrointestinal disorders | 39 | (4) | 46 | (4) |
| Respiratory, thoracic and mediastinal | 32 | (3) | 43 | (4) |
| disorders | | | | |
| Nervous system disorders | 35 | (3) | 37 | (3) |
| General disorders and administration site | 20 | (2) | 29 | (3) |
| conditions | | | | |
| Vascular disorders | 18 | (2) | 20 | (2) |
| Injury, poisoning and procedural | 17 | (2) | 19 | (2) |
| complications | | | | |
| Musculoskeletal and connective tissue | 13 | (1) | 21 | (2) |
| disorders | | | | |
| Blood and lymphatic system disorders | 11 | (1) | 20 | (2) |
| Neoplasms benign, malignant and | 10 | (1) | 11 | (1) |
| unspecified (incl. cysts and polyps) | | | | |
| Hepatobiliary disorders | 8 | (1) | 4 | (<1) |
| Psychiatric disorders | 3 | (<1) | 3 | (<1) |
| Eye disorders | 2 | (<1) | 3 | (<1) |
| Investigations | 2 | (<1) | 3 | (<1) |
| Reproductive system and breast disorders | 3 | (<1) | 2 | (<1) |
| Skin and subcutaneous tissue disorders | 1 | (<1) | 4 | (<1) |
| Ear and labyrinth disorders | 1 | (<1) | 3 | (<1) |
| Endocrine disorders | 1 | (<1) | 1 | (<1) |

| | | | |
|---|---|---|---|
| Immune system disorders | 0 | 2 | (<1) |
| Surgical and medical procedures | 0 | 2 | (<1) |

Table includes only serious adverse events with onset more than 30 days after a patient's last dose of study drug. Column header counts and denominators are the number of patients in the safety population. Each patient is counted at most once in each System Organ Class and Preferred Term.

### 2. Primary Composite Outcome in BEACON

Table 6 provides a summary of adjudicated primary endpoints that occurred on or before the date of study termination (Oct. 18, 2012). Despite the slight reduction in the number of ESRD events in the bardoxolone methyl vs. placebo treatment groups, the number of composite primary endpoints was equal in the two treatment groups (HR=0.98) due to a slight increase in cardiovascular death events, as depicted in plots of time-to-first composite primary event analysis.

TABLE 6
Adjudicated Primary Endpoints in Bardoxolone Methyl (BARD)
vs. Placebo (PBO) Patients in BEACON (ITT Population)

| | PBO | | BARD | | Hazard | |
|---|---|---|---|---|---|---|
| | N = 1097 | | N = 1088 | | ratio | p- |
| | n (%) | | n (%) | | (95% CI)^{a} | value^{b} |
| Composite primary | 69 | (6) | 69 | (6) | 0.98 (0.70, | 0.92 |
| efficacy outcome | | | | | 1.37) | |
| Patient's first event | | | | | | |
| End stage renal | 51 | (5) | 43 | (4) | | |
| disease (ESRD) | | | | | | |
| Chronic dialysis | 47 | (4) | 40 | (4) | | |
| Renal transplant | 3 | (<1) | 1 | (<1) | | |
| Renal death | 1 | (<1) | 2 | (<1) | | |
| CV death | 18 | (2) | 26 | (2) | | |

^{a}Hazard ratio (bardoxolone methyl/placebo) and 95% confidence interval (CI) were estimated using a Cox proportional hazards model with treatment group, continuous baseline eGFR, and continuous baseline log ACR as covariates. Breslow's method of handling ties in event time was used.
^{b}Treatment group comparisons used SAS's Type 3 chi-square test and two-sided p-value associated with the treatment group variable in the Cox proportional hazards model.

### C. Effects of Bardoxolone Methyl on Heart Failure and Blood Pressure

1. Adjudicated Heart Failure in BEACON The data in Table 7 present a post-hoc analysis of demographic and select laboratory parameters of BEACON patients stratified by treatment group and occurrence of an adjudicated heart failure event. The number of patients with heart failure includes all events through last date of contact (ITT Population).

Comparison of baseline characteristics of patients with adjudicated heart failure events revealed that both bardoxolone methyl-treated and placebo-treated patients with heart failure were more likely to have had a prior history of cardiovascular disease and heart failure and had higher baseline values for B-type natriuretic peptide (BNP) and QTc interval with Fredericia correction (QTcF). Even though the risk for heart failure was higher in the bardoxolone methyl-treated patients, these data suggest that development of heart failure in both groups appeared to be associated with traditional risk factors for heart failure. Baseline ACR was significantly higher in bardoxolone methyl-treated patients with heart failure events than those without. Also of note, the mean baseline level of BNP in patients who experienced heart failure in both treatment groups was meaningfully elevated and suggested that these patients were likely retaining fluid and in sub-clinical heart failure prior to randomization.

TABLE 7
Select Demographic and Baseline Characteristics for Bardoxolone
Methyl vs. Placebo Patients Stratified by Heart Failure Status

| | With Heart | | Without Heart | | | |
|---|---|---|---|---|---|---|
| | Failure | | Failure | | Total | |
| | BARD | PBO | BARD | PBO | BARD | PBO |
| Patients | (N = 103) | (N = 57) | (N = 985) | (N = 1040) | (N = 1088) | (N = 1097) |
| Age (years), | 70.3 ± 9 | 69.2 ± 8.2 | 68.7 ± 9.8 | 68.1 ± 9.5 | 68.9 ± 9.7 | 68.2 ± 9.4 |
| Mean ± SD | | | | | | |
| History of | 80 (78)^{a} | 47 (82)^{b} | 529 (54) | 572 (55) | 609 (56) | 619 (56) |
| CVD, N (%) | | | | | | |
| History of | 36 (35)^{a} | 21 (37)^{b} | 130 (13) | 133 (13) | 166 (15) | 154 (14) |
| HF, N (%) | | | | | | |
| History of | 33 (32)^{a} | 22 (39)^{b} | 185 (19) | 188 (18) | 218 (20) | 210 (19) |
| MI, N (%) | | | | | | |
| History of | 4 (4) | 3 (5) | 46 (5) | 40 (4) | 50 (5) | 43 (4) |
| A-FIB, N (%) | | | | | | |
| Concomitant Med | | | | | | |
| Use, N (%) | | | | | | |
| ACEi/ARB | 35 (34)^{a} | 16 (28)^{b} | 659 (67) | 701 (67) | 694 (64) | 717 (65) |
| Diuretic | 39 (38)^{a} | 15 (26)^{b} | 528 (54) | 586 (56) | 567 (52) | 601 (55) |
| Beta-Blocker | 38 (37)^{a} | 23 (40) | 482 (49) | 506 (49) | 520 (48) | 529 (48) |
| Statin | 57 (55) | 26 (46)^{b} | 640 (65) | 721 (69) | 697 (64) | 747 (68) |
| Calcium | 25 (24)^{a} | 17 (30)^{b} | 406 (41) | 467 (45) | 431 (40) | 484 (44) |
| Channel | | | | | | |
| Blocker | | | | | | |
| eGFR (mL/ | 21.7 ± 4.6 | 22.2 ± 4.7 | 22.5 ± 4.2 | 22.5 ± 4.6 | 22.4 ± 4.3 | 22.5 ± 4.6 |
| min/1.73 m²), | | | | | | |
| Mean ± SD | | | | | | |
| ACR (mg/g), | 353.9^{a} | 302.0 | 199.3 | 216.9 | 210.4 | 220.7 |
| Geo Mean | | | | | | |
| SBP (mmHg), | 139.5 ± 13.3 | 142.3 ± 11.2 | 139.5 ± 11.6 | 139.6 ± 11.8 | 139.5 ± 11.7 | 139.8 ± 11.8 |
| Mean ± SD | | | | | | |
| DBP (mmHg), | 66.4 ± 9.1^{a} | 69.1 ± 8.8 | 70.4 ± 8.7 | 70.8 ± 8.6 | 70.1 ± 8.8 | 70.7 ± 8.7 |
| Mean ± SD | | | | | | |
| BNP (pg/mL) | | | | | | |
| Mean ± SD | 526.0 ± 549.4^{a} | 429.8 ± 434.3^{b} | 223.1 ± 257.5 | 232.3 ± 347.1 | 251.2 ± 309.1 | 242.7 ± 354.7 |
| >100, N (%) | 78 (76)^{a} | 43 (75)^{b} | 547 (56) | 544 (52) | 625 (57) | 587 (54) |
| QTcF (ms) | | | | | | |
| Mean ± SD | 447.9 ± 31.2^{a,c} | 432.5 ± 27.6^{b} | 425.3 ± 27.8 | 424.7 ± 27.9 | 427.4 ± 28.9 | 425.1 ± 28 |
| >450, N (%) | 40 (39)^{a} | 14 (25) | 170 (17) | 167 (16) | 210 (19) | 181 (16) |

^{a}p < 0.05 for BARD patients with HF vs. BARD patients without HF
^{b}p < 0.05 for PBO patients with HF vs. PBO patients without HF
^{c}p < 0.05 for BARD vs. PBO patients with HF

2. Assessment of Clinical Parameters Associated with BNP Increases

As a surrogate of fluid retention, a post-hoc analysis was performed on a subset of patients for whom BNP data were available at baseline and Week 24. Patients in the bardoxolone methyl arm experienced a significantly greater increase in BNP than patients in the placebo arm (Mean±SD: 225±598 vs. 34±209 pg/mL, p<0.01). Also noted was a higher proportion of bardoxolone methyl- vs. placebo-treated patients with increases in BNP at Week 24 (Table 8).

BNP increases at Week 24 did not appear to be related to baseline BNP, baseline eGFR, changes in eGFR, or changes in ACR. However, in bardoxolone methyl-treated patients only, baseline ACR was significantly correlated with Week 24 changes from baseline in BNP, suggesting that the propensity for fluid retention may be associated with baseline severity of renal dysfunction, as defined by albuminuria status, and not with the general changes in renal function, as assessed by eGFR (Table 9).

Further, these data suggest that increases in eGFR, which are glomerular in origin, are distinct anatomically, as sodium and water regulation occurs in the renal tubules.

TABLE 8
Analysis of BNP and eGFR Values of Bardoxolone
Methyl vs. Placebo Patients Stratified by
Changes from Baseline in BNP at Week 24

| | | | Median | Mean | Mean |
|---|---|---|---|---|---|
| WK 24 BNP | Treat- | | BL | BL | WK 24 |
| Change | ment | N | BNP | eGFR | ΔeGFR |
| <25% | PBO | 131 | 119.0 | 23.5 | −0.6 |
| Increase | BARD | 84 | 187.0 | 22.3 | 6.1 |
| 25% to 100% | PBO | 48 | 102.5 | 22.0 | 0.4 |
| Increase | BARD | 45 | 119.0 | 22.7 | 5.5 |
| ≥100% | PBO | 37 | 143.5 | 23.1 | 0.1 |
| Increase | BARD | 82 | 155.0 | 21.9 | 7.6 |

Post-hoc analysis of changes in BNP in BEACON at Week 24.

TABLE 9
Correlations between Changes from Baseline
in BNP at Week 24 and Baseline ACR in Bardoxolone
Methyl vs. Placebo Patients in BEACON

| Treatment | N | Correlation Coefficient | P-value |
|---|---|---|---|
| PBO | 216 | 0.05 | 0.5 |
| BARD | 211 | 0.20 | <0.01 |

Post-hoc analysis of changes in BNP in BEACON at Week 24. Only patients with baseline and Week 24 BNP values included in analysis.

### 3. Serum Electrolytes

No clinically meaningful changes were noted in serum potassium or serum sodium for the subset of patients with 24-hr urine collections (Table 10). The change in serum magnesium levels in bardoxolone methyl-treated patients was consistent with changes observed in prior studies.

TABLE 10
Week 4 Changes from Baseline in Serum Electrolytes in Bardoxolone
Methyl vs. Placebo 24-hour ABPM Sub-Study Patients

| | | Serum | | | Serum | | | Serum | | |
|---|---|---|---|---|---|---|---|---|---|---|
| | | Potassium | | | Sodium | | | Magnesium | | |
| | | (mmol/L) | | | (mmol/L) | | | (mEq/L) | | |
| | | BL | WK4 | WK4 Δ | BL | WK4 | WK4 Δ | BL | WK4 | WK4 Δ |
| PBO | n | 88 | 87 | 87 | 88 | 87 | 87 | 88 | 87 | 87 |
| | Mean ± | 4.8 ± | 4.7 ± | −0.10 ± | 140.2 ± | 139.7 ± | −0.3 ± | 1.72 ± | 1.69 ± | −0.03 ± |
| | SE | 0.1 | 0.1 | 0.04* | 0.2 | 0.3 | 0.2 | 0.03 | 0.03 | 0.02 |
| BARD | n | 83 | 77 | 77 | 83 | 77 | 77 | 83 | 77 | 77 |
| | Mean ± | 4.7 ± | 4.8 ± | 0.10 ± | 140.1 ± | 140.3 ± | 0.2 ± | 1.74 ± | 1.53 ± | −0.21 ± |
| | SE | 0.1 | 0.1 | 0.05*^{†} | 0.3 | 0.3 | 0.3 | 0.02 | 0.03 | 0.02*^{†} |

Data include only BEACON patients enrolled in the 24-hour ABPM sub-study. Changes in serum electrolyte values only calculated for patients with baseline and Week 4 data.
*p < 0.05 for Week 4 versus baseline values within each treatment group;
^{†}p < 0.05 for Week 4 changes in BARD vs. PBO patients.

### 4. 24-Hour Urine Collections

A subset of patients consented to additional 24-hr assessments (sub-study) of ambulatory blood pressure monitoring (ABPM) and 24-hr urine collection at selected visits. Urinary sodium excretion data from BEACON sub-study patients revealed a clinically meaningful reduction in urine volume and excretion of sodium at Week 4 relative to baseline in the bardoxolone methyl-treated patients (Table 11). These decreases were significantly different from Week 4 changes in urine volume and urinary sodium observed in placebo-treated patients. Also of note, reductions in serum magnesium were not associated with renal loss of magnesium.

Additionally, in a pharmacokinetic study in patients with type 2 diabetes and stage 3b/4 CKD administered bardoxolone methyl for eight weeks (402-C-1102), patients with stage 4 CKD had significantly greater reductions of urinary sodium and water excretion than stage 3b CKD patients (Table 12).

TABLE 11
Week 4 Changes from Baseline in 24-hour Urine Volume, Urinary Sodium, and Urinary
Potassium in Bardoxolone Methyl vs. Placebo 24-hour ABPM Sub-Study Patients

| | | Urine | | | Urinary | | | Urinary | | | Urinary | | |
|---|---|---|---|---|---|---|---|---|---|---|---|---|---|
| | | Volume | | | Sodium | | | Potassium | | | Magnesium | | |
| | | (mL) | | | (mmol/24 h) | | | (mmol/24 h) | | | (mmol/24 h) | | |
| | | BL | WK4 | WK4 Δ | BL | WK4 | WK4 Δ | BL | WK4 | WK4 Δ | BL | WK4 | WK4 Δ |
| PBO | n | 87 | 72 | 71 | 81 | 68 | 62 | 81 | 68 | 62 | 59 | 53 | 46 |
| | Mean ± | 2053 ± | 1928 ± | −110 ± | 160 ± | 145 ± | −11 ± | 55 ± | 52 ± | −3 ± | 7.5 ± | 6.0 ± | −0.6 ± |
| | SE | 82 | 89 | 71 | 8 | 8 | 9 | 3 | 3 | 3 | 0.5 | 0.5 | 0.4 |
| BARD | n | 82 | 64 | 63 | 77 | 61 | 57 | 77 | 61 | 57 | 56 | 43 | 40 |
| | Mean ± | 2024 ± | 1792 ± | −247 ± | 164 ± | 140 ± | −27 ± | 60 ± | 52 ± | −7 ± | 7.0 ± | 6.0 ± | −0.9 ± |
| | SE | 83 | 84 | 71* | 9 | 9 | 9* | 3 | 2 | 3* | 0.4 | 0.4 | 0.5 |

Data include only BEACON patients enrolled in the 24-hour ABPM sub-study. Changes at Week 4 only calculated for patients with baseline and Week 4 data.
*p < 0.05 for Week 4 versus baseline values within each treatment group;
† p < 0.05 for Week 4 changes in BARD versus PBO patients.

TABLE 12
Week 8 Changes from Baseline in 24-h Urine Volume and 24-h
Urinary Sodium Bardoxolone Methyl-treated Patients Grouped
by CKD Severity (from a Patient Pharmacokinetic Study)

| | | Urine Volume (mL) | | Urinary Sodium (mmol/24 h) | |
|---|---|---|---|---|---|
| CKD Stage | N | WK 8 Δ | p-value | WK 8 Δ | p-value |
| Stage 3b | 9 | 355 | 0.04 | −12 | 0.02 |
| Stage 4 | 6 | −610 | | −89 | |

Patients were treated with 20 mg bardoxolone methyl once daily for 56 consecutive days; post-treatment follow-up visit occurred on Study Day 84. Data are means. Data include patients with baseline and Week 8 data.

5. Hospital Records from EAC Adjudication Packets

The first scheduled post-baseline assessment in BEACON was at Week 4. Since many of the heart failure events occurred prior to Week 4, the clinical database provides limited information to characterize these patients. Post-hoc review of the EAC case packets for heart failure cases that occurred prior to Week 4 was performed to assess clinical, vitals, laboratory, and imaging data collected at the time of the first heart failure event (Tables 13 and 14).

Examination of these records revealed common reports of rapid weight gain immediately after randomization, dyspnea and orthopnea, peripheral edema, central/pulmonary edema on imaging, elevated blood pressure and heart rate, and preserved ejection fraction. The data suggest that heart failure was caused by rapid fluid retention concurrent with preserved ejection fraction and elevated blood pressure. The preserved ejection fraction is consistent with clinical characteristics of heart failure caused by diastolic dysfunction stemming from ventricular stiffening and impaired diastolic relaxation. This collection of signs and symptoms differs in clinical characteristics from heart failure with reduced ejection fraction, which occurs because of weakened cardiac pump function or contractile impairment (Vasan et al., 1999). Therefore, rapid fluid accumulation in patients with stuff ventricles and minimal renal reserve likely resulted in increased fluid back-up into the lungs and the noted clinical presentation.

Baseline central laboratory values from the clinical database were compared to local laboratory values obtained on admission for heart failure that were included in the EAC packets. Unchanged serum creatinine, sodium, and potassium concentrations in bardoxolone methyl-treated patients with heart failure events that occurred within the first four weeks after randomization (Table 14) suggest that heart failure was not associated with acute renal function decline or acute kidney injury. Overall, the clinical data suggest that the etiology of heart failure is not caused by a direct renal or cardiotoxic effect, but is more likely to be due to sodium and fluid retention.

TABLE 13
Post-Hoc Analysis of Cardiovascular Parameters of Bardoxolone Methyl vs. Placebo
Patients with Heart Failure Events Occurring Within First Four Weeks of Treatment

| | | LVEF | SBP (mmHg) | | | DBP (mmHg) | | | Heart Rate (bpm) | | |
|---|---|---|---|---|---|---|---|---|---|---|---|
| | | HF | BL | HF | Δ | BL | HF | Δ | BL | HF | Δ |
| PBO | n | 4 | 8 | 6 | 6 | 8 | 6 | 6 | 8 | 5 | 5 |
| | Mean ± | 49% ± | 141 ± | 148 ± | 4.7 ± | 65 ± | 65 ± | 1.2 ± | 70 ± | 65 ± | −3.6 ± |
| | SE | 6% | 5 | 11 | 7.2 | 3 | 5 | 3.6 | 3 | 3 | 2.9 |
| BARD | n | 23 | 42 | 33 | 33 | 42 | 34 | 34 | 42 | 32 | 32 |
| | Mean ± | 52% ± | 142 ± | 154 ± | 10.5 ± | 67 ± | 75 ± | 7.9 ± | 67 ± | 81 ± | 14.5 ± |
| | SE | 2% | 2 | 4 | 3.1 | 2 | 2 | 2.1 | 1 | 3 | 2.7 |

Post-hoc analyses of heart failure cases in BEACON. Vital signs at baseline calculated from the average of three standard cuff measurements. Vital signs from HF hospitalization gathered from admission notes included in EAC Adjudication packets and represent singular assessments using different BP monitoring equipment. LVEF only assessed during HF hospitalization. Timing of HF admission calculated from event start date and treatment start date and varied from Weeks 0-4 for each patient.

TABLE 14
Post-Hoc Analysis of Serum Electrolytes of Bardoxolone Methyl vs. Placebo Patients
with Heart Failure Events Occurring Within First Four Weeks of Treatment

| | | Serum | | | Serum | | | Serum | | |
|---|---|---|---|---|---|---|---|---|---|---|
| | | Creatinine | | | Sodium | | | Potassium | | |
| | | (mg/dL) | | | (mmol/L) | | | (mmol/L) | | |
| | | BL | HF | Δ | BL | HF | Δ | BL | HF | Δ |
| PBO | n | 8 | 8 | 8 | 8 | 8 | 8 | 8 | 8 | 8 |
| | Mean ± | 3.4 ± | 3.3 ± | −0.1 ± | 140.0 ± | 137.0 ± | −2.5 ± | 4.5 ± | 4.4 ± | −0.1 ± |
| | SE | 0.2 | 0.2 | 0.2 | 1.0 | 1.0 | 0.6 | 0.2 | 0.1 | 0.2 |
| BARD | n | 42 | 38 | 38 | 42 | 30 | 30 | 42 | 34 | 34 |
| | Mean ± | 2.8 ± | 2.7 ± | −0.1 ± | 140.0 ± | 139.0 ± | −1.0 ± | 4.7 ± | 4.8 ± | 0.1 ± |
| | SE | 0.1 | 0.1 | 0.1 | 0.0 | 1.0 | 0.5 | 0.1 | 0.1 | 0.1 |

Post-hoc analyses of heart failure cases in BEACON. Baseline clinical chemistries assessed at central laboratory. Clinical chemistries from HF hospitalization gathered from hospital notes included in EAC Adjudication packets and represent assessments made at different local laboratories.

### 6. Blood Pressure in BEACON

Mean changes from baseline in systolic and diastolic blood pressures for bardoxolone methyl-treated and placebo-treated patients, based on the average of triplicate standardized blood pressure cuff measurements collected at each visit. Blood pressure was increased in the bardoxolone methyl group relative to the placebo group, with mean increases of 1.9 mmHg in systolic and 1.4 mmHg in diastolic blood pressures noted in the bardoxolone methyl group by Week 4 (the first post-randomization assessment). The increases in systolic blood pressure (SBP) appeared to diminish by Week 32, while diastolic blood pressure (DBP) increases were sustained.

The Week 4 SBP and DBP increases in bardoxolone methyl-treated patients relative to placebo-treated patients were more apparent in the ABPM measurements. This difference in magnitude could be due to the different techniques that were used or to differences in baseline characteristics in the ABPM sub-study patients. Patients in the ABPM sub-study had a higher baseline ACR than the entire population. Regardless, the data demonstrate that bardoxolone methyl increased blood pressure in the BEACON patient population.

### 7. Blood Pressure Changes in Prior CKD Studies

In an open label, dose-ranging study in type 2 diabetic patients with stage 3b-4 CKD (402-C-0902), no dose-related trend in blood pressure changes or change at any individual dose level was noted following 85 consecutive days of treatment at doses ranging from 2.5 to 30 mg of bardoxolone methyl (amorphous dispersion formulation, as used in BEACON). Post-hoc analysis of blood pressure data stratified by CKD stage suggests that bardoxolone methyl-treated patients with stage 4 CKD tended to have increases in blood pressure relative to baseline levels, with the effect most appreciable in the three highest dose groups, whereas bardoxolone methyl-treated patients with stage 3b CKD had no apparent change (Table 15). Although sample sizes in the dose groups stratified by CKD stage are small, these data suggest that the effect of bardoxolone methyl treatment on blood pressure may be related to CKD stage.

Blood pressure values from a phase 2b study with bardoxolone methyl (BEAM, 402-C-0804), which used an earlier crystalline formulation of the drug and employed a titration design, were highly variable and despite noted increases in some bardoxolone methyl treatment groups, no clear dose-related trend was observed in blood pressure.

TABLE 15
Changes from Baseline in Systolic and Diastolic Blood Pressure
in Patients with Type 2 Diabetes and Stage 3b-4 CKD Stratified
by Baseline CKD Stage Dosed with Bardoxolone Methyl

| Dose (mg) | CKD Stage | N | ΔSBP | ΔDBP |
|---|---|---|---|---|
| 2.5 | 3b/4 | 14 | 0.1 ± 4.2 | 0.2 ± 1.8 |
| | 3b | 10 | 0 ± 4.4 | 1 ± 2 |
| | 4 | 4 | 0.3 ± 11 | −1.5 ± 3.9 |
| 5 | 3b/4 | 24 | −1.5 ± 2.3 | −1.4 ± 1.5 |
| | 3b | 19 | −2.1 ± 2 | −1.3 ± 1.4 |
| | 4 | 5 | 0.5 ± 9.1 | −1.4 ± 5.6 |
| 10 | 3b/4 | 24 | −2.4 ± 3.1 | 0.3 ± 1.3 |
| | 3b | 20 | −4.2 ± 3.4 | −0.3 ± 1.3 |
| | 4 | 4 | 6.1 ± 6.7 | 3.6 ± 4.5 |
| 15 | 3b/4 | 48 | 1.1 ± 2.3 | −1 ± 1.2 |
| | 3b | 26 | −2.2 ± 3.3 | −1.3 ± 1.5 |
| | 4 | 22 | 5 ± 2.8 | −0.6 ± 1.9 |
| 30 | 3b/4 | 12 | 7.2 ± 6.2 | 3.2 ± 2.2 |
| | 3b | 3 | −0.4 ± 13.8 | −1.8 ± 3.9 |
| | 4 | 9 | 9.7 ± 7.3 | 4.7 ± 2.5 |

Patients were administered 2.5, 5, 10, 15, or 30 mg doses of bardoxolone methyl once daily for 85 days.

### 8. Blood Pressure and QTcF in Healthy Volunteers

Intensive blood pressure monitoring was employed in a separate Thorough QT Study, which was conducted in healthy volunteers. In both bardoxolone methyl-treated groups, one given the therapeutic dose, 20 mg, which was also studied in BEACON, and one given the supratherapeutic dose of 80 mg, the change in blood pressure did not differ from changes observed in placebo-treated patients after 6 days of once daily administration. Bardoxolone methyl did not increase QTcF as assessed by placebo-corrected QTcF changes (ΔΔQTcF) after 6 days of treatment at 20 or 80 mg.

Bardoxolone methyl has also been tested in non-CKD disease settings. In early clinical studies of bardoxolone methyl in oncology patients (RTA 402-C-0501, RTA 402-C-0702), after 21 consecutive days of treatment at doses that ranged from 5 to 1300 mg/day (crystalline formulation), no mean change in blood pressure was observed across all treatment groups. Similarly, in a randomized, placebo-controlled study in patients with hepatic dysfunction (RTA 402-C-0701), 14 consecutive days of bardoxolone methyl treatment at doses of 5 and 25 mg/day (crystalline formulation) resulted in mean decreases in systolic and diastolic blood pressure (Table 16).

Collectively, these data suggest that bardoxolone methyl does not prolong the QT interval and does not cause blood pressure increases in patients who do not have baseline cardiovascular morbidity or stage 4 CKD.

TABLE 16
Changes from Baseline in Blood Pressure in Patients with
Hepatic Dysfunction Treated with Bardoxolone Methyl

| | | | Mean ΔSBP ± SE (mmHg) | | Mean ΔDBP ± SE (mmHg) | |
|---|---|---|---|---|---|---|
| Dose | | N | D 7 | D 14 | D 7 | D 14 |
| PBO | | 4 | −10 ± 8.5 | −1.3 ± 5.5 | −4.0 ± 2.0 | 0.0 ± 3.1 |
| 5 | mg | 6 | −12.8 ± 5.2 | −8.8 ± 5.1 | −2.0 ± 2.3 | −1.7 ± 3.2 |
| 25 | mg | 6 | −11.5 ± 5.2 | −1.2 ± 3.6 | −4.0 ± 2.8 | −1.5 ± 4.1 |

### 9. Summary and Analysis of Heart Failure

Comparison of baseline characteristics of patients with heart failure events revealed that while the risk for heart failure was higher in the bardoxolone methyl-treated patients, both bardoxolone methyl-treated and placebo-treated patients with heart failure were more likely to have had a prior history of cardiovascular disease and heart failure and on average, had higher baseline ACR, BNP, and QTcF. Thus, development of heart failure in these patients was likely associated with traditional risk factors for heart failure. Additionally, many of the patients with heart failure were in subclinical heart failure prior to randomization, as indicated by their high baseline BNP levels.

As a surrogate of fluid retention after randomization, post-hoc analysis was performed on a subset of patients for whom BNP data were available, and increases were significantly greater in bardoxolone methyl-treated patients vs. placebo-treated patients at Week 24, with the BNP increases in bardoxolone methyl-treated patients directly correlated with baseline ACR. Urinary sodium excretion data from BEACON ABPM sub-study patients revealed a clinically meaningful reduction in urine volume and excretion of sodium at Week 4 relative to baseline in the bardoxolone methyl-treated patients only. In another study, urinary sodium levels and water excretion were reduced in stage 4 CKD patients but not in stage 3b CKD patients. Together, these data suggest that bardoxolone methyl differentially affects sodium and water handling, with retention of these more pronounced in patients with stage 4 CKD.

Consistent with this phenotype for fluid retention, post-hoc review of the narrative descriptions for heart failure events provided in hospital admission notes, together with anecdotal reports from investigators, indicates that heart failure events in bardoxolone methyl-treated patients were often preceded by rapid fluid weight gain and were not associated with acute decompensation of the kidneys or heart.

Blood pressure changes, indicative of overall volume status, were also increased in the bardoxolone methyl group relative to the placebo group as measured by standardized blood pressure cuff monitoring in BEACON. Pre-specified blood pressure analysis in healthy volunteer studies demonstrated no changes in either systolic or diastolic blood pressure. While the intent-to-treat (ITT) analyses of phase 2 CKD studies conducted with bardoxolone methyl showed no clear changes in blood pressure, post-hoc analyses of these studies suggest that increases in both systolic and diastolic blood pressure are dependent on CKD stage. Taken together, these data suggest that the effects of bardoxolone methyl treatment on blood pressure may be associated with CKD disease severity.

Thus, the urinary electrolyte, BNP, and blood pressure data collectively support that bardoxolone methyl treatment can differentially affect volume status, having no clinically detectable effect in healthy volunteers or early-stage CKD patients, while likely promoting fluid retention in patients with more advanced renal dysfunction and with traditional risk factors associated with heart failure at baseline. The increases in eGFR are likely due to glomerular effects whereas effects on sodium and water regulation are tubular in origin. As eGFR change was not correlated with heart failure, the data suggest that effects on eGFR and sodium and water regulation are anatomically and pharmacologically distinct.

The increased risk for heart failure and related adverse events with bardoxolone methyl treatment was not observed in prior studies (Table 17). However, since prior studies of bardoxolone methyl enrolled 10-fold fewer patients, the increased risk, if present, may have been undetectable. Moreover, BEACON limited enrollment to patients with stage 4 CKD, a population known to be at higher risk for cardiovascular events relative to patients with stage 3b CKD. Thus, the advanced nature of renal disease and significant cardiovascular risk burden of the BEACON population (manifested, among other markers, by low baseline eGFR, high baseline ACR, and high baseline BNP levels) were likely important factors in the observed pattern of cardiovascular events.

To examine further the relationship between key endpoints in BEACON and clinically meaningful thresholds of traditional risk factors of fluid overload, an additional post-hoc analysis was performed. Various eligibility criteria related to these risk factors were applied to exclude patients at most risk and explore the resulting outcomes from BEACON. Combinations of select criteria, including exclusion of patients with eGFR of 20 mL/min/1.73 m² or less, markedly elevated levels of proteinuria, and either age over 75 or BNP greater than 200 pg/mL abrogate the observed imbalances (Table 18). Applying these same criteria to SAEs also markedly improves or abrogates the noted imbalances (Table 19). Taken together, these findings suggest utility of these and other renal and cardiovascular risk markers in future selection criteria for clinical studies with bardoxolone methyl.

TABLE 17
Frequency of Treatment-Emergent Adverse Events Related to Heart Failure¹ by Primary System
Organ Class (SOC) Observed in Prior Chronic Kidney Disease Studies with Bardoxolone Methyl

| | | | 0804 (BEAM) | | | | 0902 | | | | |
|---|---|---|---|---|---|---|---|---|---|---|---|
| | | | BARD (Crystalline) | | | | BARD (SDD) | | | | |
| | Study | | PBO | 25 mg | 75 mg | 150 mg | 2.5 mg | 5 mg | 10 mg | 15 mg | 30 mg |
| | SOC | Preferred Term | (N = 57) | (N = 57) | (N = 57) | (N = 56) | (N = 14) | (N = 25) | (N = 28) | (N = 50) | (N = 14) |
| AEs | Metab | Oedema | 3 (5) | 3 (5) | 1 (2) | 3 (5) | 0 | 0 | 0 | 0 | 1 (7) |
| | | peripheral | | | | | | | | | |
| | | Fluid | 0 | 3 (5) | 2 (4) | 0 | — | — | — | — | — |
| | | overload | | | | | | | | | |
| | Genrl | Oedema | 11 (19) | 11 (19) | 10 (18) | 11 (20) | 0 | 3 (12) | 5 (18) | 3 (6) | 3 (21) |
| | | peripheral | | | | | | | | | |
| | | Generalised | 0 | 2 (4) | 0 | 0 | — | — | — | — | — |
| | | oedema | | | | | | | | | |
| | Resp | Dyspnoea | 5 (9) | 2 (4) | 6 (11) | 4 (7) | 0 | 0 | 0 | 0 | 1 (7) |
| | | Dyspnoea | 0 | 1 (2) | 0 | 3 (5) | 1 (7) | 0 | 0 | 0 | 0 |
| | | exertional | | | | | | | | | |
| | | Orthopnoea | 1 (2) | 0 | 0 | 0 | — | — | — | — | — |
| | | Pulmonary | 0 | 0 | 1 (2) | 0 | — | — | — | — | — |
| | | oedema | | | | | | | | | |
| | Inv | Ejection | 0 | 1 (2) | 0 | 0 | — | — | — | — | — |
| | | fraction | | | | | | | | | |
| | | decreased | | | | | | | | | |
| | Card | Oedema | 1 (2) | 4 (7) | 3 (5) | 4 (7) | 0 | 0 | 1 (4) | 1 (2) | 0 |
| | | peripheral | | | | | | | | | |
| | | Cardiac | 3 (5) | 2 (4) | 3 (5) | 3 (5) | 0 | 0 | 1 (4) | 0 | 1 (7) |
| | | failure | | | | | | | | | |
| | | congestive | | | | | | | | | |
| | | Dyspnoea | 0 | 0 | 1 (2) | 0 | — | — | — | — | — |
| | | paroxysmal | | | | | | | | | |
| | | nocturnal | | | | | | | | | |
| SAEs | Card | Cardiac | 3 (5) | 2 (4) | 2 (4) | 2 (4) | 0 | 0 | 1 (4) | 0 | 1 (7) |
| | | failure | | | | | | | | | |
| | | congestive | | | | | | | | | |
| | Genrl | Oedema | 0 | 0 | 0 | 1 (2) | — | — | — | — | — |
| | | peripheral | | | | | | | | | |
| | Metab | Fluid | 0 | 1 (2) | 1 (2) | 0 | — | — | — | — | — |
| | | overload | | | | | | | | | |
| | Resp | Dyspnoea | 1 (2) | 0 | 0 | 0 | — | — | — | — | — |
| | | Pulmonary | 0 | 0 | 1 (2) | 0 | — | — | — | — | — |
| | | oedema | | | | | | | | | |

In 402-C-0804, patients were administered 25, 75, 150 mg of bardoxolone methyl (crystalline formulation) or placebo once daily for 52 weeks.
In RTA402-C-0903, patients were administered 2.5, 5, 10, 15, or 30 mg doses of bardoxolone methyl (SDD formulation) once daily for 85 days.
¹Adverse events with preferred terms matching Standardized MedDRA Queries for cardiac failure outlined in the BEACON EAC Charter (Submission Serial 133, dated Feb. 2, 2012).

TABLE 18
Effect of Excluding Patients with Select Baseline Characteristics on
Primary Endpoints, Heart Failure, and All-Cause Mortality in BEACON

| | | | Eligibility Criteria (N) | | | | | | |
|---|---|---|---|---|---|---|---|---|---|
| | | | | | | | | BL ACR ≤ 1000, | BL ACR ≤ 300, |
| | Observed | | BL | No h/o | BL | BL | | eGFR > 20, | eGFR > 20, |
| Event | | N | BNP ≤ 200 | HF | ACR ≤ 1000 | eGFR > 20 | Age ≤ 75 | Age ≤ 75 | BNP ≤ 200 |
| Heart | BARD | 103 | 22 | 67 | 63 | 56 | 75 | 19 | 5 |
| Failure | PBO | 57 | 16 | 36 | 40 | 37 | 45 | 20 | 3 |
| All-Cause | BARD | 44 | 14 | 35 | 32 | 27 | 20 | 11 | 5 |
| Death | PBO | 31 | 8 | 24 | 21 | 18 | 23 | 11 | 4 |
| ESRD | BARD | 47 | 12 | 35 | 21 | 18 | 38 | 9 | 1 |
| | PBO | 55 | 22 | 44 | 27 | 14 | 46 | 6 | 1 |
| Randomized | BARD | 1088 | 559 | 922 | 798 | 735 | 786 | 368 | 209 |
| Patients | PBO | 1097 | 593 | 943 | 792 | 718 | 829 | 400 | 217 |

Post-hoc analysis of outcomes in BEACON. Observed totals for number of patients with heart failure, all-cause and cardiovascular deaths, and ESRD includes all events through last date of contact (ITT Population).

TABLE 19
Effect of Excluding Patients with Select Baseline Characteristics on Treatment-
Emergent Serious Adverse Events by Primary SOC in BEACON (ITT Population)

| | Primary SOC | | | | | | | | | | | |
|---|---|---|---|---|---|---|---|---|---|---|---|---|
| | | | | | BL ACR ≤ 1000, | | | | BL ACR ≤ 300, | | | |
| | All Patients | | | | eGFR > 20, Age ≤ 75 | | | | eGFR > 20, BNP ≤ 200 | | | |
| | PBO | | BARD | | PBO | | BARD | | PBO | | BARD | |
| Treatment | (N = 1097) | | (N = 1088) | | (N = 400) | | (N = 368) | | (N = 217) | | (N = 209) | |
| Blood and lymphatic | 11 | (1) | 20 | (2) | 3 | (<1) | 4 | (<1) | 2 | (<1) | 0 | |
| system disorders | | | | | | | | | | | | |
| Cardiac disorders | 84 | (8) | 124 | (11) | 32 | (3) | 35 | (3) | 10 | (1) | 16 | (1) |
| Ear and labyrinth disorders | 1 | (<1) | 3 | (<1) | 1 | (<1) | 1 | (<1) | 0 | | 1 | (<1) |
| Endocrine disorders | 1 | (<1) | 1 | (<1) | 1 | (<1) | 1 | (<1) | 1 | (<1) | 1 | (<1) |
| Eye disorders | 2 | (<1) | 3 | (<1) | 1 | (<1) | 1 | (<1) | 1 | (<1) | 0 | |
| Gastrointestinal disorders | 39 | (4) | 46 | (4) | 13 | (1) | 10 | (1) | 8 | (1) | 7 | (1) |
| General disorders and | 20 | (2) | 29 | (3) | 3 | (<1) | 2 | (<1) | 2 | (<1) | 3 | (<1) |
| administration site | | | | | | | | | | | | |
| conditions | | | | | | | | | | | | |
| Hepatobiliary disorders | 8 | (1) | 4 | (<1) | 2 | (<1) | 1 | (<1) | 0 | | 1 | (<1) |
| Immune system disorders | 0 | | 2 | (<1) | 0 | | 0 | | 0 | | 0 | |
| Infections and infestations | 63 | (6) | 79 | (7) | 20 | (2) | 20 | (2) | 12 | (1) | 9 | (1) |
| Injury, poisoning and | 17 | (2) | 19 | (2) | 3 | (<1) | 4 | (<1) | 0 | | 2 | (<1) |
| procedural complications | | | | | | | | | | | | |
| Investigations | 2 | (<1) | 3 | (<1) | 1 | (<1) | 2 | (<1) | 0 | | 0 | |
| Metabolism and | 42 | (4) | 51 | (5) | 11 | (1) | 14 | (1) | 9 | (1) | 5 | (<1) |
| nutrition disorders | | | | | | | | | | | | |
| Musculoskeletal | 13 | (1) | 21 | (2) | 6 | (1) | 9 | (1) | 3 | (<1) | 6 | (1) |
| and connective | | | | | | | | | | | | |
| tissue disorders | | | | | | | | | | | | |
| Neoplasms benign, malignant | 10 | (1) | 11 | (1) | 6 | (1) | 3 | (<1) | 2 | (<1) | 1 | (<1) |
| and unspecified | | | | | | | | | | | | |
| Nervous system disorders | 35 | (3) | 37 | (3) | 13 | (1) | 6 | (1) | 9 | (1) | 4 | (<1) |
| Psychiatric disorders | 3 | (<1) | 3 | (<1) | 1 | (<1) | 2 | (<1) | 1 | (<1) | 1 | (<1) |
| Renal and urinary | 71 | (6) | 52 | (5) | 14 | (1) | 9 | (1) | 2 | (<1) | 4 | (<1) |
| disorders | | | | | | | | | | | | |
| Reproductive system and | 3 | (<1) | 2 | (<1) | 0 | | 0 | | 0 | | 0 | |
| breast disorders | | | | | | | | | | | | |
| Respiratory, thoracic and | 32 | (3) | 43 | (4) | 11 | (1) | 15 | (1) | 7 | (1) | 6 | (1) |
| mediastinal disorders | | | | | | | | | | | | |
| Skin and subcutaneous | 1 | (<1) | 4 | (<1) | 1 | (<1) | 1 | (<1) | 1 | (<1) | 1 | (<1) |
| tissue disorders | | | | | | | | | | | | |
| Surgical and medical | 0 | | 2 | (<1) | 0 | | 1 | (<1) | 0 | | 1 | (<1) |
| procedures | | | | | | | | | | | | |
| Vascular disorders | 18 | (2) | 20 | (2) | 5 | (<1) | 4 | (<1) | 2 | (<1) | 2 | (<1) |

Post-hoc analyses of treatment-emergent serious adverse events in BEACON. Event totals include only SAEs with onset no more than 30 days after a patient's last dose of study drug.

### D. Potential Mechanisms of Fluid Overload in BEACON

Data presented in prior sections suggest that bardoxolone methyl promotes fluid retention in a subset of patients who are at most risk of developing heart failure independent of drug administration. The data suggest that the effects are not associated with acute or chronic renal or cardiac toxicity. Therefore, a comprehensive list of well-established renal mechanisms that affect volume status (Table 20) was explored to determine if any of the etiologies matched the clinical phenotype observed with bardoxolone methyl.

Initial investigation focused on possible activation of the renin-angiotensin-aldosterone system. Activation of this pathway reduces serum potassium due to increased renal excretion. However, bardoxolone methyl did not affect serum potassium and slightly reduced urinary potassium in the BEACON sub-study (Table 10).

Another potential mechanism that was investigated was whether transtubular ion gradient changes may have resulted in sodium and consequent water resorption, since bardoxolone methyl affects serum magnesium and other electrolytes. However, this mechanism also involves potassium regulation, and baseline serum magnesium did not appear to be associated with fluid retention or heart failure hospitalizations.

After other etiologies were excluded for reasons listed in Table 19, suppression of endothelin signaling was the primary remaining potential mechanism of volume regulation that was consistent with the bardoxolone methyl treatment effect in BEACON. Therefore, an extensive investigation of modulation of the endothelin pathway as a potential explanation for fluid retention observed in the BEACON study was conducted.

TABLE 20
Established Renal Mechanisms Affecting Volume Status

| | Na⁺ | K⁺ | Effect on | |
|---|---|---|---|---|
| Mechanism | Retention | Retention | GFR | Comments |
| Bardoxolone Methyl | ↑ | None | ↑ | ↑ Na⁺ retention independent of K⁺in Stage 4 CKD patients, ↑ GFR |
| Endothelin | ↓ | None | ↓ | Suppression of endothelin fits BARD pattern |
| Endothelial Nitric | ↓ | None | ↑ | NO ↓ Na⁺ reabsorption and ↑ GFR |
| Oxide (NO) | | | | BARD ↑ both Na⁺ and GFR |
| | | | | BARD has been shown in vitro and in vivo to increase bioavailable |
| | | | | endothelial NO, but Na⁺ effect is likely independent of NO and GFR |
| | | | | changes |
| Antidiuretic Hormone | ↑ | ↑ | ↓ at ↑ levels | ADH ↑Na⁺ and K⁺ while ↓GFR |
| (ADH) | | | of ADH | BARD does not affect K⁺ and ↑ GFR |
| Transtubular ion | ↑ with | ↑ | No direct | Ion gradients have dual effect on Na+ and K⁺; Cl•HCO₃⁻ gradient often |
| gradients | ↑ GFR | | effect | generated as HCO₃⁻absorption dependent on Na⁺ absorption |
| | | | | BARD does not affect K⁺ or HCO₃⁻ |
| Renin-Angiotensin- | ↑ | ↓ | ↑ | RAAS signaling ↑ K urinary excretion and ↓ serum levels |
| Aldosterone (RAAS) | | | | BARD does not affect K⁺levels and has been shown to ↓ AII levels in |
| | | | | CKD patients and suppress AII signaling in vitro and in vivo |
| Pressure Natriuresis | ↓ | ↓ | Slight ↑ | Volume expansion leads to ↑ medullary plasma flow and ↓ |
| | | | | hypertonicity; ↓ water absorption in the loop of Henle with ↓ of Na⁺ |
| | | | | and K⁺ |
| | | | | BARD-mediated magnitude of volume expansion unlikely sufficient |
| | | | | to promote this effect; BARD ↑ Na⁺ and does not affect K⁺ |
| Prostaglandins (PGE₂, | ↓ | Slight ↓ | ↑ | PGs ↑GFR and ↑Na⁺ urine excretion |
| PGI₂) | | | | BARD ↑ Na⁺ retention, not excretion |
| Natriuretic peptides | ↓ | Slight ↓ | ↑ | Natriuretic peptides have divergent effects on Na⁺ and GFR with |
| | | | | slight effect on K⁺ |
| | | | | BNP and other natriuretic peptides ↑ Na⁺ urine excretion |
| | | | | BARD ↑ Na⁺ retention, not excretion |
| | | | | BARD does no interfere with natriuretic peptides, as GFR would |
| | | | | likely ↓ |
| Peritubular factors | ↑ with | ↑ with | None | Na⁺ and K⁺ move with GFR |
| | ↑ GFR | ↑ GFR | | BARD does not affect K⁺ |

Mechanisms and characteristics of fluid retention.

### 1. Modulation of the Endothelin System

The most directly analogous clinical data for comparison of the effects of known endothelin pathway modulators to the BEACON study are those with the endothelin receptor antagonist (ERA) avosentan. Avosentan was studied in stage 3-4 CKD patients with diabetic nephropathy in the ASCEND study, a large outcomes study to assess the time to first doubling of serum creatinine, ESRD, or death (Mann et al., 2010). While the baseline eGFR in this study was slightly above the mean baseline eGFR in BEACON, patients in the ASCEND study had a mean ACR that was approximately seven-fold higher than BEACON (Table 21). Therefore, the overall cardiovascular risk profile was likely similar between the two studies.

As in BEACON, the ASCEND study was terminated prematurely due to an early imbalance in heart failure hospitalization and fluid overload events. Importantly, avosentan-induced fluid overload-related adverse events, including serious and non-serious, were increased only within the first month of treatment.

Examination of key endpoints in the ASCEND study reveals an approximate three-fold increase in risk of congestive heart failure (CHF) with a modest, non-significant increase in death. Additionally, a small, numerical reduction in ESRD events was also observed. The BEACON study demonstrated similar findings, albeit with a lower incidence of heart failure events. Nonetheless, the two studies showed striking similarities in clinical presentation and timing of heart failure, as well as influences on other key endpoints (Table 22).

TABLE 21
Select Demographic and Baseline Characteristics for Patients in ASCEND* and BEACON (ITT Population)

| | ASCEND | | | BEACON | |
|---|---|---|---|---|---|
| | PBO | Avosentan 25 mg | Avosentan 50 mg | PBO | BARD 20 mg |
| BL Characteristic | (N = 459) | (N = 455) | (N = 478) | (N = 1097) | (N = 1088) |
| Age | 61 ± 9 | 61 ± 9 | 61 ± 9 | 68 ± 9 | 69 ± 10 |
| History of CHF (% of patients) | 13.5% | 14.5% | 14.4% | 15% | 14% |
| Systolic Blood Pressure (mmHg) | 135 ± 15 | 137 ± 14 | 137 ± 14 | 140 ± 12 | 140 ± 12 |
| BMI (kg/m²) | 30 ± 6 | 30 ± 6 | 30 ± 7 | 34 ± 7 | 34 ± 7 |
| eGFR (mL/min/1.73 m²) | 33 ± 11 | 34 ± 11 | 33 ± 11 | 22 ± 5 | 22 ± 4 |
| Median ACR (mg/g) | 1540 | 1416 | 1474 | 221 | 210 |
| ACEi/ARB (% of patients) | 100% | 100% | 100% | 84% | 85% |
| Diuretics (% of patients) | 65% | 64% | 65% | 64% | 64% |

*Results from a randomized, double-blind, placebo-controlled trial of 1392 patients with type 2 diabetes and overt nephropathy receiving avosentan (25 or 50 mg) or placebo in addition to continued angiotensin-converting enzyme inhibition and/or angiotensin receptor blockade (ASCEND).

TABLE 22
Occurrence of Death, End Stage Renal Disease, or Heart
Failure in ASCEND and BEACON (ITT Population)

| | ASCEND | | | BEACON | |
|---|---|---|---|---|---|
| | | Avosentan | Avosentan | | BARD |
| | PBO | 25 mg | 50 mg | PBO | 20 mg |
| Event | (N = 459) | (N = 455) | (N = 478) | (N = 1097) | (N = 1088) |
| CHF | 2.2% | 5.9%* | 6.1%* | 5.0% | 8.8%* |
| Death | 2.6% | 3.6% | 4.6% | 2.8% | 4.0% |
| ESRD | 6.5% | 4.4% | 5.0% | 4.6% | 4.0% |

Occurrence of adjudicated CHF, death, and ESRD events in ASCEND and BEACON. In ASCEND, for an event to be qualified as CHF, the patient had to have typical signs and/or symptoms of heart failure and receive new therapy for CHF and be admitted to the hospital for at least 24 hours; ESRD was defined as need for dialysis or renal transplantation or an eGFR <15 mL/min/1.73 m². Percentages for BEACON include all CHF and ESRD events through last date of contact and total number of deaths at the time of database lock (Mar. 21, 2013). ESRD in BEACON was defined as need for chronic dialysis, renal transplantation, or renal death; additional details and definitions for heart failure are outlined in the BEACON EAC Charter.
*p < 0.05 vs. placebo.

### 2. Mechanism of Endothelin Receptor Antagonist-Induced Fluid Overload

The role of endothelin in fluid overload has been extensively investigated. Through the use of knock-out models in mice, investigators have demonstrated that acute disruption of the endothelin pathway followed by a salt challenge promotes fluid overload. Specific knock-out of endothelin-1 (ET-1), endothelin receptor type A (ETA), endothelin receptor type B (ETB), or the combination of ETA and ETB have all been shown to promote fluid overload in animals with a clinical phenotype consistent with ERA-mediated fluid overload in patients. These effects are caused by acute activation of the epithelial sodium channel (ENaC), which is expressed in the collecting ducts of the kidney, where it reabsorbs sodium and promotes fluid retention (Vachiery and Davenport, 2009).

### 3. Relationship Between Plasma and Urinary Endothelin-1 in Humans

An assessment of plasma and urinary levels of endothelin-1 (ET-1) in humans with eGFR values ranging from stage 5 CKD to supra-normal (8 to 131 mL/min/1.73 m²) has been previously reported (Dhaun et al., 2009). Plasma levels significantly and inversely correlated with eGFR, but due to the modest slope of the curve, meaningful differences of ET-1 were not readily apparent across the large eGFR range assessed. As a surrogate for kidney production of ET-1, the organ where the most ET-1 is produced, fractional excretion of ET-1 was calculated by assessing the plasma and urinary levels of ET-1. From eGFRs>100 to approximately 30 mL/min/1.73 m², urinary levels were relatively unchanged. However, ET-1 levels appear to increase exponentially with decreasing eGFR in patients with stage 4 and 5 CKD. These data suggest that renal ET-1 is primarily dysregulated in patients with advanced (stage 4 and 5) CKD. Based on these published data, the inventors hypothesized that the differential effects on fluid handling by bardoxolone methyl, if due to endothelin modulation, could be due to the disparate endogenous production of ET-1 in the kidney, which is meaningfully increased in stage 4 and 5 CKD patients.

### 4. Bardoxolone Methyl Modulates Endothelin Signaling

As described above, bardoxolone methyl reduces ET-1 expression in human cell lines, including mesangial cells found in the kidney as well as endothelial cell. Furthermore, in vitro and in vivo data suggest that bardoxolone methyl and analogs modulate the endothelin pathway to promote a vasodilatory phenotype by suppressing the vasoconstrictive ETA receptor and restoring normal levels of the vasodilatory ET_{B} receptor. Thus, the potent activation of Nrf2-related genes with bardoxolone methyl is associated with suppression of pathological endothelin signaling and facilitates vasodilation by modulating expression of ET receptors.

### E. Rationale for BEACON Termination

### 1. Adjudicated Heart Failure

Hospitalizations for heart failure or death due to heart failure were among the cardiovascular events adjudicated by the EAC. An imbalance in adjudicated heart failure and related events was the major finding that contributed to the early termination of BEACON. Additionally, heart failure-related AEs, such as edema, contributed to a higher discontinuation rate than expected. The overall imbalance in time-to-first adjudicated heart failure appeared to result from the large contribution of events occurring within the first three to four weeks after initiation of treatment. The Kaplan-Meyer analysis shows that after this initial period the event rates between the treatment arms appear to maintain parallel trajectories. These data suggest an acute, physiologic effect that precipitated hospitalization for heart failure versus a cumulative toxic effect.

### 2. Mortality

At the time of the termination of the study, more deaths had occurred in the bardoxolone methyl group than in the placebo group, and the relationship between mortality and heart failure was unclear. A majority of the fatal outcomes (49 of the 75 deaths) occurring prior to clinical database lock (Mar. 4, 2013) were confirmed as being cardiovascular in nature (29 bardoxolone methyl patients vs. 20 placebo patients). Most of the cardiovascular deaths were classified as “cardiac death—not otherwise specified,” based on pre-specified definitions outlined in the BEACON EAC charter. On final analysis, the Kaplan-Meier analysis for overall survival showed no apparent separation until approximately Week 24. There were three fatal heart failure events, all in bardoxolone methyl-treated patients. In addition, as reflected in Table 19, the percentage of deaths occurring in patients that were over 75 years old was higher in bardoxolone methyl-treated patients compared to placebo-treated patients. Notably, if patients over 75 years old are excluded, the numbers of fatal events in the bardoxolone methyl arm compared to the placebo arm are 20 and 23, respectively.

3. Summary of Other Safety Data from BEACON

In addition to the effects of bardoxolone methyl treatment on eGFR and renal SAEs, the number of hepatobiliary SAEs was reduced in the bardoxolone methyl group relative to the placebo group (4 versus 8, respectively; Table 5), and no Hy's Law cases were observed. The number of neoplasm-related SAEs was also balanced across both groups. Lastly, bardoxolone methyl treatment was not associated with QTc prolongation, as assessed by ECG assessments at Week 24 (Table 23).

TABLE 23
Change from Baseline in QTcF at Week 24 in Bardoxolone Methyl
versus Placebo Patients in BEACON (Safety Population)

| | Observed | | Change from baseline | |
|---|---|---|---|---|
| Timepoint/ | | Bardoxolone | | Bardoxolone |
| QTcF interval | Placebo | methyl | Placebo | methyl |
| (msec) | N = 1093 | N = 1092 | N = 1093 | N = 1092 |
| n | 719 | 639 | 719 | 637 |
| Mean (SD) | 428.8 (29.2) | 425.8 (26.5) | 3.6 (16.4) | −0.9 (19.2) |
| Range (min, max) | 362, 559 | 355, 518 | −59, 82 | −88, 69 |
| Quartiles (25th, | 408, 426, | 407, 425, | −7, 3, | −13, −1, |
| median, 75th) | 445 | 443 | 13 | 10 |

Data includes only ECG assessments collected on or before a patient's last dose of study drug. Visits are derived relative to a patient's first dose of study drug.

### F. BEACON Conclusions

In summary, interrogation of data from studies conducted with bardoxolone methyl revealed that the drug can differentially regulate fluid retention, with no clinically detectable effect in healthy volunteers or early-stage CKD patients, while likely pharmacologically promoting fluid retention in patients with advanced renal dysfunction. Since the development of heart failure in both bardoxolone methyl- and placebo-treated patients was associated with traditional risk factors for heart failure, this pharmacological effect in patients with baseline cardiac dysfunction may explain the increased risk for heart failure with bardoxolone methyl treatment in BEACON. These data suggest that decreasing the overall risk for heart failure in future clinical studies by selecting a patient population with lower baseline risk for heart failure should avoid increases in heart failure associated with bardoxolone methyl treatment. Importantly, the available data show that fluid overload in BEACON was not caused by a direct renal or cardiac toxicity. The clinical phenotype of fluid overload is similar to that observed with ERAs in advanced CKD patients, and preclinical data demonstrate that bardoxolone methyl modulates the endothelin pathway. As acute disruption of the endothelin pathway in advanced CKD patients is known to activate a specific sodium channel (ENaC) that can promote acute sodium and volume retention (Schneider, 2007), these mechanistic data, along with the clinical profile of bardoxolone methyl patients with heart failure, provide a reasonable hypothesis to the mechanism of fluid retention in BEACON. Because compromised renal function may be an important factor that contributes to a patient's inability to compensate for short-term fluid overload, and because relatively limited numbers of patients with earlier stages of CKD have been treated to date, exclusion of patients with CKD (e.g., patients with an eGFR<60) from treatment with BARD and other AIMs may be prudent and may be included as an element of the present invention.

### VIII. Diagnostic Tests

### A. Measurement of B-Type Natriuretic Peptide (BNP) Levels

B-type natriuretic peptide (BNP) is a 32-amino acid neurohormone that is synthesized in the ventricular myocardium and released into circulation in response to ventricular dilation and pressure overload. The functions of BNP include natriuresis, vasodilation, inhibition of the renin-angiotensin-aldosterone axis, and inhibition of sympathetic nerve activity. The plasma concentration of BNP is elevated among patients with congestive heart failure (CHF), and increases in proportion to the degree of left ventricular dysfunction and the severity of CHF symptoms.

Numerous methods and devices are well known to the skilled artisan for measuring BNP levels in patient samples, including serum and plasma. With regard to polypeptides, such as BNP, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,170 and 5,955,377. In a specific example, B-type natriuretic peptide (BNP) levels may be determined by the following method(s): protein immunoassays as described in US Patent Publication 2011/0201130, which is incorporated by reference in its entirety herein. Furthermore, a number of commercially available methods exist (e.g., Rawlins et al., 2005, which is incorporated herein by reference in its entirety).

### B. Measurement of Albumin/Creatinine Ratio (ACR)

Conventionally, proteinuria is diagnosed by a simple dipstick test. Traditionally, dipstick protein tests are quantified by measuring the total quantity of protein in a 24-hour urine collection test.

Alternatively the concentration of protein in the urine may be compared to the creatinine level in a spot urine sample. This is termed the protein/creatinine ratio (PCR). The UK Chronic Kidney Disease Guidelines (2005; which are incorporated herein by reference in their entirety) states PCR is a better test than 24-hour urinary protein measurement. Proteinuria is defined as a protein/creatinine ratio greater than 45 mg/mmol (which is equivalent to albumin/creatinine ratio of greater than 30 mg/mmol or approximately 300 mg/g as defined by dipstick proteinuria of 3+) with very high levels of proteinuria being for a PCR greater than 100 mg/mmol.

Protein dipstick measurements should not be confused with the amount of protein detected on a test for microalbuminuria, which denotes values for protein for urine in mg/day versus urine protein dipstick values which denote values for protein in mg/dL. That is, there is a basal level of proteinuria that can occur below 30 mg/day which is considered non-pathological. Values between 30-300 mg/day are termed microalbuminuria which is considered pathologic. Urine protein lab values for microalbumin of >30 mg/day correspond to a detection level within the “trace” to “1+” range of a urine dipstick protein assay. Therefore, positive indication of any protein detected on a urine dipstick assay obviates any need to perform a urine microalbumin test as the upper limit for microalbuminuria has already been exceeded.

C. Measurement of Estimated Glomerular Filtration Rate (eGFR)

A number of formulae have been devised to estimate GFR values on the basis of serum creatinine levels. A commonly used surrogate marker for estimate of creatinine clearance (eC_{Cr}) is the Cockcroft-Gault (CG) formula, which in turn estimates GFR in mL/min. It employs serum creatinine measurements and a patient's weight to predict the creatinine clearance. The formula, as originally published, is:

$$eCCr = \frac{( 140 - Age ) \times Mass ( in kg )}{72 \times Serum creatinine ( in \frac{mg}{dL} )}$$

This formula expects weight to be measured in kilograms and creatinine to be measured in mg/dL, as is standard in the USA. The resulting value is multiplied by a constant of 0.85 if the patient is female. This formula is useful because the calculations are simple and can often be performed without the aid of a calculator.

When serum creatinine is measured in μmol/L, then:

$$eCCr = \frac{( 140 - Age ) \times Mass ( in kg ) \times Constant}{Serum creatinine ( in \frac{µmol}{L} )}$$

where Constant is 1.23 for men and 1.04 for women.

One interesting feature of the Cockcroft and Gault equation is that it shows how dependent the estimation of C_{Cr} is based on age. The age term is (140−age). This means that a 20-year-old person (140−20=120) will have twice the creatinine clearance as an 80-year-old (140−80=60) for the same level of serum creatinine. The CG equation assumes that a woman will have a 15% lower creatinine clearance than a man at the same level of serum creatinine.

Alternatively, eGFR values may be calculated using the Modification of Diet in Renal Disease (MDRD) formula. The 4-variable formula is as follows:

$$eGFR = 175 \times Standardized serum {creatinine}^{- 1.154} \times {Age}^{- 0.203} \times C$$

where C is 1.212 if the patient is a black male, 0.899 if the patient is a black female, and 0.742 if the patient is a non-black female. Serum creatinine values are based on the IDMS-traceable creatinine determination (see below).

Chronic kidney disease is defined as a GFR less than 60 mL/min/1.73 m² that is present for three or more months.

### D. Measurement of Serum Creatinine Levels

A serum creatinine test measures the level of creatinine in the blood and provides an estimate glomerular filtration rate. Serum creatinine values in the BEACON and BEAM trials were based on the isotope dilution mass spectrometry (IDMS)-traceable creatinine determinations. Other commonly used creatinine assay methodologies include (1) alkaline picrate methods (e.g., Jaffe method [classic] and compensated [modified] Jaffe methods), (2) enzymatic methods, (3) high-performance liquid chromatography, (4) gas chromatography, and (5) liquid chromatography. The IDMS method is widely considered to be the most accurate assay (Peake and Whiting, 2006, which is incorporated herein by reference in its entirety).

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Effect of CYP3A4 Inhibitors on Bardoxolone Methyl Pharmacokinetics

Bardoxolone methyl and its analogs are oleanolic acid-derived synthetic triterpenoid compounds that potently induce the nuclear factor erythroid 2-related factor (Nrf2)-Kelch-like ECH-associated protein 1 (Keap1) pathway (Wu, 2011; Rojas-Rivera, 2012). Through interaction with the Nrf2 repressor molecule, Keap1, bardoxolone methyl and its analogs promote translocation of Nrf2 to the nucleus, where Nrf2 binds to antioxidant response elements in the promoter region of its target genes, leading to induction of many antioxidant and cytoprotective enzymes and related proteins (Lee, 2009; Dinkova-Kostova, 2005). Bardoxolone methyl and its analogs are also potent inhibitors of the nuclear factor-κB (NF-κB) inflammatory pathway through both direct (i.e., inhibition of inhibitor of NF-κB kinase subunit β kinase activity) and indirect mechanisms (i.e., detoxification of reactive oxygen species) (Osburn, 2008). Because of this dual mechanism of action, bardoxolone methyl and its analogs are hypothesized to have potential therapeutic relevance in a variety of disease settings involving oxidative stress and inflammation.

At the time of this study, approximately 1,950 individuals had been exposed to bardoxolone methyl in prior clinical studies. Sixteen studies were completed (7 in patients with chronic kidney disease [CKD] who also had type 2 diabetes, 4 in non-CKD indications, and 5 in healthy subjects), and 4 studies were ongoing at the time this study was conducted (3 in patients with pulmonary hypertension and 1 in patients with Alport syndrome).

The clinical programs conducted to date have demonstrated that bardoxolone methyl is generally well tolerated. Adverse events of special interest that were reported in previous clinical studies included fluid overload, transaminase and gamma-glutamyl transpeptidase elevations, muscle spasms, weight loss, and hypomagnesemia.

The clinical PK of bardoxolone methyl was previously evaluated in healthy volunteers and patients with cancer, CKD, pulmonary arterial hypertension, and hepatic dysfunction. When administered orally, peak plasma concentrations of bardoxolone methyl were typically observed between 1 and 6 hours after dose administration. Systemic exposure to bardoxolone methyl increased linearly with increasing dose from 20 to 60 mg; however, little to no increase in systemic exposure was observed when the dose was increased from 60 to 80 mg. While there was no appreciable food effect on the total systemic exposure of bardoxolone methyl, administration with food resulted in approximately 71% higher peak plasma levels (maximum observed plasma concentration).

Following oral administration of bardoxolone methyl, excretion occurs primarily as inactive metabolites in the feces, and to a much smaller extent, in the urine. Following a single oral dose, bardoxolone methyl is eliminated with a mean half-life of approximately 48 to 60 hours.

This study was designed to evaluate the effect of a strong CYP3A inhibitor (itraconazole) on the PK of bardoxolone methyl. In vitro data from experiments with human liver microsomes and recombinantly-expressed CYPs have shown that bardoxolone methyl is metabolized by CYP3A4. In human liver microsomes, disappearance of bardoxolone methyl is inhibited by the CYP3A4/5 inhibitor ketoconazole (1 μM) up to 77%, with Kₘ and Vmax values 1.5 μM and 1210 pmol/mg/min, respectively. Further, the kinetic constants for bardoxolone methyl in recombinant CYP3A4 (rCYP3A4) are summarized in the table below:

Enzyme Kinetic Constants in rCYP3A4 for Bardoxolone Methyl

| | Kₘ | Vₘₐₓ | CLᵢₙₜ |
|---|---|---|---|
| Compound | (μM) | (pmol/min/pmol CYP) | (μL/min/pmol CYP) |
| Bardoxolone methyl | 1.72 | 30.1 | 17.5 |

A reduction in the functional capacity of CYP3A4 enzymes might impact the systemic exposure to bardoxolone methyl. Itraconazole is a potent inhibitor of CYP3A4, and is, therefore, a good probe to study the potential impact of CYP3A4 inhibition (Liu, 2016; Ke, 2014). This study's primary objective was to evaluate the potential effects of itraconazole on the PK of bardoxolone methyl. This study's secondary objective was to assess the safety of concomitant administration of bardoxolone methyl with a strong CYP3A inhibitor (itraconazole).

Itraconazole is an azole antifungal agent, marketed under the brand name SPORANOX®. Following oral administration, peak plasma concentrations of itraconazole are achieved between 2 and 5 hours. Following repeated administration of itraconazole, accumulation is observed with peak steady state achieved in about 15 days, with peak plasma levels of 0.5 μg/mL, 1.1 μg/mL, and 2.0 μg/mL after administration of 100 mg once daily, 200 mg once daily, and 200 mg twice daily, respectively.

Itraconazole undergoes extensive hepatic metabolism with a reported terminal half-life of between 16 and 28 hours following a single administration and between 34 and 42 hours upon multiple administrations. Itraconazole excretion primarily occurs via inactive metabolites in feces and urine. Renal excretion of itraconazole and the active metabolite hydroxy-itraconazole accounts for less than 1% of an intravenous dose.

Co-administration of itraconazole tablets and drugs primarily metabolized by CYP3A4 will likely result in increased plasma concentrations of the concomitant drugs that could affect both therapeutic and adverse effects. Additional information on itraconazole can be found in the full prescribing information for SPORANOX® (Janssen Pharmaceuticals, Inc., 2017).

Methodology: This single-center, open-label, fixed-sequence crossover study was conducted in 16 healthy volunteers. Subjects received a single administration of bardoxolone methyl (10 mg) alone in Period 1 (Study Day 1). In Period 2, the same subjects received a daily dose of 200 mg of itraconazole from Study Day 15 to Study Day 27, with a single administration of bardoxolone methyl (10 mg) on Study Day 18. A detailed schedule of assessments is shown in Table 24.

TABLE 24
Schedule of Assessments

| | Study Day | | | | | | | | | | | |
|---|---|---|---|---|---|---|---|---|---|---|---|---|
| | Screening | | | Period 1 | | | Washout | | | Period 2 | | |
| Assessment | −28 to −2 | −1 | 1 | 2-8 | 9 | 10-13 | 14 | 15 | 16-17 | 18 | 19-27 | 28/ET |
| Informed consent^{a} | X | | | | | | | | | | | |
| Inclusion/exclusion | X | X | | | | | | | | | | |
| Demographics | X | X | | | | | | | | | | |
| Concomitant medicants | X | -------------------X------------------- | | | | | ------------------------X----------------------- | | | | | X |
| Medical history | X | X | | | | | | | | | | |
| Height and weight^{b} | X | X | | | | | X | | | | | |
| Physical exam | X | X | | | | | X | | | | | |
| 12-lead ECG^{c} | X | X | X | X | | | X | | | X | X | X |
| Vital signs^{d} | X | X | X | X | X | | X | X | X | X | X | X |
| Pregnancy test | X | X | | | | | X | | | | | X |
| (females only) | | | | | | | | | | | | |
| Clinical | X | X^{e} | | | | | X | | | | | X |
| laboratory tests | | | | | | | | | | | | |
| Drug and alcohol | X | X | | | | | X | | | | | |
| screen^{f} | | | | | | | | | | | | |
| Confinement^{g} | | -------------------X------------------- | | | | | ------------------------X----------------------- | | | | | X |
| Administer | | | X | | | | | | | X | | |
| bardoxolone | | | | | | | | | | | | |
| methyl^{h} | | | | | | | | | | | | |
| Administer | | | | | | | | --------------------X----------------- | | | | |
| intraconzole | | | | | | | | | | | | |
| Plasma PK | | | X | X | X | | | | | X | X | X |
| samplesⁱ | | | | | | | | | | | | |
| Adverse event | X | X | X | X | X^{k} | | X | X | X | X | X | X |
| collection^{j} | | | | | | | | | | | | |

^{a}Performed within 28 days prior to initial study drug administration on Study Day 1.
^{b}Height and BMI were collected at screening only; weight was collected at all other days as indicated.
^{c}The 12-lead ECGs were obtained at screening, on Study Day −1, on Study Day 1 prior to dosing and 3 and 24 hours after dosing; and on Study Day 14 and on Study Day 18 prior to dosing and 3 and 24 hours after dosing. The 12-lead ECGs were obtained in triplicate.
^{d}Blood pressure, heart rate, and oral temperature assessments were conducted at screening and daily on Study Day −1 through Study Day 9 and daily on Study Day 14 through Study Day 28. On Study Day 1 and Study Day 18, assessments were conducted pre-dose, then 1, 2, 4, 6, 8, and 12 hours after bardoxolone methyl administration.
^{e}The administrative memorandum dated 2 Oct. 2017 clarified that dosing was able to proceed on Study Day 1 if the BNP laboratory results from Study Day −1 were pending, provided the BNP was <200 pg/mL at screening.
^{f}The drug and alcohol screen was not to be completed on Study Day 28/ET.
^{g}Confinement ended after the last PK sample collection and exit procedures were completed.
^{h}Bardoxolone methyl was administered under fasted conditions. On Study Day 18, bardoxolone methyl was administered approximately 1 hour after administration of itraconazole.
ⁱBlood samples for PK were collected on Study Day 1 at pre-dose and 0.5, 1, 2, 3, 4, 5, 6, 8, 10, 12, 18, 24, 36, 48, 72, 96, 120, 144, 168, and 192 hours after dosing. Blood samples for bardoxolone methyl were collected on Study Day 18 at pre-dose and 0.5, 1, 2, 3, 4, 5, 6, 8, 10, 12, 18, 24, 36, 48, 72, 96, 120, 144, 168, 192, 216, and 240 hours after dosing.
BMI = body mass index; BNP = B-type natriuretic peptide; ECG = electrocardiogram; ET = early termination; exam = examination; PK = pharmacokinetic(s).

There was a screening period between Study Day −28 and Study Day −1. Subjects were confined at the clinic during Period 1 from Study Day −1 through Study Day 9. For Period 2, subjects returned to the clinic on Study Day 14 and were confined through Study Day 28. Pharmacokinetic samples were collected at multiple times during the confinement periods.

Period 1 (Study Day −1 to Study Day 9): Upon meeting eligibility criteria, 16 subjects were enrolled in the study and commenced Period 1. During this period, all subjects received a single oral dose of 10 mg bardoxolone methyl on Study Day 1 under fasted conditions. The study drug (bardoxolone methyl) was taken with approximately 240 mL of water following a minimum of a 10-hour fast.

Period 2 (Study Day 14 to Study Day 28): The same subjects from Period 1 returned after a washout period (Study Day 9 through Study Day 13) for Period 2. During Period 2, the subjects received daily oral administration of 200 mg itraconazole from Study Day 15 to Study Day 27 with approximately 240 mL of water. Itraconazole was administered under fed conditions on all dosing days except Study Day 18, when it was dosed under fasted conditions. On Study Day 18, subjects also received a single oral administration of 10 mg bardoxolone methyl under fasted conditions. Bardoxolone methyl was administered 1 hour after the 200 mg itraconazole dose on Study Day 18. The study drug (bardoxolone methyl) was taken with approximately 240 mL of water following a minimum of a 10-hour fast.

Number of subjects (planned and analyzed): Approximately 16 healthy volunteers were planned to be enrolled in this study. The number of subjects enrolled and analyzed for the study was 16. Table 25 summarizes demographic and baseline characteristics of the subjects. One subject withdrew early from the study due to a family emergency on Study Day 5.

TABLE 25
Demographic and Baseline Characteristics

| | Period 1 | Period 2 | |
|---|---|---|---|
| | Bardoxolone | | Bardoxolone Methyl + |
| Characteristic | Methyl | Itraconazole | Itraconazole |
| Statistic/Category | (N = 16) | (N = 15) | (N = 15) |

Age at informed consent (year)

| n | 16 | | 15 | | 15 | |
|---|---|---|---|---|---|---|
| Mean (SD) | 39.1 | (7.65) | 39.4 | (7.79) | 39.4 | (7.79) |

Sex - n (%)

| | | | | | | |
|---|---|---|---|---|---|---|
| Male | 13 | (81.3) | 12 | (80.0) | 12 | (80.0) |
| Female | 3 | (18.8) | 3 | (20.0) | 3 | (20.0) |

Race (n) (%)

| | | | | | | |
|---|---|---|---|---|---|---|
| Black or African | 14 | (87.5) | 13 | (86.7) | 13 | (86.7) |
| American | | | | | | |
| White | 2 | (12.5) | 2 | (13.3) | 2 | (13.3) |

Ethnicity - n (%)

| | | | | | | |
|---|---|---|---|---|---|---|
| Not Hispanic or | 16 | (100.0) | 15 | (100.0) | 15 | (100.0) |
| Latino | | | | | | |
| Hispanic or | 0 | (0.0) | 0 | (0.0) | 0 | (0.0) |
| Latino | | | | | | |

Height at screening (cm)

| n | 16 | | 15 | | 15 | |
|---|---|---|---|---|---|---|
| Mean (SD) | 179.92 | (9.137) | 179.87 | (9.456) | 179.87 | (9.456) |

Body weight at screening (kg)

| n | 16 | | 15 | | 15 | |
|---|---|---|---|---|---|---|
| Mean (SD) | 85.92 | (12.469) | 85.36 | (12.698) | 85.36 | (12.698) |

Baseline BMI (kg/m²)^{a}

| n | 16 | | 15 | | 15 | |
|---|---|---|---|---|---|---|
| Mean (SD) | 26.49 | (2.822) | 26.33 | (2.846) | 26.33 | (2.846) |

Note:
Period 1 was a single dose of bardoxolone methyl. Period 2 was a single dose of bardoxolone methyl and daily doses of itraconazole.
^{a}Baseline was defined as the measurement at screening.
BMI = body mass index; SD = standard deviation.

Criteria for inclusion: The study population included generally healthy males and females between 18 and 55 years of age, inclusive. General good health was based upon the results of medical history, physical examination, vital signs, laboratory profile, and a 12-lead electrocardiogram (ECG), as judged by the investigator. Females must not have been planning a pregnancy, pregnant, or lactating. Female subjects of childbearing potential must have had a negative serum pregnancy test result before enrollment into the study and must have been willing to use contraception or abstain from sexual activity for the duration of the study. Male subjects must have been surgically sterile or practicing contraception, from initial study drug administration through 90 days after administration of the last dose of study drug. Male subjects must have agreed to abstain from sperm donation through 90 days after administration of the last dose of study drug. Subjects must have had a body mass index greater than or equal to 18 to less than or equal to 31 kg/m², inclusive.

Criteria for exclusion: Subjects with a history of clinically significant drug allergies, including allergies to any of the components of the investigational product and/or clinically significant food allergies, as determined by the investigator, were excluded. Subjects with the presence or history of any significant cardiovascular, gastrointestinal, hepatic, renal, pulmonary, hematologic, endocrine, immunologic, dermatologic, neurologic, or psychiatric disease, as determined by the investigator, were excluded. Subjects with the presence of any other condition (including surgery) known to interfere with the absorption, distribution, metabolism, or excretion of medicines were excluded. Subjects with a known hypersensitivity to any component in the formulations of bardoxolone methyl or SPORANOX® were excluded. Patient with a recent (6 month) history of drug or alcohol abuse were excluded. Subjects with B-type natriuretic peptide level >200 pg/mL at screening, alanine transaminase or aspartate aminotransferase level above the upper limit of normal at screening, history of clinically significant left-sided heart disease and/or clinically significant cardiac disease, or clinically significant abnormal ECG (ECG with QT interval corrected for heart rate using Fridericia's correction >450 msec was exclusionary) at screening were excluded from the study. Subjects with a positive test result for hepatitis B surface antigen (HBsAg), hepatitis C virus antibody (HCV Ab), or human immunodeficiency virus antibodies (HIV Abs) at screening were excluded. Subjects who had used tobacco or nicotine-containing products within the 6-month period preceding study drug administration were excluded. Subjects were not allowed to consume alcohol, grapefruit, grapefruit products, star fruit, star fruit products, or Seville oranges within the 72-hour period prior to study drug administration. Subjects who used any medications (over-the-counter and/or prescription medication), vitamins, and/or herbal supplements within the 30-day period prior to study drug administration or within 5 half-lives (if known), whichever was longer, and those who required medications (over-the-counter and/or prescription medication), vitamins, and/or herbal supplements on a regular basis were excluded from the study.

Test product, dose and mode of administration, batch number: Bardoxolone methyl (10-mg capsules) was supplied to the study site for oral administration. Lot number 60707-1 (manufacturing batch number 3156042R; packaging batch number 3156562) of bardoxolone methyl was used in this study. A 10-mg dose of bardoxolone methyl was selected for this study because it is a clinically relevant dose that is within the range of doses that produce reasonably linear PK in humans. Specifically, the 10-mg dose is within the range of doses (from 2.5 to 30 mg) that were being evaluated in Phase 2 and Phase 3 studies that were ongoing at the time of this study. Bardoxolone methyl (10-mg capsules) was administered as 1 capsule (10 mg) orally in the morning of Study Day 1 and Study Day 18 under fasted conditions by study personnel. Bardoxolone methyl was administered 1 hour after itraconazole administration on Study Day 18.

Itraconazole dose and mode of administration, batch number: Commercially available itraconazole (SPORANOX®, 100-mg capsules) with standard approved labeling was supplied by the study site for oral administration. Lot number 17BG306X of SPORANOX® was used in this study. The itraconazole dose and dose regimen was selected to achieve and sustain adequate CYP3A4 inhibition prior to and following administration of bardoxolone methyl. Itraconazole (SPORANOX®, 100-mg capsules) was administered as 2 capsules (200 mg) orally in the morning of Study Day 15 through Study Day 27 (13 days) by study personnel. A meal was administered immediately prior to dosing on all days except on Study Day 18, when the administration of itraconazole was performed under fasted conditions.

Duration of treatment: Period 1: Subjects received a single 10-mg dose of bardoxolone methyl on Study Day 1. Period 2: Subjects received a daily 200-mg dose of itraconazole from Study Day 15 to Study Day 27 (13 days). Subjects also received a single 10-mg dose of bardoxolone methyl on Study Day 18.

Meals and dietary requirements: Subjects received a standardized diet for all meals during confinement. During confinement, subjects consumed only the scheduled meals provided in the study and water to quench thirst. The subjects abstained from all other food and beverages. No food or beverage, except for water to quench thirst, was allowed from 10 hours prior to dosing until after the collection of the 4-hour blood samples on Study Day 1 and Study Day 18. No fluids except those required for dose administration were allowed for 1 hour before dosing and 1 hour after dosing. On Study Day 1 and Study Day 18, subjects were not to be served breakfast. Lunch was served approximately 4 hours after bardoxolone methyl dosing, dinner at approximately 6 hours after lunch, and a snack at approximately 3 hours after dinner. On Study Day 2 through Study Day 8, breakfast was served following collection of the 24-, 48-, 72-, 96-, 120-, 144-, and 168-hour blood samples, with lunch at approximately 4 hours after breakfast, dinner at approximately 6 hours after lunch, and a snack at approximately 3 hours after dinner. On Study Day 15 through Study Day 17 and Study Day 19 through Study Day 27, breakfast was served immediately prior to itraconazole administration and approximately 1 hour prior to the collection of the 24-, 48-, 72-, 96-, 120-, 144-, 168-, 192-, and 216-hour blood samples, with lunch at approximately 4 hours after breakfast, dinner at approximately 6 hours after lunch, and a snack at approximately 3 hours after dinner. On Study Day 9, breakfast (optional) was served following collection of the 192-hour blood sample and completion of the scheduled study procedures. The meal content was identical on the intensive PK sampling days (Study Day 1 and Study Day 18). A dietician determined the composition (protein, fat, carbohydrate, and total calories) of these meals and a record was kept with the source documents. The sequence of starting meals on the dosing days was maintained such that the time intervals between dosing and meals were essentially the same for all subjects.

Statistical methods: The PK Concentration Population included all subjects with no major deviations to study treatment intake, who had at least 1 measurable plasma concentration. The PK Parameter Population included all subjects with no major deviations to study treatment intake, who had sufficient plasma concentration data to characterize the maximum plasma concentration observed directly from data (Cₘₐₓ) and area under the plasma concentration versus time curve (AUC).

Standard non-compartmental analysis methods were used for the calculation of PK parameters. The actual collection time was used for evaluation of PK data. The Linear Up Log Down method (equivalent to the Linear Up/Log Down option in WinNonlin® Professional) was used in the computation of AUC estimates.

The Cmax and AUC estimates (AUC from time 0 to the last quantifiable concentration [AUC0-t] and AUC from time 0 extrapolated to infinity time, calculated as AUC0-t+the last quantifiable concentration/λz, where λz was the first-order elimination rate constant calculated from the slope of logarithmic-linear regression of the terminal phase [AUC0-∞]) of bardoxolone methyl when administered alone or in the presence of itraconazole were compared using a paired t-test for the natural logarithm-transformed Cmax and AUC values. Two-sided 90% confidence intervals (CIs) for geometric mean (GM) ratios of PK parameters (Period 2 versus Period 1) were calculated. A subject must have had a calculable PK parameter in both periods in order to be included in the analysis for that parameter. The PK parameter analyses were based on the PK Parameter Population.

Pharmacokinetics evaluation and results: Blood samples were collected in this study to characterize the PK of bardoxolone methyl in the presence and absence of itraconazole in healthy volunteers. Blood samples for analysis of bardoxolone methyl in plasma were collected by venipuncture before dosing (0 hour), and at 0.5, 1, 2, 3, 4, 5, 6, 8, 10, 12, 18, 24, 36, 48, 72, 96, 120, 144, 168, 192, 216, and 240 hours (216 and 240 hours in Period 2 only) following bardoxolone methyl administration.

The PK variables listed below were calculated from the individual plasma bardoxolone methyl concentration versus time profiles:
Cₘₐₓ: Maximum plasma concentration observed directly from data;
tₘₐₓ: Time to reach Cₘₐₓ directly from data;
λ_{z}: First-order elimination rate constant calculated from the slope of logarithmic-linear regression of the terminal phase;
t_{1/2}: Terminal elimination half-life, calculated as ln(2)/λ_{z};
AUC₀₋ₜ: AUC from time 0 to the last quantifiable concentration (Cₗₐₛₜ);
AUC_{0-∞}: AUC from time 0 extrapolated to infinity time, calculated as AUC₀₋ₜ+Cₗₐₛₜ/λ_{z};
AUC extra %: Percent extrapolated AUC calculated as 100*(AUC_{0-∞}−AUC₀₋ₜ)/AUC_{0-∞};
CL/F: Apparent total clearance (as dose/AUC_{0-∞}); and
V_{z}/F: Apparent volume of distribution during the terminal phase (as dose/[λ_{z}*AUC_{0-∞}]).

FIG. 1 displays the plots of mean plasma bardoxolone methyl concentrations by treatment on linear and semi-logarithmic scales for the PK Concentration Population. Table 26 summarizes the plasma bardoxolone methyl PK parameters for the PK Parameter Population.

TABLE 26
Summary of Plasma Bardoxolone Methyl Pharmacokinetic
Parameters - Pharmacokinetic Parameter Population

| | | |
|---|---|---|
| PK Parameter (Unit) | Period 1 | Period 2 |

Cₘₐₓ (ng/mL)

| n | 16 | | 15 | |
|---|---|---|---|---|
| Mean (SD) | 2.58 | (0.889) | 17.3 | (5.20) |
| GM (Geom. CV %) | 2.43 | (38.0) | 16.6 | (29.3) |

tₘₐₓ (h)

| n | 16 | | 15 | |
|---|---|---|---|---|
| Mean (SD) | 2.9 | (1.1) | 3.1 | (0.8) |
| Median (min, max) | 2.5 | (2.0, 5.0) | 3.0 | (2.0, 5.0) |

λ_{z} (1/h)

| n | 16 | | 15 | |
|---|---|---|---|---|
| Mean (SD) | 0.0392 | (0.0143) | 0.00762 | (0.00218) |
| GM (Geom. CV %) | 0.0370 | (35.4) | 0.00740 | (24.1) |

t½ (h)

| n | 16 | | 15 | |
|---|---|---|---|---|
| Mean (SD) | 19.8 | (6.6) | 95.9 | (19.5) |
| Median (min, max) | 20.3 | (10.1, 36.4) | 97.7 | (48.1, 121.0) |

AUC₀₋ₜ (h*ng/mL)

| n | 16 | | 15 | |
|---|---|---|---|---|
| Mean (SD) | 29.3 | (13.8) | 226 | (55.1) |
| GM (Geom. CV %) | 26.1 | (53.8) | 220 | (24.7) |

AUC_{0-∞} (h*ng/mL)

| n | 16 | | 15 | |
|---|---|---|---|---|
| Mean (SD) | 31.0 | (14.3) | 248 | (62.7) |
| GM (Geom. CV %) | 27.8 | (52.1) | 241 | (25.4) |

AUC extra % (%)

| n | 16 | | 15 | |
|---|---|---|---|---|
| Mean (SD) | 6.10 | (2.04) | 8.47 | (3.30) |
| GM (Geom. CV %) | 5.81 | (33.0) | 7.64 | (56.9) |

CL/F (L/h)

| n | 16 | | 15 | |
|---|---|---|---|---|
| Mean (SD) | 402 | (202) | 42.8 | (10.6) |
| GM (Geom. CV %) | 359 | (52.1) | 41.6 | (25.4) |

V_{z}/F (L)

| n | 16 | | 15 | |
|---|---|---|---|---|
| Mean (SD) | 10200 | (3170) | 5900 | (1930) |
| GM (Geom. CV %) | 9700 | (34.5) | 5620 | (33.5) |

Note 1:
Period 1 was a single dose of bardoxolone methyl. Period 2 was a single dose of bardoxolone methyl and daily doses of itraconazole.
Note 2:
Geom. CV % = 100*(exp(SD2) − 1)0.5, where SD is the standard deviation of the logarithm-transformed data.
Note 3:
The Cₘₐₓ, AUC₀₋ₜ, AUC_{0-∞}, AUC extra %, λ_{z}, CL/F, and V_{z}/F values presented in-text were rounded to 3 significant figures, where appropriate. Unrounded source values are presented in the post-text table.
λ_{z} = first-order elimination rate constant calculated from the slope of logarithmic-linear regression of the terminal phase; AUC_{0-∞} = area under the plasma concentration versus time curve from time 0 extrapolated to infinity time, calculated as AUC₀₋ₜ + Cₗₐₛₜ/λ_{z}; AUC₀₋ₜ = area under the plasma concentration versus time curve from time 0 to the last quantifiable concentration; AUC extra % = percent extrapolated area under the plasma concentration versus time curve calculated as 100*(AUC_{0-∞} − AUC₀₋ₜ)/AUC_{0-∞}; CL/F = apparent total clearance (as dose/AUC_{0-∞}); Cₗₐₛₜ = the last quantifiable concentration; Cₘₐₓ = maximum plasma concentration observed directly from data; Geom. CV % = geometric coefficient of variation; GM = geometric mean; max = maximum; min = minimum; PK = pharmacokinetic(s); SD = standard deviation; t½ = terminal elimination half-life, calculated as ln(2)/λ_{z}; tₘₐₓ = time to reach the maximum concentration directly from data; V_{z}/F = apparent volume of distribution during the terminal phase (as dose/[λ_{z}*AUC_{0-∞}]).

The effect of itraconazole on plasma bardoxolone methyl PK parameters was assessed by a paired t-test. A subject must have had a calculable PK parameter in both periods in order to be included in the analysis for that parameter. Two-sided 90% CIs for the GM ratios of Cₘₐₓ, AUC₀₋ₜ, and AUC_{0-∞} (test versus reference) were calculated using a paired t-test for the natural logarithm-transformed PK parameters. The 90% CIs were calculated after back transformation to the original scale. Based on a guidance from the FDA, if the 90% CIs of the GMs of the systemic exposure PK parameters (Cₘₐₓ, AUC₀₋ₜ, and AUC_{0-∞}) fell entirely within the equivalence range of 80% to 125%, then there was no clinically significant drug-drug interaction.

Table 27 summarizes the analysis of the effect of itraconazole on plasma bardoxolone methyl PK parameters for the PK Parameter Population. The GM % ratios ([bardoxolone methyl co-administered with itraconazole]/[bardoxolone methyl alone]) for bardoxolone methyl Cₘₐₓ, AUC₀₋ₜ, and AUC_{0-∞} (with 90% CIs) were 689.77% (552.87, 860.57), 851.76% (682.64, 1062.78), and 872.54% (708.20, 1075.01), respectively. The 90% CIs for all GM ratios were outside of the pre-determined equivalence range of 80% to 125% range.

TABLE 27
Analysis of the Effect of Itraconazole on Plasma Bardoxolone Methyl
Pharmacokinetic Parameters - Pharmacokinetic Parameter Population

| | Bardoxolone Methyl | | Bardoxolone Methyl + | | | |
|---|---|---|---|---|---|---|
| PK | Alone (A) | | Itraconazole (B) | | Ratio | 90% CI |
| Parameter | | Geom. | | Geom. | B/A | for Ratio^{a} |
| (Unit) | n | Mean^{b} | n | Mean^{b} | (%) | (%) |
| Cₘₐₓ | 15 | 2.41 | 15 | 16.6 | 689.77 | (552.87, |
| (ng/mL) | | | | | | 860.57) |
| AUC₀₋ₜ | 15 | 25.8 | 15 | 220 | 851.76 | (682.64, |
| (h*ng/mL) | | | | | | 1062.78) |
| AUC_{0-∞} | 15 | 27.6 | 15 | 241 | 872.54 | (708.20, |
| (h*ng/mL) | | | | | | 1075.01) |

Note 1:
A subject must have had a calculable PK parameter in both periods in order to be included in the analysis for that parameter.
Note 2:
The Cₘₐₓ, AUC₀₋ₜ, and AUC_{0-∞} geom. mean values presented in-text were rounded to 3 significant figures, where appropriate. Unrounded source values are presented in the post-text table.
^{a}Two-sided 90% CIs for geom. mean ratios of PK parameters (test versus reference) were calculated using a paired t-test for the natural logarithm-transformed PK parameters. The 90% CIs were presented after back transformation to the original scale.
^{b}Geometric means were presented after back transformation to the original scale.
λ_{z} = first-order elimination rate constant calculated from the slope of logarithmic-linear regression of the terminal phase; AUC_{0-∞} = area under the plasma concentration versus time curve from time 0 extrapolated to infinity time, calculated as AUC₀₋ₜ + Cₗₐₛₜ/λ_{z}; AUC₀₋ₜ = area under the plasma concentration versus time curve from time 0 to the last quantifiable concentration; CI = confidence interval; Cₗₐₛₜ = the last quantifiable concentration; Cₘₐₓ = maximum plasma concentration observed directly from data; geom. = geometric; PK = pharmacokinetic(s).

Following oral administration, bardoxolone methyl was rapidly absorbed either when administered alone or in combination with a strong CYP3A4 inhibitor (itraconazole) with a median time to reach Cₘₐₓ directly from data of 2.5 hours to 3 hours. The Cₘₐₓ and total exposure (AUC₀₋ₜ and AUC_{0-∞}) of bardoxolone methyl increased following concomitant administration of a single oral dose of bardoxolone methyl and itraconazole compared to a single oral dose of bardoxolone methyl alone. The GM % ratios ([bardoxolone methyl co-administered with itraconazole]/[bardoxolone methyl alone]) were 689.77%, 851.76%, and 872.54% for Cₘₐₓ, AUC₀₋ₜ, and AUC_{0-∞}, respectively. The corresponding 90% CIs of Cₘₐₓ, AUC₀₋ₜ, and AUC_{0-∞} ([552.87, 860.57], [682.64, 1062.78], and [708.20, 1075.01], respectively) were outside of the pre-determined equivalence range of 80% to 125%, indicating a PK drug-drug interaction. An increase of >5-fold in the exposure of bardoxolone methyl in the presence of itraconazole indicated that bardoxolone methyl is a sensitive substrate of CYP3A4.

The mean t_{1/2} of bardoxolone methyl was prolonged from 19.8 hours (bardoxolone methyl alone) to 95.9 hours (bardoxolone methyl+itraconazole) when bardoxolone methyl was administered concomitantly with itraconazole. Additionally, the clearance (GM CL/F) of bardoxolone methyl was reduced from 359 L/h (bardoxolone methyl alone) to 41.6 L/h (bardoxolone methyl+itraconazole) when bardoxolone methyl was administered concomitantly with itraconazole. These results further supported the PK drug-drug interaction between bardoxolone methyl and itraconazole.

The Cmax and total exposure (AUC₀₋ₜ and AUC_{0-∞}) of bardoxolone methyl increased following concomitant administration of bardoxolone methyl and itraconazole compared with bardoxolone methyl alone. The 90% CIs for the GM ratios of Cₘₐₓ, AUC₀₋ₜ, and AUC_{0-∞} for bardoxolone methyl were outside of the pre-determined equivalence range of 80% to 125%, indicating a PK drug-drug interaction. An increase of >5-fold in the exposure of bardoxolone methyl in the presence of itraconazole indicated that bardoxolone methyl is a sensitive substrate of CYP3A4.

Safety evaluation and results: The Safety Population included all subjects who received at least 1 dose of bardoxolone methyl. Safety analyses were performed based on the Safety Population. Safety assessments included demographic/medical history, adverse event monitoring, vital signs, physical examinations, 12-lead ECGs, clinical laboratory tests, and viral serology. Safety data were summarized by treatment segment.

A total of 15 subjects completed the study per protocol and received a single dose of bardoxolone methyl alone (Period 1), 12 daily doses of itraconazole alone (Period 2), and a dose of itraconazole followed 1 hour later by a dose of bardoxolone methyl (Period 2). One subject (Subject 1797-002) withdrew early from the study (due to a family emergency) after receiving a single oral dose of bardoxolone methyl but prior to completing Period 1.

Concomitant administration of bardoxolone methyl with a strong CYP3A4 inhibitor (itraconazole) was generally safe and well tolerated in this study. In total, 3 (18.8%) subjects had a TEAE following administration of bardoxolone methyl alone (bardoxolone methyl) (Period 1), 1 (6.7%) subject had a TEAE following administration of itraconazole alone (itraconazole) (Period 2), and 1 (6.7%) subject had a TEAE following concomitant administration of bardoxolone methyl and itraconazole (bardoxolone methyl+itraconazole) (Period 2).

No subjects died, had an SAE, or had an adverse event that led to discontinuation of study drug during the study. No specific TEAEs occurred in more than 1 subject. All TEAEs were Grade 1 in severity. The majority of TEAEs were assessed as unrelated to study drug by the investigator. In total, 1 (6.3%) subject had study drug-related TEAEs of diarrhea, nausea, and headache following administration of bardoxolone methyl alone (bardoxolone methyl) (Period 1). No other subjects had any study drug-related TEAEs.

There were no clinically relevant mean or individual changes in laboratory parameters during the study. Additionally, there were no clinically relevant shifts in vital signs, weight, or 12-lead ECG findings during the study. There were also no clinically relevant physical examination findings during the study.

### Example 2—Effect of CYP3A4 Inhibitors on Omaveloxolone Pharmacokinetics

Omaveloxolone (RTA 408) and related triterpenoid analogs are among the most potent known activators of nuclear factor erythroid-derived 2-related factor 2 (Nrf2) and are also inhibitors of nuclear factor kappa-light-chain enhancer of activated B-cells and thus induce an anti-inflammatory and anti-oxidative phenotype. Because Nrf2 signaling promotes anti-oxidative mechanisms (Muthusamy et al., 2012) and Nrf2 activation can increase mitochondrial respiration (Holmostrom et al., 2013; Ludtmann et al., 2014). Reata hypothesizes that the anti-oxidative and anti-inflammatory effects, along with improved mitochondrial function, contribute to the efficacy of omaveloxolone in a number of diseases such as Friedreich's ataxia (FA).

Patients with FA have impaired adenosine triphosphate (ATP) production and phosphocreatine (PCr) regeneration as a result of reactive oxygen species (ROS)-induced damage to the mitochondria (Nachbauer et al., 2013). This impaired ATP and PCr production likely contributes to the progressive muscle weakness, exercise intolerance, and fatigue observed in these patients, as well as other disease manifestations, such as visual impairment.

Recent studies have shown that activity of the Kelch-like ECH-associated protein-1 (Keap1) Nrf2 pathway, which responds to oxidative stress, is decreased in patients with FA. Specifically, cultured cells from patients with FA exhibit hypersensitivity to oxidative insults because of impairment in the Nrf2 signaling pathway and corresponding decreases in expression of Nrf2-mediated endogenous antioxidants, such as glutathione, NAD(P)H:quinone oxidoreductase 1 (NQO1), and superoxide dismutase (Paupe et al., 2009).

Several lines of evidence suggest that Nrf2 activation can increase mitochondrial respiration and biogenesis, as well as the induction of numerous anti-oxidative genes to counter the pathological ROS generated by dysfunctional iron handling (Paupe et al., 2009; Shan et al., 2013; D'Oria et al., 2013). Consistent with these findings, non-clinical data have demonstrated that omaveloxolone affords significant protection from hydrogen peroxide-induced oxidative stress in cerebral granule neurons (Abeti et al., 2018).

Omaveloxolone is currently being evaluated in a clinical trial for the treatment of FA. Omaveloxolone has also been evaluated in clinical trials for cancer, mitochondrial myopathies, prevention of radiation-induced dermatitis with topical lotion administration, and for the prevention and treatment of ocular inflammation and corneal endothelial cell loss in patients following ocular surgery with topical ocular administration.

This study was designed to determine the effect of CYP3A4 inhibition by strong inhibitor of CYP3A4 (itraconazole) on the PK of omaveloxolone. In vitro data from experiments with human liver microsomes and recombinantly-expressed CYPs have shown that omaveloxolone is metabolized by CYP3A4. In human liver microsomes, disappearance of omaveloxolone is inhibited by the CYP3A4/5 inhibitor ketoconazole (1 μM) up to 99%, with Kₘ and Vmax values 1.12 μM and 717 pmol/mg/min, respectively. Further, the kinetic constants for omaveloxolone in recombinant CYP3A4 (rCYP3A4) are summarized in the table below:

Enzyme Kinetic Constants in rCYP3A4 for Omaveloxolone

| | Kₘ | Vₘₐₓ | CLᵢₙₜ |
|---|---|---|---|
| Compound | (μM) | (pmol/min/pmol CYP) | (μL/min/pmol CYP) |
| Omaveloxolone | 1.17 | 19.1 | 16.3 |

Thus, a reduction in the functional capacity of CYP3A4 enzymes might impact the systemic exposure to bardoxolone methyl. Itraconazole is a potent inhibitor of CYP3A4, and is, therefore, a good probe to study the potential impact of CYP3A4 inhibition (Liu, 2016; Ke, 2014). The primary objective of this study was to determine the impact of multiple oral doses of itraconazole on the single oral dose PK of omaveloxolone in healthy subjects. The secondary objective of this study was to determine the safety of omaveloxolone alone and in combination with itraconazole in healthy subjects.

This was a Phase 1, open-label, fixed-sequence, drug-drug interaction study in healthy male and female subjects. Potential subjects were screened to assess their eligibility to enter the study within 28 days prior to the first dose administration. Subjects were admitted into the CRU on Day −1 and were confined to the CRU until discharge on Day 23. A post-study assessment was performed prior to discharge. In all parts subjects received a safety Follow-up phone call 5 to 7 days after discharge from the CRU. The total duration of study participation for each subject (from Screening through the Follow-up phone call) was approximately 8 weeks.

An overview of the study design is shown in FIG. 2. A total of 15 subjects were enrolled to ensure that 12 subjects completed the study. All subjects were enrolled in a single group and received the following treatments in 1 treatment period: Days 1 and 13: oral doses of 150 mg omaveloxolone QD; Days 10 to 18: oral doses of 200 mg itraconazole QD.

Healthy males or females, of any race, aged between 18 and 55 years, inclusive, and with a body mass index (BMI) between 18.0 and 32.0 kg/m², inclusive, were selected according to the inclusion and exclusion criteria listed. Each dose of omaveloxolone and itraconazole was administered orally, with approximately 240 mL of room temperature water.

To match the intended clinical dose, oral doses of 150 mg omaveloxolone were chosen for the current study. This dose allowed a 2-fold safety margin compared to the highest evaluated dose of 300 mg (QD for up to 12 weeks) in previous studies, in case of substantial inhibition of omaveloxolone metabolism by the probe drugs. The selected doses of itraconazole was based on typical therapeutic doses for this drugs, and was considered to be high enough to provide sufficient plasma concentrations to achieve the objectives of the study.

Subjects refrained from the use of any prescription or nonprescription medications/products during the study until after the Follow-up phone call, unless the investigator (or designee) and/or Sponsor had given their prior consent.

Paracetamol/acetaminophen (2 g/day for up to 3 consecutive days) was an acceptable concomitant medication. The administration of any other concomitant medications during the study was prohibited without prior approval of the investigator (or designee), unless its use was deemed necessary for treatment of an AE. Any medication taken by a subject during the course of the study and the reason for its use were documented in the source data.

Ginger ale and prune juice were to be provided to the subject as necessary, without the need to be recorded as a concomitant medication.

While confined at the CRU, subjects received a standardized diet at scheduled times that did not conflict with other study-related activities. In addition, subjects were required to comply with the following restrictions: Foods and beverages containing poppy seeds, grapefruit, or Seville oranges were not allowed from 7 days prior to Check-in until discharge from the CRU. Caffeine-containing foods and beverages were not allowed from 48 hours before Check-in until discharge from the CRU. Consumption of alcohol was not permitted from 72 hours prior to Check-in until discharge from the CRU.

Subjects were not permitted to use tobacco- or nicotine-containing products within 3 months prior to Check-in until discharge from the CRU.

The planned assessments and their timing in relation to dosing are listed in Table 28.

TABLE 28
Schedule of assessments.

| Study | | Study Day | | | | | | | | | | |
|---|---|---|---|---|---|---|---|---|---|---|---|---|
| Procedure | Screening | −1 | 1 | 2 | 3 | 4 | 5 | 6 | 7 | 8 | 9 | 10 |
| Informed consent | X | | | | | | | | | | | |
| Inclusion/exclusion | X | X | | | | | | | | | | |
| criteria | | | | | | | | | | | | |
| Demographic data | X | | | | | | | | | | | |
| Medical history | X | X^{b} | | | | | | | | | | |
| Drug screen | X | X^{c} | | | | | | | | | | |
| B-type natriuretic | X | | | | | | | | | | | |
| peptide test | | | | | | | | | | | | |
| Serology | X | | | | | | | | | | | |
| FSH^{d} | X | | | | | | | | | | | |
| Pregnancy test^{d} | X | X | | | | | | | | | | |
| Height and weight* | X | X | | | | | | | | | | |
| Study residency: | | | | | | | | | | | | |
| Check-in | | X | | | | | | | | | | |
| Check-out | | | | | | | | | | | | |
| Non-residential | X | | | | | | | | | | | |
| visit | | | | | | | | | | | | |
| Study drug | | | | | | | | | | | | |
| administration: | | | | | | | | | | | | |
| Omaveloxolone | | | X | | | | | | | | | |
| Iraconazole^{f} | | | | | | | | | | | | X |
| Pharmacokinetic | | | | | | | | | | | | |
| blood samples: | | | | | | | | | | | | |
| Omaveloxolone^{h} | | | X | X | X | X | X | X | X | X | X | X |
| Itraconazoleⁱ | | | | | | | | | | | | |
| Safety and | | | | | | | | | | | | |
| tolerability: | | | | | | | | | | | | |
| Adverse event | X | X | X | | | | | | | | | |
| recording | | | | | | | | | | | | |
| Prior/concomitant | X | X | X | | | | | | | | | |
| medication | | | | | | | | | | | | |
| monitoring | | | | | | | | | | | | |
| Clinical laboratory | X | X | | 24^{l} | 48^{l} | | | | | | X | |
| evaluation (hours)^{j} | | | | | | | | | | | | |
| Vital signs | X | | P, 4, | 24^{l} | 48^{l} | | | | | | | P |
| (hours)^{k} | | | 12^{l} | | | | | | | | | |
| Triplicate | X | | P, 4, | 24^{l} | 48^{l} | | | | | | | P |
| 12-lead | | | 12^{l} | | | | | | | | | |
| ECG (hours) | | | | | | | | | | | | |
| Physical | X | X | | | | | | | | | | |
| examination | | | | | | | | | | | | |

| Study | Study Day | | | | | | | | | | Follow- |
|---|---|---|---|---|---|---|---|---|---|---|---|
| Procedure | 11 | 12 | 13 | 14 | 15 | 16 | 17 | 18 | 19-22 | 23/ED | Up^{a} |
| Informed consent | | | | | | | | | | | |
| Inclusion/exclusion | | | | | | | | | | | |
| criteria | | | | | | | | | | | |
| Demographic data | | | | | | | | | | | |
| Medical history | | | | | | | | | | | |
| Drug screen | | | | | | | | | | | |
| B-type natriuretic | | | | | | | | | | | |
| peptide test | | | | | | | | | | | |
| Serology | | | | | | | | | | | |
| FSH^{d} | | | | | | | | | | | |
| Pregnancy test^{d} | | | | | | | | | | X | |
| Height and weight* | | | | | | | | | | X | |
| Study residency: | | | | | | | | | | | |
| Check-in | | | | | | | | | | | |
| Check-out | | | | | | | | | | X | |
| Non-residential | | | | | | | | | | | |
| visit | | | | | | | | | | | |
| Study drug | | | | | | | | | | | |
| administration: | | | | | | | | | | | |
| Omaveloxolone | | | X | | | | | | | | |
| Iraconazole^{f} | X | X | X | X | X | X | X | X | | | |
| Pharmacokinetic | | | | | | | | | | | |
| blood samples: | | | | | | | | | | | |
| Omaveloxolone^{h} | X | | X | X | X | X | X | X | X | X^{g} | |
| Itraconazoleⁱ | X | X | X | X | X | | X | | | | |
| Safety and | | | | | | | | | | | |
| tolerability: | | | | | | | | | | | |
| Adverse event | X | | | | | | | | | | X |
| recording | | | | | | | | | | | |
| Prior/concomitant | X | | | | | | | | | | X |
| medication | | | | | | | | | | | |
| monitoring | | | | | | | | | | | |
| Clinical laboratory | | | P | 24^{l} | 48^{l} | | | X | | X | |
| evaluation (hours)^{j} | | | | | | | | | | | |
| Vital signs | | | P, 4, | 24^{l} | 48^{l} | | | | | X | |
| (hours)^{k} | | | 12^{l} | | | | | | | | |
| Triplicate | | | P, 4, | 24^{l} | 48^{l} | | | | | X | |
| 12-lead | | | 12^{l} | | | | | | | | |
| ECG (hours) | | | | | | | | | | | |
| Physical | | | | | | | | | | X^{m} | |
| examination | | | | | | | | | | | |

Abbreviations: ECG = electrocardiogram; ED = early discontinuation; FSH = follicle-stimulating hormone; P = predose; PK = Pharmacokinetc; QD = once daily.
^{a}Follow-up phone call was 5 to 7 days after discharge from the clinical research unit.
^{b}Interim medical history.
^{c}Drug screen did not include alcohol testing at Screening and did include alcohol testing at Check-in.
^{d}FSH testing for postmenopausal females only. Serum pregnancy testing for all females.
^{e}Height measured at Screening only.
^{f}Dosing QD each day.
^{g}Day 23 only.
^{h}Omaveloxolone PK timepoints for Days 1 and 13: pre-omaveloxolone dose, and 0.5, 1, 1.5, 2, 3, 4, 6, 8, 12, 16, 24, 36, 48, 72, 96, 120, 144, 168, 192, 216, and 240 hours post-omaveloxolone dose.
ⁱItraconazole PK blood samples were taken pre-itraconazole dose on identified days.
^{j}Clinical laboratory evaluation samples were taken following a fast of at least 8 hours.
^{k}Blood pressure, pulse rate, respiratory rate, and oral body temperature.
^{l}Timepoints relative to omaveloxolone dosing.
^{m}Symptom-directed physical examination prior to discharge.

A total of 15 subjects were enrolled and dosed in the study. Fourteen subjects had a total exposure of 300 mg omaveloxolone administered as 2 single 150-mg doses, and a total exposure of 1800 mg itraconazole (9×200-mg doses) administered QD over 9 days. One subject was withdrawn from the study, due to an AE of urinary retention, before receiving all planned doses and had a total exposure of 150 mg omaveloxolone and 400 mg itraconazole (2×200-mg doses) administered QD for 2 days. One subject withdrew their consent.

A detailed summary of screening demographic data for subjects receiving each of the study treatments is presented in Table 29. Subjects were males and females aged 22 to 55 years, inclusive, and had a BMI between 18.7 and 31.9 kg/m2, inclusive.

TABLE 29
Summary of Screening Demographic Data

| | | | |
|---|---|---|---|
| Age (years) | Mean (SD) | 41 | (11.4) |
| Sex | Male | 10 | (66.7%) |
| | Female | 5 | (33.3%) |
| Race | Black or African American | 7 | (46.7%) |
| | White | 8 | (53.3%) |
| Weight (kg) | Mean (SD) | 80.1 | (11.26) |
| BMI (kg/m²) | Mean (SD) | 28.0 | (3.33) |

Abbreviations: BMI = body mass index; SD = standard deviation.

Pharmacokinetics evaluation and results: The PK outcome endpoints of omaveloxolone derived from the plasma concentration-time profile following oral administration alone and in combination with itraconazole were:
AUC_{0-∞}: AUC from time 0 extrapolated to infinity time, calculated as AUC₀₋ₜ+Cₗₐₛₜ/λ_{z};
Cₘₐₓ: Maximum plasma concentration observed directly from data;
tₘₐₓ: Time to reach Cₘₐₓ directly from data;
t_{1/2}: Terminal elimination half-life, calculated as ln(2)/λ_{z};
AUC₀₋ₜ: AUC from time 0 to the last quantifiable concentration (Cₗₐₛₜ);
CL/F: Apparent total clearance (as dose/AUC_{0-∞}); and
V_{z}/F: Apparent volume of distribution during the terminal phase (as dose/[λ_{z}*AUC_{0-∞}]).

Arithmetic mean plasma concentration-time profiles of omaveloxolone following administration alone and co-administration with itraconazole are presented in FIG. 3.

The summary and statistical analysis of PK parameters for omaveloxolone alone and in combination with itraconazole are presented in Table 30 and Table 31, respectively. High geometric mean trough concentrations of itraconazole were detected from Day 12 to Day 17, indicating marked exposure to itraconazole was maintained prior to and following co-administration with omaveloxolone.

TABLE 30
Summary of the Pharmacokinetic Parameters of Omaveloxolone

| | Omaveloxolone | | | |
|---|---|---|---|---|
| | | | 200 mg itraconazole | |
| | 150 mg | | QD + 150 mg | |
| | omaveloxone QD | | omaveloxone QD | |
| Parameter | (Reference) | | (Test) | |
| AUC₀₋ₜ (h*ng/mL) | 1730 | (64.8) | 6340 | (39.9) |
| AUC_{0-∞} (h*ng/mL) | 1730 | (50.7) | 7280 | (48.7) |
| Cₘₐₓ (ng/mL) | 43.0 | (41.8) | 116 | (69.5) |
| tₘₐₓ^{a} (h) | 12.00 | (2.00-24.00) | 6.00 | (2.00-36.00) |
| t_{1/2} (h) | 89.8 | (30.5) | 78.1 | (49.9) |
| CL/F (L/h) | 86.7 | (50.7) | 20.6 | (48.7) |
| V_{z}/F (L) | 11200 | (55.1) | 2320 | (31.4) |

Geometric mean (CV %) data are presented.
Abbreviations: AUC₀₋ₜ = area under the plasma concentration-time curve from time zero to the time of last quantifiable concentration; AUC_{0-∞} = area under the plasma concentration-time curve from time zero to infinity; CL/F = apparent total plasma clearance; Cₘₐₓ = maximum observed plasma concentration; CV = coefficient of variation; N = number of subjects studied; QD = once daily; t_{1/2} = apparent plasma terminal elimination half-life; Tₘₐₓ = time of maximum observed plasma concentration; V_{z}/F = apparent total volume of distribution during the terminal elimination phase.
^{a}Median (min-max).

TABLE 31
Statistical Analysis of the Pharmacokinetic Parameters
of Omaveloxolone (200 mg itraconazole QD + 150 mg
omaveloxone QD (Test) vs 150 mg omaveloxolone QD (Reference))

| | | | | | Ratio of GLSMs | Within- |
|---|---|---|---|---|---|---|
| | Test | | Reference | | (90% CI) | subject |
| Parameter | n | GLSM | n | GLSM | (Test:Reference) | CV % |
| AUC_{0-∞} | 13 | 7130 | 14 | 1730 | 4.12 | 24.4 |
| (h*ng/mL) | | | | | (3.48, 4.87) | |
| Cₘₐₓ | 13 | 119 | 15 | 43.0 | 2.77 | 36.8 |
| (ng/mL) | | | | | (2.17, 3.54) | |
| tₘₐₓ | 13 | 6.00 | 15 | 8.00 | 5.00 | |
| (h)# | | | | | (−4.00, 12.0) | |

Abbreviations: AUC_{0-∞} = area under the concentration time curve from time zero extrapolated to infinity; CI = confidence interval; Cₘₐₓ = maximum observed plasma concentration; GLSM = geometric least squares mean; n = number of observations; Tₘₐₓ = time of maximum observed plasma concentration; QD = once daily Model: ln(parameter) = treatment + subject + random error, with subject fitted as a random effect The ratio and corresponding confidence limits are back-transformed from the difference and confidence limits calculated on the loge scale.
#The n, median, and Hodges-Lehmann estimate of median difference (90% CI) from the Wilcoxon signed rank test are presented.

Following a single dose of 150 mg omaveloxolone administered alone, the plasma concentration-time profile indicated a slow absorption, which was also observed following a single dose of 150 mg omaveloxolone co-administered with 200 mg itraconazole. Individual Tmax values were highly variable yet median Tmax (min-max) was similar following administration of omaveloxolone alone compared to co-administration of omaveloxolone with itraconazole (12.00 [2.00-24.00] and 6.00 [2.00-36.00] hours, respectively). After Cmax, a smaller secondary peak was observed at approximately 24 hours postdose, following which plasma concentrations of omaveloxolone declined slowly in a generally multiphasic manner. The geometric mean t1/2 was similar when omaveloxolone was administered alone (89.8 hours) and when co-administered with itraconazole (78.1 hours). Between-subject variability was high for omaveloxolone administered alone and when co-administered with itraconazole, based on AUC0-∞ and Cmax (geometric CV % ranging from 41.8% to 69.5%) (Table 30).

Following co-administration of 150 mg omaveloxolone with multiple doses of 200 mg itraconazole, there was an increase in systemic omaveloxolone exposure compared to administration of omaveloxolone alone, based on AUC0-∞ and Cmax; the ratios (90% CI) of geometric LS means were 4.12 (3.48, 4.87) and 2.77 (2.17, 3.54), respectively (Table 31).

Within-subject variability of omaveloxolone AUC0-∞ and Cmax was low to moderate, with geometric CV % values of 24.4% and 36.8%, respectively (FIG. 3, Table 31).

An increase in systemic exposure of omaveloxolone (approximately 4- and 3-fold in AUC0-∞ and Cmax, respectively) was observed following co-administration with itraconazole compared to omaveloxolone administered alone. As itraconazole is a strong inhibitor of CYP3A4, the results from the current study are consistent with in vitro findings, where CYP3A4 was implicated as the major contributing enzyme to omaveloxolone metabolism.

Safety evaluation and results: The Safety Population included all subjects who received at least 1 dose of omaveloxolone. Safety analyses were performed based on the Safety Population. Safety assessments included demographic/medical history, adverse event monitoring, vital signs, physical examinations, 12-lead ECGs, clinical laboratory tests, and viral serology. Safety data were summarized by treatment segment.

A total of 2 subjects (13.3%) reported TEAEs following administration of omaveloxolone alone, whereas no subjects reported TEAEs following administration of omaveloxolone in combination with itraconazole. One subject experienced a TEAE of urinary retention on Day 11 following administration of a single oral dose of 150 mg omaveloxolone and 2 single oral doses of 200 mg itraconazole. Study treatment was permanently discontinued following onset of the AE and early termination procedures were performed on Day 23. The TEAE was considered moderate in severity and unrelated to omaveloxolone or itraconazole, by the investigator. There were no deaths or SAEs during the study. There were no safety concerns when omaveloxolone was administered alone or in combination with itraconazole. There were no clinically significant findings in clinical laboratory evaluations, vital signs, and ECG parameters.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,480,792
U.S. Pat. No. 5,525,524
U.S. Pat. No. 5,631,170
U.S. Pat. No. 5,679,526
U.S. Pat. No. 5,824,799
U.S. Pat. No. 5,851,776
U.S. Pat. No. 5,885,527
U.S. Pat. No. 5,922,615
U.S. Pat. No. 5,939,272
U.S. Pat. No. 5,947,124
U.S. Pat. No. 5,955,377
U.S. Pat. No. 5,985,579
U.S. Pat. No. 6,019,944
U.S. Pat. No. 6,113,855
U.S. Pat. No. 6,143,576
U.S. Pat. No. 8,129,429
U.S. Pat. Pub. 2003/0232786
U.S. Pat. Pub. 2008/0261985
U.S. Pat. Pub. 2009/0048204
U.S. Pat. Pub. 2009/0060873
U.S. Pat. Pub. 2009/0326063
U.S. Pat. Pub. 2010/0041904
U.S. Pat. Pub. 2010/0048887
U.S. Pat. Pub. 2010/0048892
U.S. Pat. Pub. 2010/0048911
U.S. Pat. Pub. 2010/0056777
U.S. Pat. Pub. 2011/0201130
PCT Pub. WO 2009/023232
PCT Pub. WO 2010/093944
PCT Pub. WO 2013/163344
Abraham and Kappas, Free Radical Biol. Med., 39:1-25, 2005.
Abeti R, Baccaro A, Esteras N, Giunti P. Novel Nrf2-Inducer Prevents Mitochondrial Defects and Oxidative Stress in Friedreich's Ataxia Models. Front Cell Neurosci. 2018; 12:188.
Aghajan et al., J Gastroenterol Hepatol., Suppl 2:10-14, 2012.
Ahmad et. al., Cancer Res., 68:2920-2926, 2008.
Ahmad et. al., J. Biol. Chem., 281:35764-9, 2006.
Angulo et al., Eur. J. Immunol., 30:1263-1271, 2000.
Araujo et. al., J. Immunol., 171(3):1572-1580, 2003.
Arend and Dayer, Arthritis Rheum., 38:151-160, 1995.
Bach, Hum. Immunol., 67(6):430-432, 2006.
Bagasra et al., Proc. Natl. Acad. Sci. USA, 92:12041-12045, 1995.
Botoman et al., Am. Fam. Physician, 57(1):57-68, 1998.
Brandt et al., Arthritis Rheum., 43:1346-1352, 2000.
Brewerton et al., Lancet., 1:904-907, 1973a.
Brewerton et al., Lancet., 1:956-957, 1973b.
Bronte et al., Trends Immunol., 24:302-306, 2003.
Brown and DuBois J. Clin. Oncol., 23:2840-2855, 2005.
Brynskov et al., N. Engl. J. Med., 321(13):845-850, 1989.
Cai et al., Nat. Med., 11(2):183-190, 2005.
Calin and Taurog, In: The Spondylarthritides, Calin et al. (Eds.), Oxford, UK. Oxford University Press, 179, 1998.
Chauhan and Chauhan, Pathophysiology, 13(3):171-181. 2006.
Crowell et al., Mol. Cancer Ther., 2:815-823, 2003.
Dhaun et al., American Journal of Physiology—Renal Physiology, 296:F1477-F1483, 2009.
Dickerson et. al., Prog Neuropsychopharmacol Biol. Psychiatry, Mar. 6, 2007.
Dinarello, Int. Rev. Immunol., 16:457-499, 1998.
Dinkova-Kostova et al., Proc. Natl. Acad. Sci. USA, 102(12):4584-4589, 2005.
Dionne et al., Clin. Exp. Imunol., 112(3):435-442, 1998.
D'Oria et al., Int. J. Mol. Sci., 14(4):7853-65, 2013.
Dudhgaonkar et. al., Eur. J. Pain, 10(7):573-9, 2006.
Eastgate et al., Lancet, 2(8613):706-9, 1988.
Eikelenboom et al., Glia, 40(2):232-239, 2002.
Ettehadi et al., Clin. Exp. Immunol., 96(1):146-151, 1994.
Forstermann, Biol. Chem., 387:1521, 2006.
Funakoshi et al., Digestion, 59(1):73-78, 1998.
Gehrmann et al., Glia, 15(2):141-151, 1995.
Genain and Nauser, J. Mol. Med., 75:187-197, 1997.
Goodman et al., Kidney Int., 72:945-953, 2007.
Graeber et al., Glia, 40(2):252-259, 2002.
Greten et al., Cell, 118:285-296, 2004.
Grivennikov and Karin, Cytokine Growth Factor Rev., 21(1):11-19, 2010.
Guilherme et al., Nat. Rev. Mol. Cell Biol., 9(5):367-77, 2008.
Gwee et al., Gut., 44(3):400-406, 1999.
Hahn and Tsao, In: Dubois' Lupus Erythematosus, 4^{th} Ed, Wallace and Hahn (Eds.), Lea and Febiger, Philadelphia, 195-201, 1993.
Handbook of Pharmaceutical Salts: Properties, and Use, Stahl and Wermuth Eds.), Verlag Helvetica Chimica Acta, 2002.
Hanson et. al., BMC Medical Genetics, 6(7), 2005.
Hansson et al., Annu. Rev. Pathol. Mech. Dis., 1:297-329, 2006.
He and Karin, Cell Res., 21(1):159-168, 2011.
Holmstrom et al., Biol. Open., 2(8):761-70, 2013.
Honda et al., Bioorg. Med. Chem. Lett., 12:1027-1030, 2002.
Honda et al., Bioorg. Med. Chem. Lett., 19:2711-2714, 1998.
Honda et al., Bioorg. Med. Chem. Lett., 9:3429-3434, 1999.
Honda et al., J. Med. Chem., 43:1866-1877, 2000a.
Honda et al., J. Med. Chem., 43:4233-4246, 2000b.
Honda et al., Med. Chem. Lett., 7:1623-1628, 1997.
Hotamisligil, Nature, 444(7121):860-7, 2006.
Huang et al., Cancer Res., 54:701-708, 1994.
Ishikawa et. al., Circulation, 104(15):1831-1836, 2001.
Ishizawa and Dickson, J. Neuropathol. Exp. Neurol., 60(6):647-657, 2001.
Jarvis, Curr. Opin. Rheumatol., 10(5):459-467, 1998.
Jarvis, Pediatr. Ann., 31(7):437-446, 2002.
Jonsson et al., Oral Dis., 8(3):130-140, 2002.
Jonsson et al., Trends Immunol., 22(12):653-654, 2001.
Kahle et al., Ann. Rheum. Dis., 51:731-734, 1992.
Kaltschmidt et al., Proc. Natl. Acad. Sci. USA, 94:2642-2647, 1997.
Karin, Nature, 441(7092):431-436, 2006.
Kawakami et. al., Brain Dev., 28(4):243-246, 2006.
Ke et al., Clin. Pharmacol. Ther., 95:473-476, 2014.
Kendall-Tackett, Trauma Violence Abuse, 8(2):117-126, 2007.
Kortylewski et al., Nat. Med., 11:1314-1321, 2005.
Kotzin and O'Dell, In: Samler's Immunologic Diseases, 5^{th} Ed., Frank et al. (Eds.), Little Brown & Co., Boston, 667-697, 1995.
Kotzin, Cell, 85:303-306, 1996.
Kruger et. al., J. Pharmacol. Exp. Ther., 319(3):1144-1152, 2006.
Kuboyama, Kurume Med. J., 45(1):33-37, 1998.
Lee et. al., Glia., 55(7):712-22, 2007.
Lencz et. al., Mol. Psychiatry, 12(6):572-80, 2007.
Liby et al., Cancer Res., 65:4789-4798, 2005.
Lipsky, In: Harrison's principles of internal medicine, Fauci et al.(Eds.), 14^{th} Ed., NY, McGraw-Hill, 1880-1888, 1998.
Liu et. al., FASEB J., 20(2):207-216, 2006.
Liu et al., J. Clin. Pharmacol., 56:143-151, 2016.
Ludtmann et al., Biochem J., 457(3):415-24, 2014.
Lugering et al., Ital. J. Gastroenterol. Hepatol., 30(3):338-344, 1998.
Mann et al., Avosentan for overt diabetic nephropathy. Journal of the American Society of Nephrology, 21:527-535, 2010.
March's Advanced Organic Chemistry: Reactions, Mechanisms, and Structure, 2007.
Mazur et al., Cell Microbiol., 9(7):1683-94, 2007.
Mazzoni et al., J Immunol., 168:689-695, 2002.
McAlindon et al., Gut, 42(2):214-219, 1998.
McGeer and McGeer, Brain Res. Brain Res. Rev., 21:195-218, 1995.
McGeer et al., Neurology, 19:331-338, 1996.
McGonagle et al., Arthritis Rheum., 41:694-700, 1998.
McGonagle et al., Curr. Opin. Rheumatol., 11:244-250, 1999.
McIver et. al., Pain, 120(1-2):161-9, 2005.
Mease et al., Lancet, 356:385-390, 2000.
Merrill and Benvenist, Trends Neurosci., 19:331-338, 1996.
Morris et. al., J. Mol. Med., 80(2):96-104, 2002.
Morse and Choi, Am. J. Respir. Crit. Care Med., 172(6):660-670, 2005.
Morse and Choi, Am. J. Respir. Crit. Care Med., 27(1):8-16, 2002.
Muthusamy et al., Free Radic. Biol. Med., 52(2):366-76, 2012.
Nachbauer et al., PLoS One, 8(7):e69229, 2013.
Nath et al., Neurology, 66(1):149-150, 2006.
Neal et al., BMJ., 314(7083):779-782, 1997.
Nichols, Drug News Perspect., 17(2):99-104, 2004.
Nishino et al., Cancer Res., 48:5210-5215, 1988.
Ohnishi et al., Int. Immunol., 6:817-830, 1994.
Osburn and Kensler, Mutat. Res., 659:31-39, 2008.
Pall, Med Hypoth., 69:821-825, 2007.
Partsch et al., Br. J. Rheumatol., 24:518-523, 1997.
Paupe et al., PLoS One, 4(1):e4253, 2009.
Peake and Whiting, Clin. Biochem. Rev., 27:173-184, 2006.
Pergola et. al., N Engl J Med, 365:327-336, 2011.
Pica et al., Antimicrob Agents Chemother., 44(1):200-4, 2000.
Pimentel et al., Am. J. Gastroenterol., 95(12):3503-3506, 2000.
Place et al., Clin. Cancer Res., 9:2798-2806, 2003.
Rajakariar et. al., Proc. Nat. Acad. Sci. USA, 104(52):20979-84, 2007.
Rawlins et al., Am. J. Clin. Pathol., 123:439-445, 2005.
Reagan-Shaw et al., FASEB J., 22(3):659-661, 2008.
Reimund et al., Eur. J. Clin. Invest., 28(2):145-150, 1998.
Repka, et al., Hot-melt extrusion technology. In: Enclopedia of Pharmaceutical Technology, 2^{nd} ed, New York, NY: Marcel Dekker, 2002: 203-206.
Rogler and Andus, World J. Surg., 22(4):382-389, 1998.
Rojas-Rivera et al., Int. J Nephrol., 2012:321714, 2012.
Rooney et al., Rheumatol. Int., 10:217-219, 1990.
Ross et. al., Am. J. Clin. Pathol., 120(Suppl):S53-71, 2003.
Ross et. al., Expert Rev. Mol. Diagn., 3(5):573-585, 2003.
Rostom et al., Ann. Intern. Med., 146, 376-389, 2007.
Ruster et. al., Scand. J. Rheumatol., 34(6):460-3, 2005.
Sacerdoti et. al., Curr Neurovasc Res., 2(2):103-111, 2005.
Saiki et al., Scand. J. Gastroenterol., 33(6):616-622, 1998.
Salomonsson et al., Scand. J. Immunol., 55(4):336-342, 2002.
Salvemini et. al., J. Clin. Invest., 93(5):1940-1947, 1994.
Sarchielli et. al., Cephalalgia, 26(9):1071-1079, 2006.
Satoh et. al., Proc. Natl. Acad. Sci. USA, 103(3):768-773, 2006.
Schlosstein et al., NE J. Medicine, 288:704-706, 1973.
Schneider et al., Annual Review of Pharmacology and Toxicology, 47:731-759, 2007.
Schulz et. al., Antioxid. Redox. Sig., 10:115, 2008.
Shan et al., Antioxid. Redox Signal., 19(13):1481-93, 2013.
Simonian and Coyle, Annu. Rev. Pharmacol. Toxicol., 36:83-106, 1996.
Sinha et al., Cancer Res., 67:4507-4513, 2007.
SPORANOX® (itraconazole) capsules [package insert]. Titusville, NJ. Janssen Pharmaceuticals, Inc. March 2017.
Stack et al., Lancet, 349(9051):521-524, 1997.
Stewart et al., Neurology, 48:626-632, 1997.
Suh et al., Cancer Res., 63:1371-1376, 2003.
Suh et al., Cancer Res., 58:717-723, 1998.
Suh et al., Cancer Res., 59(2):336-341, 1999.
Szabo et. al., Nature Rev. Drug Disc., 6:662-680, 2007.
Takahashi et. al., Cancer Res., 57:1233-1237, 1997.
Tamir and Tannebaum, Biochim. Biophys. Acta, 1288:F31-F36, 1996.
Targan et al., N. Engl. J. Med., 337(15):1029-1035, 1997.
Touzani et al., J. Neuroimmunol., 100(1-2):203-215, 1999.
Tumlin et al., Am. J. Cardiol., 98(6A):14K-20K, 2006.
Vachiéry and Davenport, European Respiratory Review, 18:260-271, 2009.
van den Berg, Semin. Arthritis Rheum., 30(5S-2):7-16, 2001.
van Dullemen et al., Gastroenterol., 109(1):129-135, 1995.
van Hogezand and Verspaget, Drugs, 56(3):299-305, 1998.
Vasan et al., Journal of the American College of Cardiology, 33:1948-1955, 1999.
Vazquez et al., J. Virol., 79(7):4479-91, 2005.
Wardle, Nephrol. Dial. Transplant., 16(9):1764-8, 2001.
Wermuth and Stahl, In: Pharmaceutical Salts: Properties, Selection and Use—A Handbook, Verlag Helvetica Chimica Acta, 2002.
Weyand and Goronzy, Ann. NY Acad. Sci., 987:140-149, 2003.
Williams et al., Clin. Neurosci., 2(3-4):229-245, 1994.
Wordsworth, In: Genes and Arthritis, Brit. Medical Bulletin, 51:249-266, 1995.
Wright, Clin. Orthop. Related Res., 143:8-14, 1979.
Wu et al., Am. J. Physiol. Renal Physiol., 300:F1180-1192, 2011.
Yoh et al., Kidney Int., 60(4):1343-1353, 2001.
Yu et al., Nat. Rev. Immunol., 7:41-51, 2007.
Zhou et al., Am. J. Pathol., 166(1):27-37, 2005.
Zhou et al., Cancer Sci., 98:882-889, 2007.
Zingarelli et al., J. Immunol., 171(12):6827-6837, 2003.
Zingarelli et al., Crit Care Med., 31, S105-S111, 2003.

1. A method of treating a patient having Friedreich's ataxia with a compound of the formula: 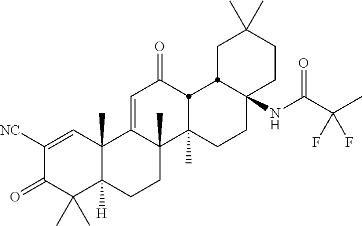
or a pharmaceutically acceptable salt or prodrug thereof;
the method comprising administering a therapeutically effective amount of the compound to the patient, wherein the patient is not currently taking a CYP3A4 modulator.

2. The method of claim 2,
comprising (i) determining or having determined whether a patient is currently being administered a CYP3A4 modulator; and (ii) selecting or having selected the patient for treatment with the compound if the patient is not currently being administered a CYP3A4 modulator.

3. (canceled)

4. The method of claim 1,
wherein the patient has discontinued concomitant use of a CYP3A4 modulator.

5-7. (canceled)

8. The method of claim 1, wherein the administration of a CYP3A4 modulator is avoided during administration of the compound.

9. The method of claim 1, wherein administration of the CYP3A4 modulator is discontinued prior to starting administration of the compound.

10-14. (canceled)

15. The method of claim 9, wherein
the CYP3A4 modulator is discontinued at least 1 week prior to starting administration of the compound.

16. The method of claim 1,
wherein the CYP3A4 modulator is a strong inhibitor of CYP3A4.

17. The method of claim 1,
wherein the CYP3A4 modulator is a moderate inhibitor of CYP3A4.

18. The method of claim 1,
wherein the CYP3A4 modulator is a moderate activator of CYP3A4.

19. The method of claim 1,
wherein the CYP3A4 modulator is a strong activator of CYP3A4.

20. The method of claim 1,
wherein the CYP3A4 modulator is itraconazole, clarithromycin, indinavir, nefazodone, saquinavir, suboxone, telithromycin, erythromycin, diltiazem, ketoconazole, ritonavir, goldenseal, aprepitant, erythromycin, fluconazole, grapefruit, verapamil, diltiazem, a barbiturate, carbamazepine, efavirenz, modafinil, nevirapine, oxcarbazepine, pioglitazone, rifavutin, troglitazone, phenobarbital, phenytoin, rifampicin, St. John's Wort, or a glucocorticoid.

21-49. (canceled)

50. The method of claim 1,
wherein the patient does not have an elevated BNP level.

51. The method of claim 50, wherein the patient has a BNP level less than or equal to 200 pg/mL.

52-152. (canceled)

153. The method of claim 1, wherein at least a portion of the compound is present as a polymorphic form having an X-ray powder diffraction pattern (CuKα) comprising a halo peak at about 14° 2θ.

154. The method of claim 153, wherein the X-ray powder diffraction pattern (CuKα) further comprises a shoulder peak at about 8° 2θ.

155. The method of claim 153, wherein the X-ray powder diffraction pattern (CuKα) is substantially as shown in FIG. 5.

156. The method of claim 153, further having a T_{g} from about 150° C. to about 155° C.

157. The method of claim 156, further having a T_{g} of about 153° C.

158. The method of claim 156, further having a T_{g} of about 150° C.

159. The method of claim 153, further having a differential scanning calorimetry (DSC) curve comprising an endotherm centered from about 150° C. to about 155° C.

160. The method of claim 159, wherein the endotherm is centered at about 153° C.

161. The method of claim 159, wherein the endotherm is centered at about 150° C.

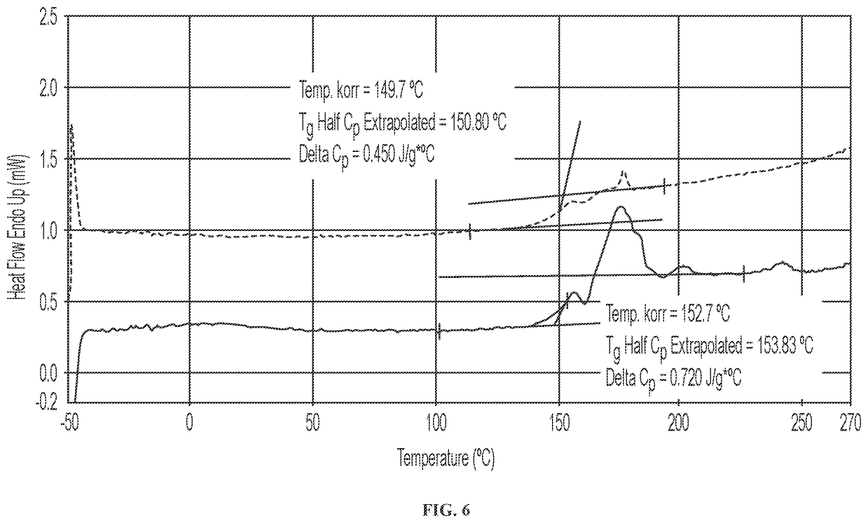

162. The method of claim 153, having a differential scanning calorimetry (DSC) curve substantially as shown in FIG. 6.

163. The method of claim 1, wherein at least a portion of the compound is present as a polymorphic form having a solvate having an X-ray powder diffraction pattern (CuKα) comprising significant peaks at about 5.6, 7.0, 10.6, 12.7, and 14.6° 2θ.

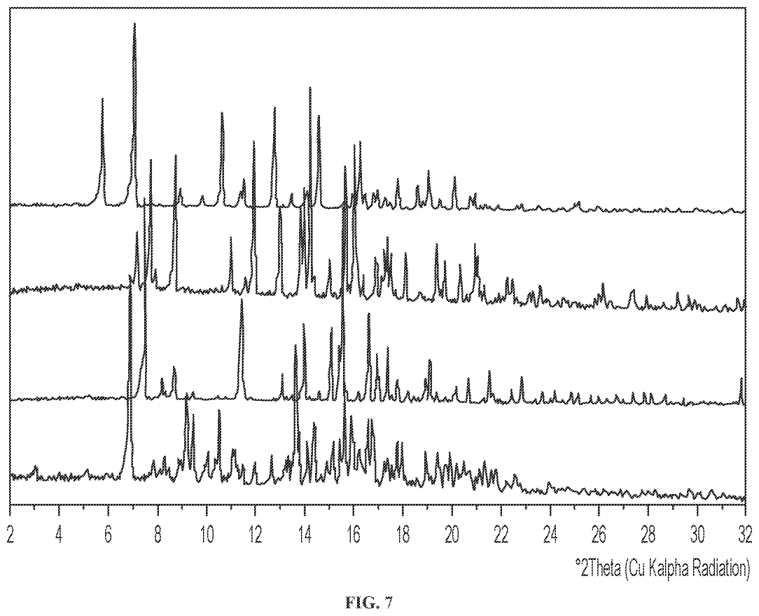

164. The method of claim 163, wherein the X-ray powder diffraction pattern (CuKα) is substantially as shown in FIG. 7, top pattern.

165. The method of claim 1, wherein at least a portion of the compound is present as a polymorphic form having a solvate having an X-ray powder diffraction pattern (CuKα) comprising significant peaks at about 7.0, 7.8, 8.6, 11.9, 13.9 (double peak), 14.2, and 16.0° 2θ.

166. The method of claim 165, wherein the X-ray powder diffraction pattern (CuKα) is substantially as shown in FIG. 7, second pattern from top.

167. The method of claim 1, wherein at least a portion of the compound is present as a polymorphic form having an acetonitrile hemisolvate having an X-ray powder diffraction pattern (CuKα) comprising significant peaks at about 7.5, 11.4, 15.6, and 16.6° 2θ.

168. The method of claim 167, wherein the X-ray powder diffraction pattern (CuKα) is substantially as shown in FIG. 7, second pattern from bottom.

169. The method of claim 167, further having a T_{g} of about 196° C.

170. The method of claim 167, further having a differential scanning calorimetry (DSC) curve comprising an endotherm centered at about 196° C.

171. The method of claim 167, having a differential scanning calorimetry (DSC) curve substantially as shown in FIG. 8.

172. The method of claim 1, wherein at least a portion of the compound is present as a polymorphic form having a solvate having an X-ray powder diffraction pattern (CuKα) comprising significant peaks at about 6.8, 9.3, 9.5, 10.5, 13.6, and 15.6° 2θ.

173. The method of claim 172, wherein the X-ray powder diffraction pattern (CuKα) is substantially as shown in FIG. 7, bottom pattern.

174. The method of claim 1, wherein at least a portion of the compound is present as a crystalline polymorphic form having an X-ray powder diffraction pattern (CuKα) comprising peaks at about 10.601, 11.638, 12.121, 13.021, 13,435, 15.418, 15.760, 17.830, 18.753, and 19.671° 2θ.

175. The method of claim 174, wherein the X-ray powder diffraction pattern (CuKα) is substantially as shown in FIG. 9.

176. The method of claim 174, wherein the melting point is about 181.98° C.

177. The method of claim 174, having a differential scanning calorimetry (DSC) curve substantially as shown in FIG. 10.

178. The method of claim 1, wherein at least a portion of the compound is present as a crystalline polymorphic form having an X-ray powder diffraction pattern (CuKα) comprising peaks at about 7.552, 10.339, 11.159, 12.107, 14.729, 15.329, 15.857, 16.824, 17.994, 18.344, 19.444, 19.764, 20.801, and 22.414° 2θ.

179. The method of claim 178, wherein the X-ray powder diffraction pattern (CuKα) is substantially as shown in FIG. 11.

180. The method of claim 178, wherein the melting point is about 250.100° C.

181. The method of claim 178, having a differential scanning calorimetry (DSC) curve substantially as shown in FIG. 12.

182. The method of claim 1, wherein the compound is administered in a single dose per day.

183-184. (canceled)

185. The method of claim 1, wherein the therapeutically effective amount is a daily dose of from about 25 mg to about 500 mg.

186. (canceled)

187. The method of claim 185, wherein the daily dose is about 150 mg.

188-191. (canceled)

192. The method of claim 1, wherein the therapeutically effective amount is a daily dose of 3-100 mg of compound per kg of body weight.

193-198. (canceled)

199. The method claim 1, wherein the compound is administered orally.

200-203. (canceled)
